SEQUENCE LISTING (25 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 70 aa, synthetic construct
  REGION 1..70: note = synthetic construct
GPRTLCGAELVDALQFVCGDRGFYFNKPTGAGSSSRRAPQTGIVDECCFRSCDLRRLEMYCAPLKPAKSA

SEQ ID NO: 3 - protein, 83 aa, synthetic construct
  REGION 1..83: note = synthetic construct
MFPAMPLSSLFVNGPETLCGAELVDALQFVCGDRGFYFNKPTGYGSSSRRAPQTGIVDECCFRSCDLRRLEMYCAPLKPAKSA

SEQ ID NO: 4 - protein, 70 aa, synthetic construct
  REGION 1..70: note = synthetic construct
GPRTLCGAELVDALQFVCGDRGFYFNKPTGYGSSSRRAPQTGIVDECCFRSCDLRRLEMYCAPLKPAKSA

SEQ ID NO: 5 - protein, 67 aa, synthetic construct
  REGION 1..67: note = synthetic construct
TLCGAELVDALQFVCGDRGFYFNKPTGYGSSSRRAPQTGIVDECCFRSCDLRRLEMYCAPLKPAKSA

SEQ ID NO: 6 - protein, 83 aa, synthetic construct
  REGION 1..83: note = synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 7 - protein, 69 aa, synthetic construct
  REGION 1..69: note = synthetic construct
GPETLCGAELVDALQFVCGDRGFYFNKPTGYGSSSRAPQTGIVDECCFRSCDLRRLEMYCAPLKPAKSA

SEQ ID NO: 8 - protein, 67 aa, synthetic construct
  REGION 1..67: note = synthetic construct
GPETLCGAELVDALQFVCGDRGFYFNKPTGYGSSSRRAPQTGIVDECCFRSCDLRRLEMYCAPLKPA

SEQ ID NO: 9 - protein, 319 aa, Homo sapiens
AQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 10 - protein, 319 aa, synthetic construct
  REGION 1..319: note = synthetic construct
AQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLAGEDD

SEQ ID NO: 11 - protein, 319 aa, synthetic construct
  REGION 1..319: note = synthetic construct
AQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 12 - protein, 585 aa, Homo sapiens [sequence omitted]

SEQ ID NO: 13 - protein, 585 aa, synthetic construct
  REGION 1..585: note = synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGL

SEQ ID NO: 14 - protein, 585 aa, synthetic construct
  REGION 1..585: note = synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAGTFTFHADICTLSEKERQIKKQTALVELVKHKPKATKEQLKAAMDDFAAFVEKCCKADDKETCFAEEGKKLVAASQAALGL

SEQ ID NO: 15 - protein, 585 aa, synthetic construct; identical to SEQ ID NO: 13
  REGION 1..585: note = synthetic construct

SEQ ID NO: 16 - protein, 20 aa, synthetic construct
  REGION 1..20: note = synthetic construct
GGGGSGGGGSGGGGSGGGGS

SEQ ID NO: 17 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 18 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 19 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 20 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 21 - protein, 20 aa, synthetic construct
  REGION 1..20: note = synthetic construct
ASPAAPAPASPAAPAPSAPA

SEQ ID NO: 22 - protein, 107 aa, synthetic construct
  REGION 1..107: note = synthetic construct
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIK

SEQ ID NO: 23 - protein, 7 aa, synthetic construct
  REGION 1..7: note = synthetic construct
GSGGGSG

SEQ ID NO: 24 - protein, 988 aa, synthetic construct
  REGION 1..988: note = synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLGSGGGSGAQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLAGEDD

SEQ ID NO: 25 - protein, 988 aa, synthetic construct
  REGION 1..988: note = Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLGSGGGSGAQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLSGEDD

(19) United States
(12) Patent Application Publication
(10) Pub. No.: US 2026/0226132 A1
(43) Pub. Date: Aug. 6, 2026

(54) METHODS OF TREATMENT USING IGF-1 CHIMERIC PROTEINS

(71) Applicant: Silver Creek Pharmaceuticals, Inc., San Francisco, CA (US)

(72) Inventors: Terry O'Reilly, Gold Canyon, AZ (US); Yan Zhang, Sausalito, CA (US); Kristopher M. Kuchenbecker, Phoenix, AZ (US); Samuel J. Pfaff, Wyncote, PA (US); Lakhmir Chawla, San Diego, CA (US)

(73) Assignee: Silver Creek Pharmaceuticals, Inc., San Francisco, CA (US)

(21) Appl. No.: 19/152,246

(22) PCT Filed: Feb. 2, 2024
(86) PCT No.: PCT/US2024/014251

§ 371 Date: Jul. 30, 2025

Related U.S. Application Data
(60) Provisional application No. 63/482,904, filed on Feb. 2, 2023.
(60) Provisional application No. 63/588,481, filed on Oct. 6, 2023.

(51) Int. Cl. C07K 14/65; A61K 31/7004; A61K 38/00; A61P 9/10; A61P 25/28; A61P 39/00; C07K 14/435

(52) U.S. Cl. CPC C07K 14/65; A61K 31/7004; A61P 9/10; A61P 25/28; A61P 39/00; C07K 14/435; A61K 38/00; C07K 2319/035; C07K 2319/31; C07K 2319/33

(57) ABSTRACT

Provided herein are methods of treating a subject in need thereof with an IGF-1 chimeric protein while supporting euglycemia. The IGF-1 chimeric protein comprises a targeting domain comprising non-internalizing human annexin 5 and the composition comprising the IGF-1 chimeric protein is administered in combination with a dextrose solution to support euglycemia.

### RELATED APPLICATIONS

This application claims the benefit of and priority to U.S. Provisional Application Ser. No. 63/482,904, filed Feb. 2, 2023, and U.S. Application Ser. No. 63/588,481, filed Oct. 6, 2023, each of which is incorporated herein by reference in its entirety.

### REFERENCE TO SEQUENCE LISTING

This specification includes a sequence listing submitted herewith, which includes the file entitled 132463-011202.xml having the following size: 37,142 bytes which was created Feb. 1, 2024, the contents of which are incorporated by reference herein.

### TECHNICAL FIELD

Aspects of the present disclosure relates generally to pharmaceutical compositions comprising IGF-1 chimeric proteins, and methods for using such IGF-1 chimeric proteins in a subject in need thereof.

### BACKGROUND

The insulin-like growth factors (IGFs) constitute a family of proteins having insulin-like and growth stimulating properties.

### SUMMARY

Aspects of the disclosure relate to methods of treating a subject in need thereof with an IGF-1 chimeric protein, the method comprising administering a solution comprising an effective amount of dextrose prior to administering an IGF-1 chimeric protein, wherein the administering is over a period of about 48 hours, and administering a pharmaceutical composition comprising an effective amount of the IGF-1 chimeric protein, wherein the effective amount of dextrose supports euglycemia. In some embodiments, the solution comprising the effective amount of dextrose is administered by infusion to the subject in need thereof. In some embodiments, the method comprises administering the solution comprising the effective amount of dextrose at a starting infusion rate of about 0.1 mL/kg/h. In some embodiments, the method further comprises adjusting the infusion rate at an incremental rate of +0.05 mL/kg/hr to +2 mL/kg/hr.

In some embodiments, the solution comprising the effective amount of dextrose comprises from about 5% to about 10% dextrose (w/v) in water. In some embodiments, the solution comprising the effective amount of dextrose comprises from about 5% to about 10% dextrose (w/v) in saline solution.

In some embodiments, the method comprises administering the solution comprising the effective amount of dextrose from about 1 min to about 30 min before the administration of the IGF-1 chimeric protein.

In some embodiments, the IGF-1 chimeric protein comprises a targeting domain comprising human annexin 5 (AnxV) or variant thereof, and an activator domain comprising insulin-like growth factor (IGF-1) or variant thereof. In some embodiments, the IGF-1 chimeric protein further comprising a peptide, wherein the peptide extends the half-life of the chimeric protein. In some embodiments, the targeting domain is a non-internalizing variant of annexin 5. In some embodiments, the IGF-1 chimeric protein is substantially not internalized by cells. In some embodiments, the IGF-1 chimeric protein comprises a non-internalizing variant of annexin 5, wherein the non-internalizing variant of annexin 5 comprises one or more mutations, wherein the one or more mutations comprises a substitution at a position corresponding to C316 and optionally at one or more positions corresponding to R63, K70, K101, E138, D139, N160, and combinations thereof. In some embodiments, the activator domain of chimeric protein is a variant of human insulin-like growth factor IGF-1 comprising one or more mutations, wherein the one or more mutations consist of a substitution at one or more positions corresponding to E3, Y24, Y31, Y60, and combinations thereof. In some embodiments, the IGF-1 chimeric protein further comprises a half-life modulator comprising a variant of human serum albumin (HSA) comprising one or more mutations, wherein the one or more mutations consist of a substitution at one or more positions corresponding to C58 and N527, and combinations thereof. In some embodiments, the IGF-1 chimeric protein comprises or consists of IGF1(E3R/Y31A)_lk7_HSA26-609(C58S/N527Q)_lk7_AnxV2-320(R63A/K70A/K101A/E138A/D139G/N160A/C316A). In some embodiments, the IGF-1 chimeric protein comprises or consists of an amino acid sequence as set forth in SEQ ID NO: 24.

In some embodiments, the method comprises administering descending effective amounts of the IGF-1 chimeric protein over a period of 2 days to 14 days or more.

In some embodiments, the administering of the effective amount of the IGF-1 chimeric protein inhibits apoptosis.

In some embodiments, the pharmaceutical composition further comprises at least one physiologically acceptable carrier.

In some embodiments, the subject in need thereof is a human.

In some embodiments, the subject in need thereof has acute CNS injury.

In some embodiments, the subject in need thereof has acute cardiovascular injury.

In some embodiments, the subject in need thereof has acute radiation sickness.

In some embodiments, the subject in need thereof has chemical inhalation injury.

In some embodiments, the subject in need thereof has dermatologic injuries.

In some embodiments, the subject in need thereof has traumatic injuries.

In some embodiments, the subject in need thereof has chronic neurodegenerative diseases.

In some embodiments, the subject in need thereof is undergoing a procedure with risk of iatrogenic injury.

In some embodiments, the subject in need thereof is undergoing an organ transplantation procedure.

In some embodiments, the subject in need thereof is undergoing cosmetic dermatologic treatments.

Some aspects of the disclosure relate to an IGF-1 chimeric protein for use in a method of treating a subject in need thereof, the method comprising administering a solution comprising an effective amount of dextrose prior to administration of the IGF-1 chimeric protein, wherein the administering is over a period of about 48 hours; and administering a pharmaceutical composition comprising an effective amount of the IGF-1 chimeric protein, wherein the effective amount of dextrose supports euglycemia.

Some aspects of the disclosure relate to dextrose for use in a method of treating a subject in need thereof with an IGF-1 chimeric protein, the method comprising administering a solution comprising an effective amount of said dextrose prior to administration of the IGF-1 chimeric protein, wherein the administering is over a period of about 48 hours; and administering a pharmaceutical composition comprising an effective amount of the IGF-1 chimeric protein, wherein the effective amount of dextrose supports euglycemia.

Some aspects of the disclosure relate to combination of an IGF-1 chimeric protein and dextrose for use in a method of treating a subject in need thereof, the method comprising administering a solution comprising an effective amount of dextrose prior to administration of the IGF-1 chimeric protein, wherein the administering is over a period of about 48 hours; and administering a pharmaceutical composition comprising an effective amount of the IGF-1 chimeric protein, wherein the effective amount of dextrose supports euglycemia.

Some aspects of the disclosure relate to the use of an IGF-1 chimeric protein in the manufacture of a medicament for treating a subject in need thereof, wherein a solution comprising an effective amount of dextrose is administered prior to administration of a pharmaceutical composition comprising an effective amount of the IGF-1 chimeric protein IGF-1 chimeric protein, wherein administration of the solution is over a period of about 48, wherein the effective amount of dextrose supports euglycemia.

Some aspects of the disclosure relate to the use of dextrose with an IGF-1 chimeric protein in the manufacture of a medicament for treating a subject in need thereof, wherein a solution comprising an effective amount of the dextrose is administered prior to administration of a pharmaceutical composition comprising an effective amount of the IGF-1 chimeric protein IGF-1 chimeric protein, wherein administration of the solution comprising the effective amount of the dextrose is over a period of about 48, wherein the effective amount of dextrose supports euglycemia.

Some aspects of the disclosure relate to the use of a combination of IGF-1 chimeric protein and dextrose in the manufacture of a medicament for treating a subject in need thereof, wherein a solution comprising an effective amount of the dextrose is administered prior to administration of a pharmaceutical composition comprising an effective amount of the IGF-1 chimeric protein IGF-1 chimeric protein, wherein administration of the solution comprising the effective amount of the dextrose is over a period of about 48, wherein the effective amount of dextrose supports euglycemia.

### DESCRIPTION OF THE SEQUENCE LISTING

SEQ ID NO: 1 is the amino acid sequence of wild-type human IGF-1 (mature form).

SEQ ID NO: 2 is the amino acid sequence of a variant of wild-type human IGF-1 variant comprising E3R and Y31A substitutions.

SEQ ID NO: 3 is the amino acid sequence a variant of human IGF-1 (IGF-1 LONG). SEQ ID NO: 4 is the amino acid sequence a variant of human IGF-1 (IGF1 E3R).

SEQ ID NO: 5 is the amino acid sequence of a variant of human IGF-1 (IGF-1Des 1-3). SEQ ID NO: 6 is the amino acid sequence of a variant of human IGF-1 (IGF-1 LR3).

SEQ ID NO: 7 is the amino acid sequence a variant of human IGF-1 (IGF1 R37X).

SEQ ID NO: 8 is the amino acid sequence of human a variant of human IGF-1 with deletion of residues 68-70 (IGF1 3X).

SEQ ID NO: 9 is the amino acid sequence of wild type human annexin A5 (AnxV).

SEQ ID NO: 10 is the amino acid sequence of a variant of wild-type human annexin 5 comprising the amino acids 2-320 of wild type annexin 5 and the R63A, K70A, K101A, E138A, D139G, N160A and C316A substitutions.

SEQ ID NO: 11 is the amino acid sequence of non-internalizing variant of human annexin A5 (ni-AnxV).

SEQ ID NO: 12 is the amino acid sequence of wild type Human Serum Albumin (HSA).

SEQ ID NO: 13 is the amino acid sequence of Human Serum Albumin variant mHSA (C34S, N503Q substitutions).

SEQ ID NO: 14 is the amino acid sequence of Human Serum Albumin variant mHSA7 (C34S, N503Q, E505G and V547A substitutions).

SEQ ID NO: 15 is the amino acid sequence of a variant human serum albumin comprising the amino acids 26-609 of wild type human serum albumin and the C58S and N527Q substitutions.

SEQ ID NO: 16 is the amino acid sequence of a peptide linker.

SEQ ID NO: 17 is the amino acid sequence of human transferrin (Tf).

SEQ ID NO: 18 is the amino acid sequence of Human Alpha Fetoprotein (AFP).

SEQ ID NO: 19 is the amino acid sequence of Human Vitamin D Binding Protein (VDBP).

SEQ ID NO: 20 is the amino acid sequence of Human Transthyretin (TTR).

SEQ ID NO: 21 is the amino acid sequence of a motif PASylation.

SEQ ID NO: 22 is the amino acid sequence of the albumin-binding domain human antibody (aldudAB).

SEQ ID NO: 23 is the amino acid sequence of a peptide linker lk7.

SEQ ID NO: 24 is the amino acid sequence of chimeric protein scp776.

SEQ ID NO: 25 is the amino acid sequence of chimeric protein IGF1(E3R/Y31A)_lk7_HSA26-609(C58S/N527Q)_lk7_AnxV2-320(R63A/K70A/K101A/E138A/D139G/N160A/C316S).

### BRIEF DESCRIPTION OF THE DRAWINGS

FIG. 1 is a non-quantitative schematic representation of an organ tissue response to an acute injury either treated with scp776 or untreated.

FIG. 2A is a scheme showing interaction of scp776 with healthy cells or apoptotic cells.

FIG. 2B is a graph showing pro-survival signaling per scp776 concentration in healthy and injured cells.

FIG. 3 is a graph showing mean serum scp776 concentrations plotted for three cohorts in Phase 1A study SCP-CL-0001. Inset is a graph showing the same data on a semi-log scale.

FIG. 4 is a graph showing the calculated areas above 70 mg/dL blood glucose (euglycemic area) from blood glucose versus time profiles of subjects in Phase 1A study SCP-CL-0001(-dextrose) and Phase 1B study SCP-CL-0002 (+dextrose).

FIG. 5A and FIG. 5B are graphs showing the preclinical efficacy of scp776 in an NHP model of AIS. FIG. 5A: Study PR0362-1 compared three groups: Placebo (n=12), Low Dose (24 mg/kg scp776 total; n=10), and High Dose (32 mg/kg scp776 total; n=10). Dose dependence was noted for all measured parameters. For simplicity, only the high dose is shown. Blood-brain barrier permeability (Gadolinium Contrast, p=0.0325), lesion size (T2 MRI, p=0.0164), and neurological dysfunction (neurological deficit score, p=0.0323) are depicted for the high dose and placebo groups as group means+SEM. Multiple comparison of means (ANOVA with least significant difference) was performed with all three groups to determine significant effects. Values for monkeys that perished prior to a scheduled assessment were imputed to the maximum observed value. FIG. 5B: Survival curves for the high dose scp776 (n=10) and placebo (n=12) groups from Study PR0362-1 are plotted.

FIG. 6 shows patient flow.

FIG. 7 shows blood glucose trajectory (Example 1). Blood glucose trajectories are plotted as circles and colored according to individual mice (n=8). The averaged response is plotted as the blue diamonds and connecting line.

FIG. 8 is a graph showing total dextrose infusion for healthy human subjects. Cohort 1: Filled Circle, Cohort 2: Filled Square, Cohort 3: Filled Triangle, Cohort 4: Open Circle, Cohort 5: Open Square, Cohort 6: Open Triangle

FIG. 9 is a diagram showing the pharmacology and systemic effects of the IGF-1 component of scp776 can be addressed by dextrose supplementation.

FIG. 10 is a table showing the Phase 1a SAD study and Phase 1b MAD study.

FIG. 11A and FIG. 11B are graphs showing that supplemental dextrose decreases frequency of hypoglycemic events. FIG. 11C is a table showing that supplemental dextrose decreases frequency of hypoglycemic events.

### DETAILED DESCRIPTION

It is to be understood that the terminology used herein is for the purpose of describing particular embodiments of the disclosure only and is not intended to be limiting.

Unless defined otherwise, all technical and scientific terms used herein have the same meaning as commonly understood by one having ordinary skill in the art to which the disclosure pertains.

All publications, patents and patent applications cited herein, whether supra or infra, are hereby incorporated by reference in their entirety.

### Definitions

As used in this specification and the appended claims, the singular forms “a, “an” and “the” include plural referents unless the content clearly dictates otherwise.

The term “peptide,” “polypeptide” and “protein” are used interchangeably to denote a sequence polymer of at least two amino acids covalently linked by an amide bond (also referred herein as peptide bond).

As used herein the term “target molecule” refers to any molecule that is associated with a tissue (e.g. “at risk”, diseased or damaged tissue). A “target cell” is meant to be a cell to which a protein or targeting domain thereof can specifically bind.

“Binding” or “specific binding” are used interchangeably herein and indicates that a protein (or the targeting polypeptide domain thereof or the activator domain thereof) exhibits substantial affinity for a specific molecule (e.g., targeting domain exhibits substantial affinity for a target molecule, or an activator domain exhibits substantial affinity for a molecule associated with the surface of a cell such as a growth factor receptor) or a cell or tissue bearing the molecule and is said to occur when the protein (or the targeting polypeptide domain thereof or the activator domain thereof) has a substantial affinity for a specific molecule and is selective in that it does not exhibit significant cross-reactivity with other molecules.

“Identity,” as known in the art, is a relationship between two or more polypeptide or protein sequences, as determined by comparing the sequences. In the art, “identity” also refers to the degree of sequence relatedness between polypeptides or proteins, as determined by the match between strings of such sequences. “Identity” can be readily calculated by any bioinformational methods known in the art.

The term “parent polypeptide” refers to a wild-type polypeptide and the amino acid sequence or nucleotide sequence of the wild-type polypeptide is part of a publicly accessible protein database (e.g., EMBL Nucleotide Sequence Database, NCBI Entrez, ExPasy, Protein Data Bank and the like).

The term “mutant polypeptide” or “polypeptide variant” refers to a form of a polypeptide, wherein its amino acid sequence differs from the amino acid sequence of its corresponding wild-type (parent) form, naturally existing form or any other parent form. A mutant polypeptide can contain one or more mutations, e.g., substitution, insertion, deletion, addition etc., which result in the mutant polypeptide. Generally, variants are overall closely similar, and, in many regions, identical to the reference polypeptide. As used herein, “variant” refers to a polypeptide, differing in sequence from a native protein but retaining at least one functional and/or therapeutic property thereof as described elsewhere herein or otherwise known in the art.

The term “corresponding to a parent polypeptide” is used to describe a polypeptide of the disclosure, wherein the amino acid sequence of the polypeptide differs from the amino acid sequence of the corresponding parent polypeptide only by the presence of at least one amino acid variation. Typically, the amino acid sequences of the variant polypeptide and the parent polypeptide exhibit a high percentage of identity. In one example, “corresponding to a parent polypeptide” means that the amino acid sequence of the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the amino acid sequence of the parent polypeptide. In another example, the nucleic acid sequence that encodes the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the nucleic acid sequence encoding the parent polypeptide.

The term “substantial identity” or “substantial similarity,” as used herein, when referring to a nucleic acid or fragment thereof, indicates that when optimally aligned with appropriate nucleotide insertions or deletions with another nucleic acid (or its complementary strand), there is nucleotide sequence identity in at least about 95% to 99% of the sequence. The term “substantial identity” or “substantial similarity,” as used herein, when referring to a protein or fragment thereof, indicates that when optimally aligned there is an amino acid sequence identity in at least about 95% to 99% of the sequence.

The term “damaged cell” or “damaged tissue,” as used herein, means and includes biological cell or tissue; for example, but not limited to, neurons, glia or nervous tissue damaged or injured by, but not limited to, trauma or chemical insult, ischemic tissue, cell or tissue damaged by any means which results in interruption of normal blood flow to the tissue.

The term “therapeutically effective amount,” as used herein, means the amount of the protein or agent that will elicit the biological or medical response of a tissue, system, animal or human that is being sought by the researcher, veterinarian, medical doctor or other clinician.

The term “pharmaceutically acceptable,” or “physiologically acceptable” as used herein, means the carrier, diluent or excipient must be compatible with the other ingredients of the formulation and not deleterious to the recipient thereof.

The term “targeting moiety”, “targeting domain,” “targeting polypeptide” or “targeting module” are used herein interchangeably and refer to molecules that selectively localize the chimeric protein in a particular tissue or region of the body. The localization can be mediated by specific recognition of molecular determinants, molecular size of the targeting domain, ionic interactions, hydrophobic interactions and the like. As used herein, the terms “therapeutic moiety,” “activator domain,” “activator polypeptide”, “signaling arm” and “effector module” are used herein interchangeably and refers to any agents useful for therapy and that are non-toxic, do not have a cytotoxic effect or are not detrimental to the cells. Such agents can include, but not limited to, growth factors.

A “patient” or a “subject” is a mammal, preferably a human. The term “treating” (or “treat” or “treatment”) means slowing, reducing, or reversing the progression or severity of a symptom, disorder, condition, or disease.

The term “therapeutically effective amount” refers to the amount or dose of chimeric proteins described herein which, upon single or multiple dose administration to a patient, provides the desired treatment.

As used herein, the term “physiologically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the bi-specific fusion protein is administered. Physiologically acceptable carriers can be sterile liquids, such as water and oils, including those of petroleum, animal, vegetable or synthetic origin (e.g., peanut oil, soybean oil, mineral oil, or sesame oil). Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water and ethanol. The pharmaceutical composition, if desired, can also contain minor amounts of wetting or emulsifying agents, or pH buffering agents.

Like insulin, endogenous IGF-1 has known blood glucose lowering effects, which is a demonstrated dose limiting toxicity of exogenously administered IGF-1. Endogenous IGF-1, which shares approximately 50% of the amino acid sequencing of insulin, has broad systemic metabolic and homeostasis effects in the body. Its direct effects on glucose metabolism are mediated through pancreatic insulin secretion and increased glucose uptake by muscle tissue due to increased insulin sensitivity. When administered to severely insulin resistant diabetic patients, IGF-1 improved glucose levels, but also resulted in significant systemic adverse events at doses required for these therapeutic effects.

### IGF-1 Chimeric Proteins

Chimeric proteins provided herein are capable of specific binding to two or more different specific molecules. In some embodiments, the chimeric protein comprises a targeting domain having a binding specificity to a first specific target molecule, an activator domain having a binding specificity to a second target molecule, and a half-life modulator.

In some aspects, the activator domain has a binding specificity to a tyrosine kinase receptor at the cell surface. In some embodiments, the binding of the activator to the tyrosine kinase receptor activates intracellular signaling pathways associated with cell survival. In some aspects, the activator domain has a binding specificity to a receptor that modulates/promotes tissue regeneration.

In some embodiments, the targeting domain serves to target the chimeric protein to a target cell or tissue while the activator domain serves to activate the intracellular signaling pathway associated with cell survival.

In some embodiments, the half-life modulator extends the half-life of the chimeric protein.

In some embodiments, the chimeric proteins are fusion proteins having a targeting polypeptide connected or linked to a half-life modulator and to an activator polypeptide. In some embodiments, the engineered proteins are chimeric proteins having a targeting polypeptide connected or linked to a half-life modulator and a growth factor or mutated growth factor.

In some embodiments, the mutated growth factor (e.g. IGF-1 variant) is engineered to reduce potency while retaining the ability to activate the cognate growth factor receptor. In some embodiments, wild type growth factors can be used as activator domains.

The targeting domain is generally used to target the chimeric proteins to a target cell. In some embodiments the target cell is undergoing apoptosis. The binding of the targeting domain to its target molecule does not induce a significant biological effect in the target cell. The activator domain binds to a receptor on a cell surface. The binding of the activator domain to its receptor is intended to modulate a specific biological effect, such as, activate the intracellular signaling pathway associated with cell survival. In some embodiments, binding of the activator domain to its receptor is intended the positively regulate survival of the targeted cells or tissue. In particular, the activator domain of chimeric protein can promote survival signaling.

In some embodiments, the in vivo activity of the chimeric protein can be assessed by detecting signaling changes in molecules that are regulated by the activator domain, including but not limited to cell surface receptor phosphorylation status or downstream mediators such as phospho-AKT or phospho-ERK (as detected by flow cytometry, immunofluorescence, ELISA, phospho-labeling, Western analysis of treated tissues, or any other methodology known in the art.) In some embodiments, a chimeric protein functions in vivo if it induces a significant (e.g., at least 10%, at least 20%, at least 30%, at least 40%, at least 50% or more) change in the level, functional activity, or phosphorylation of the regulated molecule detected by the assay.

### Activator Domain

The activator domain can be any polypeptide that detectably modulates the activity of a cellular network. In some embodiments, the activator domain is capable of activating signal transduction pathways by binding to a receptor at the surface a cell. In some embodiments, certain activator domains are growth factor polypeptides, or any agonist of the receptor. It will be apparent that such modulation may be an increase in the activity of the cellular network such as induction of proliferation of cells, induction of cell growth, promotion of cell survival and/or inhibition of apoptosis.

During acute injury, cells have 3 fates: survival, apoptosis, or necrosis. Some injured cells can naturally avoid cell death via a mechanism called apoptosis escape. Enhancement of apoptosis escape after an acute injury leads to improved cell survival and preserved organ function. Growth factors have the potential to promote cell and tissue survival by enhancing apoptosis escape.

An activator domain for a particular application may be selected based on the desired therapeutic outcome. For example, to increase survival and neuroprotection, activator domains that comprise IGF-1 (or variant or fragment thereof) can be used.

In some embodiments, the activator domain comprises a change in the amino acid sequence, the three-dimensional structure of the protein, and/or the activity of the protein, relative to the wild-type form of the protein.

In some embodiments, the activator domain comprises or consists of a growth factor having amino acid sequence modification relative to the wild-type growth factor (e.g. IGF-1) to decrease its binding to its natural receptor (e.g. IGF-1 receptor), to decrease its binding to binding proteins (e.g. IGF binding proteins) and/or decrease its activation of its natural receptor (e.g. IGF-1 receptor). In some embodiments, the activator domain is a growth factor having amino acid sequence modification that reduce (e.g., for about 1-5%, 5-10%, 10%-20%, about 20%-40%, about 50%, about 40%-60%, about 60%-80%, about 80%-90%, 90-95%) the binding to its natural receptor (e.g. IGF-1 receptor).

A growth factor polypeptide detectably modulates activation of a growth factor receptor. In some embodiments, the activator domain of the bi-specific protein is a growth factor, variant or fragment thereof that retains at least about 0.01% of wild-type biological activity. In some embodiments, the activator domain of the bi-specific protein is a growth factor, variant or fragment thereof that retain at least about 0.1%, at least about 1%, at least about 10%, of wild-type biological activity. In some embodiments, the activator domain of the bi-specific protein is a growth factor, variant or fragment thereof that retains between about 0.01% to about 0.1% of wild-type biological activity. In some embodiments, the activator domain of the bi-specific protein is a growth factor, variant or fragment thereof that retains between about 0.01% to about 1% of wild-type biological activity. In some embodiments, the activator domain of the bi-specific protein is a growth factor, variant or fragment thereof that retains between about 0.01% to about 10% of wild-type biological activity. In some embodiments, the activator domain of the bi-specific protein is a growth factor, variant or fragment thereof that retains between about 0.1% to about 1% of wild-type biological activity. In some embodiments, the activator domain of the bi-specific protein is a growth factor, variant or fragment thereof that retains between about 0.1% to about 10% of wild-type biological activity. In some embodiments, the activator domain of the bi-specific protein is a growth factor, variant or fragment thereof that retains between about 01% to about 10% of wild-type biological activity. Biological activity in some embodiments can be determined by measuring activation of the corresponding growth factor receptor in appropriate cells. In some embodiments, activation may be assessed, for example, by measuring phosphorylation of receptor kinase or downstream effector proteins, such as, but not limited to, AKT, S6, ERK, JNK, mTOR, etc.

### Insulin-Like Growth Factors (IGFs) and Derivatives Thereof

The insulin-like growth factors (IGFs) constitute a family of proteins having insulin-like and growth stimulating properties. The IGFs Human IGF-1 is a 70 amino acids basic peptide having the protein shown in SEQ ID NO: 1, respectively. IGF-1 and extracellular tyrosine kinase receptor (e.g. IGF-1 receptor) are important for cellular processes such as cell proliferation and survival. Binding of IGF-1 or variant thereof to the IGF-1 receptor stimulates kinase activity, leading to phosphorylation of multiple substrate, thereby initiating signaling cascades. The chimeric proteins disclosed herein can maintain the ability to signal through the extracellular receptor, for example IGF-1 receptor. The activator domain IGF-1 stimulates cell proliferation and survival through activation of the AKT pathway. Upon binding of IGF-I to the IGF-1 receptor, a tyrosine kinase, phosphorylates tyrosine residues on two major substrates, IRS-1 and Shc, which subsequently signal through the Ras/Raf and PI 3-kinase/AKT pathways.

The interaction of IGF-1 (and IGF-2) with the IGF-1 receptor is regulated by IGF binding Proteins (IGFBPs). All six IGFBPs (particularly IGFBP5) have been shown to inhibit IGF action, but in some instances a stimulatory effect has been observed. At least 99% of the IGF in the circulation is normally bound to IGFBPs.

In some embodiments, the activator domain is a variant of the human IGF-1 or fragment thereof. In some embodiments, the variant of IGF-1 or fragment thereof is capable of maintaining selectivity to the IGF-1 receptor.

In some embodiments, the IGF-1 variant is modified to reduce binding to IGF-1 binding proteins (IGFBPs) relative to wild-type IGF-1 while maintaining its ability to activate the AKT pathway. In some embodiments, the IGF-1 variant can activate the IGF-1 receptor with a decreased potency for non-target cells, as assessed by pAKT EC50. EC50 is defined as the concentration needed to achieve the half maximal level of pAKT signaling.

In some embodiments, the IGF-1 variant comprises a substitution at one or more of the tyrosine residues. In some embodiments, the IGF-1 variant comprises one or more substitutions at position Y24, Y31 and Y60. In an exemplary embodiment, the IGF-1 variant can comprise a single tyrosine substitution at position Y31, or Y24, or Y60. In an exemplary embodiment, the IGF-1 variant can comprise a single tyrosine substitution at position Y24 and Y31, Y24 and Y60, Y31 and Y60, or Y24 and Y60. In another exemplary embodiment, the IGF-1 variant can comprise one or more of the following substitutions, Y24L, Y31A, and Y60L relative to wild type IGF-1. For example, the IGF-1 variant can comprise the Y24L substitution and the Y31A substitution or the IGF-1 variant can comprise the Y24L substitution, the Y31A substitution and the Y60L. In some embodiments, one or more tyrosine residues (Y24, Y31, Y60 or combinations thereof) can be substituted for a short aliphatic amino acid. In some embodiments, one or more tyrosine residues (Y24, Y31, Y60 or combinations thereof) can be substituted for a polar amino acid. In some embodiments, one or more tyrosine residues (Y24, Y31, Y60 or combinations thereof) can be substituted for leucine, alanine, isoleucine, serine, threonine or any other amino acid.

In some embodiments, the IGF-1 variant comprises a substitution replacing Arg for a Glu, Lys, Met, Val, Ala, Leu, Ile, Gly, Ser, or Thr at the 3 position of the polypeptide. In some embodiments, the IGF-1 variant comprises a substitution replacing Arg for a Glu at the 3 position of the polypeptide (E3R).

In some embodiments, the IGF-1 variant comprises a substitution at the position 3 and 31. For example, the IGF-1 variant comprises E3R and Y31A substitutions. In some embodiments, the activator domain has an amino acid sequence having SEQ ID NO: 2.

In some embodiments, the activator domain is a derivative of the human IGF-1 comprising one or more of the following modifications: a N-terminal 13-residue extension (IGF-1 LONG), a deletion of amino acids 1-3 (Des-1-3), a substitution replacing Arg for a Glu at the 3 position of the polypeptide (E3R), no Arginine at position 37 (R37X), a deletion of amino acids 68-70 (3X), an N-terminal 13-residue extension and a substitution replacing Arg for a Glu at the 3 position of the wild-type polypeptide (LR3), substitutions of one or more of tyrosine residues (Y24, Y31, Y60 or combinations thereof (e.g. Y24L, Y31A, Y60L substitutions or combinations thereof).

In some embodiments, the activator domain is variant of the human IGF-1 comprising a mutation (e.g. substitution, deletion) at one or more residues 24 to 37.

In some embodiments, the activator domain is a derivative of the human IGF-1 and comprises an N-terminal 13-residue extension (also referred as IGF-1 LONG, SEQ ID NO: 3), a mutation E3R (SEQ ID NO: 4) or a combination thereof (LONG E3R, also referred as LR3, SEQ ID NO: 6). In some embodiments, the IGF-1 variant comprises the E3R substitution, an N-terminal 13-residue extension, deletion of amino acids 1-3 ((Des1-3), SEQ ID NO: 5) or a combination thereof to decrease the binding of the activator domain to the IGF binding proteins which are present in the serum and other body fluid.

In some embodiments, the activator domain is a derivative of the human IGF-1 and comprises one or more of the following modifications: an N-terminal 13-residue extension (SEQ ID NO: 3), a deletion of amino acids 1-3 (SEQ ID NO: 5), a substitution replacing Arg for a Glu at the 3 position of the polypeptide (SEQ ID NO: 4), no Arginine at position 37 (R37X, SEQ ID NO: 7), a deletion of amino acids 68-70 (3X, SEQ ID NO: 8), or an N-terminal 13-residue extension and a substitution replacing Arg for a Glu at the 3 position of the wild-type polypeptide (SEQ ID NO: 6).

It is believed that the bi-specific proteins that contain the variant of IGF-1 described herein (e.g. E3R, IGF-1 LONG, IGF-1 LONG E3R (referred to as IGF-1 (LR3)) or IGF1 Des1-3), have decreased affinity for IGF binding proteins relative to wild-type IGF-1. In some embodiments, the IGF-1 variants of the bi-specific proteins described herein can activate the signaling pathway while having a substantially decreased interaction with the IGF-1 binding proteins relative to wild-type IGF-1.

In some embodiments, the IGF-1 variant can be modified by glycosylation of one or more glycosylation site present in the IGF-1 variant.

In some embodiments, the chimeric proteins that contain the IGF-1 variants described herein have a potency for non-target cells that is less than wild-type IGF-1 for non-target cells.

Certain activator domains that bind to growth factor receptors are provided herein in SEQ ID NOs: 1-8.

Additional peptide sequence modifications can be included, such as variations, deletions, substitutions or derivatizations of the amino acid sequence of the sequences disclosed herein, so long as the peptide has substantially the same activity or function as the unmodified peptides. Notably, a modified peptide will retain activity or function associated with the unmodified peptide, the modified peptide will generally have an amino acid sequence “substantially homologous” with the amino acid sequence of the unmodified sequence.

In some embodiments, the IGF-1 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NOs: 1-8. In some embodiments, the IGF-1 variant can have an amino acid sequence having from about 85% to about 90%, from about 90% to about 95%, from about 95% to about 98%, from about 98% identity to about 99% identity to the amino acid sequence provided in SEQ ID NOs: 1-8. In some embodiments, the IGF-1 variant can comprise 10, 20, 30, 40, 50, 60 or more consecutive amino acids of any one of amino acids in SEQ ID NOs: 1-8. In some embodiments, the IGF-1 variant can have an amino acid sequence recited in any one of SEQ ID NOs: 1-8. In some embodiments, the IGF-1 variant can have an amino acid sequence recited in any one of SEQ ID NOs: 2-8 In some embodiments, the IGF-1 variant can have an amino acid sequence recited in SEQ ID NO: 2.

In some embodiments, the bi-specific protein comprises an activator domain having a growth factor variant such as an IGF-1 variant that is selected to give the bi-specific protein at least an order of magnitude lower EC50 in damaged tissue than in healthy tissue. For example, the bi-specific protein domain comprises a growth factor variant such as an IGF-1 variant and has an EC50 in damaged tissue that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower than the EC50 in healthy tissue.

In some embodiments, the bi-specific proteins that contain the IGF-1 variants have a half maximal effective concentration (EC50) that is lower in damaged tissue than in healthy tissue. In some embodiments, the bi-specific proteins that contain the IGF-1 variants have a half maximal effective concentration (EC50) that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower in damaged tissue than in healthy tissue.

In some embodiments, the chimeric proteins provided herein having such variant growth factors have a higher specificity to the damaged tissue targeted.

### Target Molecules

In some aspects, target molecules are exposed or enriched on the exterior of a target cell. In some embodiments, the target molecule is associated with a damaged cell, early apoptotic or apoptotic cell, the target molecule being intracellular in a viable or undamaged cell and being exposed to the extracellular space in a damaged cell. Such molecules include, for example, molecules that are exposed in cells that undergo necrosis (such as DNA) or apoptosis (e.g., phosphatidylserine), myosin (including the tissue type-specific subtypes thereof), ICAM-1 or P-selectin. Yet in other embodiments, the target molecule is a molecule that is present or enriched at the surface of a diseased or dysfunctional cell or tissue as compared to the level detected in a healthy or functional cell or tissue. In some embodiments, the target cell is not a tumor or cancerous cell.

Cells are bounded by a plasma membrane (or cell membrane) comprising a lipid bilayer. The cell membrane may be considered to have a surface facing the cytosol (cytosolic side or interior of the cell) and a surface facing the exterior of the cell, or the extracellular space. Trans-bilayer movement of anionic phospholipids from the inner to the outer leaflet of the plasma membrane occurs during apoptosis. The anionic phospholipid-binding protein, such as Annexin A5, synaptotagmin I or lactadherin can be used to detect the presence of phosphatidylserine on the outer leaflet of the cell membrane. Phosphatidylserine is a phospholipid, that is usually restricted to the cytosolic side of the membrane in viable or undamaged cells, and that becomes exposed on the outer cell surface or to the extracellular space in damaged cells or apoptosis.

In some embodiments, the target molecule is an “ischemia-associated molecule”. An “ischemia-associated molecule” is any molecule that is detected at a level that is significantly higher (e.g., at least 1.5 higher, at least 2-fold higher, at least 3-fold higher, at least 4-fold higher, at least 5-fold higher) following ischemia (which results in hypoxia) or hypoxia than in a cell of the same tissue that has not undergone an ischemic event (i.e., the molecule is specific to or enriched in the post-ischemic tissue). Ischemia occurs when there is insufficient blood flow to provide adequate oxygenation, which results in tissue hypoxia (reduced oxygen) or anoxia (absence of oxygen) as the most severe form of hypoxia, and ultimately tissue necrosis, and apoptosis.

### Targeting Domain

In some embodiments, the targeting domain has a specific binding affinity to a target molecule associated with a tissue (for example, an ischemia-associated molecule). In some embodiments, the targeting domain has a specific binding affinity for a target molecule presented on the surface of early apoptotic cells. The targeting domain may be any polypeptide sequence that serves this function. In some embodiments, binding of the targeting domain to the target molecule does not have or does not modulate a biological activity. As used herein, “biological activity” refers to a defined, known activity performed by exposure of a molecule to a domain of the protein.

In some embodiments, the targeting domain can be a non-antibody polypeptide, fragment thereof or variant thereof having a binding affinity to the target molecule. Yet in other embodiments, the targeting polypeptide domain comprises one or more antibody variable regions (e.g. scFv).

### Annexin A5 and Variants Thereof

In some embodiments, the targeting domain comprises annexin, a variant thereof or a fragment thereof. The term “annexin” refers to any protein capable of binding to phospholipids, especially phosphatidylserine (PS), and member of the annexin family. In some embodiments, the annexin is Annexin A5 but other annexins can equally be used. In some embodiments, the targeting domain is human Annexin A5, a functional fragment thereof, or a variant thereof. A variant of Annexin A5 comprises at least one amino acid in at least one position in which this amino acid is not found in the parent wild type Annexin A5 polypeptide (SEQ ID NO: 9). The annexin variants according may comprise one or more amino acid substitutions, deletions, additions, or combinations thereof wherein the amino acid substitutions, deletions, or additions do not substantially affect the ability of the Annexin A5 variant of the chimeric protein to bind to at least one phospholipid, such as PS. In some embodiments, the Annexin A5 variant can have an amino acid sequence having at least about 85%, at least about 90%, at least about 95%, at least about 98% identity or at least about 99% identity to the amino acid sequence provided in SEQ ID NO: 9. In some embodiments, the Annexin A5 variant can comprise 50, 80, 100, 110, 200, 300, or more consecutive amino acid having at least about 85%, at least about 90%, at least about 95%, at least about 98% identity or at least about 99% identity to the amino acids in SEQ ID NO: 9.

In some embodiments, the variant of Annexin A5 is modified to substitute cysteine at position 315 (corresponding to C316) with serine or alanine to reduce dimer formation. For example, the cysteine can be substituted to an alanine or a serine. As used herein, the term “corresponding to” is used to designate the position/identity of an amino acid residue in a polypeptide (e.g., Annexin A5). Those of ordinary skill will appreciate that, for purposes of simplicity, a canonical numbering system (based on wild-type Annexin A5) is utilized herein, so that an amino acid “corresponding to” a residue at position 316, for example, need not actually be the 316th amino acid in a particular amino acid chain but rather corresponds to the residue found at position 316 in a for example Annexin A5 before the post-translational removal of the N-terminal methionine; those of ordinary skill in the art readily appreciate how to identify corresponding amino acids. In particular, it is noted that the amino acid sequence of wild-type Annexin A5 (SEQ ID NO: 9) do not start with a Methionine as the Methionine residue is cleaved during processing.

In some embodiments, the variant of Annexin A5 has an amino acid sequences that has been mutated to reduce internalization of Annexin A5 or the chimeric protein comprising the variant of Annexin A5 into a cell while maintaining binding affinity to phosphatidylserine (PS). In some embodiments, the variant of Annexin A5 or the chimeric protein comprising the variant of Annexin A5 has a binding affinity to phosphatidylserine, and is not internalized into a cell or is internalized at a slower rate than wild-type annexin A5. In some embodiments, the targeting domain is a non-internalizing variant of Annexin A5, (also referred as ni-Annexin A5 or ni-AnxV, SEQ ID NO: 11). In some embodiments, the variant of Annexin A5 has an amino acid set forth in SEQ ID NO: 10. In some embodiments, the non-internalizing mutant of Annexin A5 has an amino acid sequence having at least about 85%, at least about 90%, at least about 95%, at least about 98% identity or at least about 99% identity to the amino acid sequence provided in SEQ ID NO: 11. In some embodiments, the non-internalizing mutant of Annexin A5 can have an amino acid sequence having from about 85% to about 90%, from about 90% to about 95%, from about 95% to about 98%, from about 98% to about 99% identity to the amino acid sequence provided in SEQ ID NO: 11. In some embodiments, the Annexin A5 variant can comprise 50, 80, 100, 110, 200, 300, or more consecutive amino acid of any one of amino acids in SEQ ID NO: 11. Any variation of Annexin A5 that results in substantially no internalization is envisioned.

It should be appreciated that the non-internalizing variant of annexin A5 can confer an extended half-life to the chimeric protein as compared to a chimeric protein that contains wild-type A5. In some embodiments, the variants of annexin A5 that results in substantially no internalization, or chimeric proteins containing variants of annexin A5 that results in substantially no internalization, can have an extended half-life of 1.1 to 1.2, 1.1 to 1.3, 1.1. to 1.4, 1.1 to 1.5, 1.1 to 1.6, 1.1 to 1.7, 1.1 to 1.8, 1.1 to 1.9, 1.1 to 2 or greater as compared to wild-type annexin A5, or chimeric proteins containing wild-type annexin A5.

The terms “non-internalizing” and “substantially no internalization,” as used herein, refer to a lack of internalization of a substantial amount of the chimeric protein disclosed herein. For example, the phrase “substantially no internalization” will be understood as less than 50% of the chimeric protein being internalized by a cell to which the chimeric protein is bound, or less than 25% of the chimeric protein being internalized by a cell to which the chimeric protein is bound, or less than 10% of the chimeric protein being internalized by a cell to which the chimeric protein is bound, or less than 5% of the chimeric protein being internalized by a cell to which the chimeric protein is bound, or less than 3% of the chimeric protein being internalized by a cell to which the chimeric protein is bound, or less than 1% of the chimeric protein being internalized by a cell to which the bi-specific protein is bound.

In some embodiments, the non-internalizing mutant of Annexin A5 can have an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% identity or at least about 99% identity to human Annexin A5. In some embodiments, the non internalizing variant of Annexin A5 comprises a substitution at position 315 (corresponding to C316) wherein the cysteine residue is substituted with serine (Ser), alanine (Ala), leucine (Leu), phenylalanine (Phe), methionine (Met) or tryptophan (Trp). In some embodiments, the non-internalizing mutant of Annexin A5 can have an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% identity or at least about 99% identity to human Annexin A5. In some embodiments, the non-internalizing variant of Annexin A5 comprises a substitution at position 315 (corresponding to C316) wherein the cysteine residue is substituted with serine (Ser) or alanine (Ala). In some embodiments, the non-internalizing mutant of Annexin A5 can have an amino acid sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% identity or at least about 99% identity to human Annexin A5 modified to substitute cysteine at position 315 (corresponding to C316) with serine or alanine.

In some embodiments, Annexin A5 or Annexin A5 variants (for example variant having a substitution at position C316) are modified to comprise one or more substitutions at the following positions: R62, K69, K100, E137, D138, N159, L313 (corresponding to R63, K70, K101, E138, D139, N160, L314 relative to wild type human Annexin A5).

In some embodiments, the Annexin A5 variant comprises:
R62A, R62E, R62D, R62M, R62L, R62I, R62Y (corresponding to R63A, R63E, R63D, R63M, R63L, R63I, R63Y relative to wild type human Annexin A5);
K69A, K69E, K69D, K69M, K69L, K69I, K69Y (corresponding to K70A, K70E, K70D, K70M, K70L, K70I, K70Y relative to wild type human Annexin A5);
E137A, E137K, E137R, E137M, E137L, E137I, E137Y (E138A, E138K, E138R, E138M, E138L, E138I, E138Y relative to wild type human Annexin A5);
D138G, D138K, D138R, D138M, D138L, D138I, D138Y (corresponding to D139G, D139K, D139R, D139M, D139L, D139I, D139Y relative to wild type human Annexin A5)
N159A, N160M, N160L, N160I, N160V, N160Y (corresponding to N160A, N160M, N160L, N160I, N160V, N160Y relative to wild type human Annexin A5)
L313E L313D, L313K, L313R, L313H, L313Q, L313N, L313Y (corresponding to L314E L314D, L314K, L314R, L314H, L314Q, L314N, L314Y relative to wild type human Annexin A5);
or any combinations of the foregoing.

In some embodiments, Annexin A5 or Annexin A5 variants comprise one or more substitutions at position D143 and/or E227. In some embodiments, Annexin A5 variant comprises:
D142G, D142A, D142K, or D142R (corresponding to D143G, D143A, D143K, or D143R) substitution, and/or
E226G, E226A, E226K, or E226R (corresponding to E227G, E227A, E227K, E227R) substitution.

In some embodiments, Annexin A5 or Annexin A5 variants (for example having a substitution at C316, D143 and/or E227) are modified to comprise one or more of the following substitutions R62A, K69A, K100A, E137A, D138G, N159A, L313E (corresponding to R63A, K70A, K101A, E138A, D139G, N160A, L314E). For example, Annexin A5 having SEQ ID NO: 9 can be modified to have C315A or C315S substitution (corresponding to C316A or C316S relative to wild type Annexin A5) and one or more of the following substitutions R62A, K69A, K100A, E137A, D138G, N159A, L313E (corresponding to R63A, K70A, K101A, E138A, D139G, N160A, L314E relative to wild type Annexin A5).

In some embodiments, human Annexin A5 (SEQ ID NO: 9) are modified to comprise one or more of the following substitutions R62A, K69A, K100A, E137A, D138G, N159A, D143N, E227A, C315S or C315A (corresponding to R63A, K70A, K101A, E138A, D139G, D144N, N160A, E228A, C316S or C316A relative to wild type Annexin A5).

In some embodiments, the targeting domain is Annexin A5 which has been engineered to have R63A, K70A, K101A, E138A, D139G, N160A and C316A or C316S substitutions relative to wild type Annexin A5. For example, the targeting domain can have the amino acid sequence of SEQ ID NO: 10.

In some embodiments, the Annexin A5 variant comprises one, two, three, four, five, six, seven, eight, nine, ten, eleven, twelve, thirteen, fourteen, fifteen, sixteen, seventeen, eighteen nineteen, twenty or more substitutions in different regions, in order to further decrease the internalization of the annexin in a cell. For example, the Annexin A5 variants may comprise R62A and K69A, R62A and K100A, R62A and E137A, R62A and D138G, R62A and N159A, R62A and K69A and K100A, R62A and K69A and E137A, R62A, K69A and K100A, R62A, K69A, K100A, and E137A etc. . . .

The annexin variants according may further comprise one or more amino acid substitutions, deletions, or additions, wherein the amino acid substitutions, deletions, or additions do not substantially affect the ability of the Annexin A5 variant of the chimeric protein to bind to at least one phospholipid, such as PS.

Native polypeptide can be used as targeting domains. It will be apparent, however, that portions of such native sequences and polypeptides having altered sequences may also be used, provided that such polypeptides retain the ability to bind the target molecule with an appropriate binding affinity (Kd) as described in more details below.

### Antibody Targeting Domain:

In some embodiments, an anti-phosphatidylserine antibody can be used as a targeting domain. As used herein, term “antibody” includes but is not limited to: (i) a Fab fragment, a monovalent fragment consisting of the VL, VH, CL and CH1 domains; (ii) F(ab)2 and F(ab′)2 fragments, a bivalent fragment comprising two Fab fragments linked by a disulfide bridge at the hinge region; (iii) a Fd fragment consisting of the VH and CH1 domains; (iv) a scFv fragment consisting of the VL and VH domains of a single arm of an antibody, (v) a dAb fragment which consists of a VH domain; and (vi) an isolated complementarity determining region (CDR). Such antibodies may be produced from intact antibodies using methods known in the art, or may be produced recombinantly, using standard recombinant DNA and protein expression technologies.

### Binding of Targeting Domain

In some embodiments, the chimeric protein binds to the target molecule with a Ka of less than 10⁻⁶ M, preferably less than 10⁻⁷ M, 10⁻⁸ M, 10⁻⁹ M or 10⁻¹⁰ M.

### Half-Life Modulator

One skilled in the art would appreciate that proteins used in therapeutic applications may not exhibit optimal serum half-lives due to their relatively low molecular weight. In some therapeutic applications, it may therefore be desirable to extend the half-life of the proteins. In some embodiments, to achieve accumulation of the chimeric protein to the diseased injured or damaged area of an organ, the chimeric protein is conjugated operatively associated or fused with a half-life modulator. Preferably, the half-life modulator is non-immunogenic polypeptide.

For example, short half-life is the most limiting attribute of wild-type growth factors as therapeutics. Intravenous administered IGF-1 has a serum half-life in humans of less than 1 hour. The extended half-life of chimeric proteins disclosed herein compared to IGF-1, for example, allows for 1) equivalent efficacy with less frequent dosing; 2) equivalent exposure at a lower dose; 3) lower Cmax at an equivalent exposure level, reducing the risk of Cmax-related toxicity.

In some embodiments, the half-life modulators can increase the in vivo half-life of the chimeric proteins. For example, the half-life of the chimeric proteins comprising the half-life modulator is about 1 hour, 2 hour, 3 hours, 4 hours, 5 hours, 6 hours or greater. For example, the half-life of the chimeric proteins can be about 8 hours or more when tested in cynomolgus monkey. In some embodiments, the half-life of the chimeric proteins comprising the half-life modulator is about 24 hours, or greater. In some embodiments, the half-life of the chimeric proteins comprising the half-life modulator is about a week or greater.

In some embodiments, the half-life modulator is non-immunogenic in humans.

In some embodiments, the half-life modulator is a polypeptide that interacts with cellular machinery that promote evasion of lysosomal degradation pathways (e.g.—FcRn receptor-mediated recycling).

In some embodiments, the half-life modulator is designed to extend the half-life of the chimeric protein through binding to serum components such as Human Serum Albumin (HSA). HSA is the most abundant protein in the blood and has a demonstrated safety in humans.

In some embodiments, the half-life modulator is an HSA variant. In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human serum albumin amino acid sequence (wtHSA, SEQ ID NO: 12). In some embodiments, the half-life modulator comprises at least 200 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 300 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 400 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 500 consecutive amino acids that are at least 70%, 80%, 85%, 90%, 91%, 92%, 93%, 94%, 95%, 96%, 97%, 98%, or 99% identical to wild type human serum albumin amino acid sequence.

In some embodiments, the HSA variant can have one of more of the following substitutions:
cysteine C58 can be substituted, for example, with serine (C58S), alanine (C58A), asparagine (C58N), leucine (C58L), or glutamine (C58Q),
lysine K420 can be substituted for example, with glutamic acid (K420E), aspartic acid (K420D), a leucine (K420L) or methionine (K410M),
asparagine N527 can be substituted for example, with glutamine (N527Q), aspartic acid (N527D), histidine (N527H), or tyrosine (N527Y),
glutamic acid E505 can be substituted for example, with glycine (E505G), alanine (E505A), leucine (E505L), lysine (E505K), valine (E505V), isoleucine (E5051), methionine (E505M), or glutamine (E505Q),
valine V547 can be substituted for example, with alanine (V547A), glycine (V547G), leucine (V547L), lysine (V547K), isoleucine (V5471), methionine (V547M),
asparagine N503 can be substituted for example, with a Glutamine (N527Q), aspartic acid (N503D), histidine (N503H), or tyrosine (N503Y), or glutamine (V547Q).

In some embodiments, the HSA variant can have amino acids 26-609 and have one of more of the following substitutions:
cysteine C58 can be substituted for example, with serine (C58S), alanine (C58A), asparagine (C58N), leucine (C58L), or glutamine (C58Q),
lysine K420 can be substituted for example, with glutamic acid (K420E), aspartic acid (K420D), a leucine (K420L) or methionine (K410M),
asparagine N527 can be substituted for example, with glutamine (N527Q), aspartic acid (N527D), histidine (N527H), or tyrosine (N527Y),
glutamic acid E505 can be substituted for example, with a glycine G (E505G), alanine (E505A), leucine (E505L), lysine (E505K), valine (E505V), isoleucine (E5051), methionine (E505M), or glutamine (E505Q),
valine V547 can be substituted for example, with an alanine (V547A), glycine (V547G), leucine (V547L), lysine (V547K), isoleucine (V5471), methionine (V547M),
asparagine N503 and/or N527 can be substituted for example, with Glutamine (N503Q and/or N527Q), aspartic acid (N503D and/or N527D), histidine (N503H and/or N527H), or tyrosin (N503Y and/or N527Y).

In some embodiments, the HSA variant (referred herein as mHSA) has the following substitutions: C34S, N503Q (SEQ ID NO: 13). In some embodiments, the HSA variant (referred herein as mHSA7) has the following substitutions C34S, N503Q, E505G and V547A (SEQ ID NO: 14). In some embodiments, the HSA variant has amino acids 26-609 and the following substitutions C58S and N527Q (SEQ ID NO: 15).

In some embodiments, the asparagine at position 503 and/or 527 of HSA, which may be deamidated and decrease half-life, can be removed by the N503Q substitution and/or the N527Q. In some embodiments, the cysteine C34 of HSA may be substituted to serine or alanine (S or A) to remove the free cysteine and minimize alternate disulfide-bond formation. In some embodiments, the half-life modulator is a modified version of the domain III (mHSA_dIII) of a modified HSA with the N503Q substitution and an additional terminal glycine. Such a modified version retains the HSA property of binding to FcRn and increased serum half-life.

In some embodiments, the half-life modulator is an Fc domain of an antibody or a single chain constant fragment. In some embodiments, the half-life modulator comprises Fc regions of an immunoglobulin molecule (e.g. IgG). In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a human Fc amino acid sequence. The Fc domain of an antibody has a natural capability to bind FcRn, resulting in an extended half-life. In some embodiments, the Fc domain of an antibody is engineered not to bind Fc (gamma) R In an exemplary embodiment, the Fc domain is engineered to substitute N297 with Q (N297Q variant). In some embodiments, the half-life modulator is a monomeric variant form of Fc (scFc). For example, the subset of IgG heavy chain which naturally dimerizes to form Fc is hinge-CH2-CH3. In some embodiments, the Fc domain is engineered to form a single chain by linking the hinge-CH2-CH3 with a flexible linker such as GGGGSGGGGSGGGGSGGGGS (SEQ ID NO: 16) to create a hinge-CH2-CH3-linker-hinge-CH2-CH3 chain. In an exemplary embodiment, the single chain Fc (scFc) is engineered to substitute N297 with Q and C220 with S (N297Q, C220S).

In some embodiments, the half-life modulator is a single chain variable fragment (scFv) of an antibody targeted to albumin or other circulating protein. In some embodiments, the half-life modulator comprises an amino acid sequence that is at least 70%, 80%, 85%, 90%, 91%, 92%, 93%, 94%, 95%, 96%, 97%, 98%, or 99% identical to scFv amino acid sequence directed to a specific antigen, such as, but not limited to, albumin. In some embodiments, the half-life modulator comprises at least 50, at least 100, at least 150, at least 200, at least 250 consecutive amino acids that are at least 70%, 80%, 85%, 90%, 91%, 92%, 93%, 94%, 95%, 96%, 97%, 98%, or 99% identical to a scFv amino acid sequence directed to a specific antigen, such as, but not limited to, albumin.

In some embodiments, the half-life modulator is transferrin such as human transferrin (Tf, SEQ ID NO: 17). In some embodiments, the half-life modulator comprises an amino acid sequence that is at least 70%, 80%, 85%, 90%, 91%, 92%, 93%, 94%, 95%, 96%, 97%, 98%, or 99% identical to human transferrin amino acid sequence. In some embodiments, the half-life modulator comprises at least 100, at least 200, at least 300, at least 400, at least 500, at least 600, at least 650 consecutive amino acids that are at least 70%, 80%, 85%, 90%, 91%, 92%, 93%, 94%, 95%, 96%, 97%, 98%, or 99% identical to a human transferrin amino acid sequence.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human alpha-fetoprotein amino acid sequence (AFP, SEQ ID NO: 18). In some embodiments, the half-life modulator comprises at least 100 consecutive amino acids that are about 70%, 80%, 85%, 90%, 91%, 92%, 93%, 94%, 95%, 96%, 97%, 98%, or 99% identical to wild type human alpha-fetoprotein (AFP) amino acid sequence. In some embodiments, the N-linked glycosylation site of the AFP is removed by the N251Q substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical wild-type vitamin D-binding protein amino acid sequence (VDBP, SEQ ID NO: 19). In some embodiments, the half-life modulator comprises at least 100 consecutive amino acids that are about 70%, 80%, 85%, 90%, 91%, 92%, 93%, 94%, 95%, 96%, 97%, 98%, or 99% identical wild-type vitamin D-binding protein (VDBP) amino acid sequence. In some embodiments, the N-linked glycosylation site of the VDBP can be removed by the N288Q or N288T substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild-type human transthyretin amino acid sequence (TTR, SEQ ID NO: 20). In some embodiments, the half-life modulator comprises at least 100 consecutive amino acids that are about 70%, 80%, 85%, 90%, 91%, 92%, 93%, 94%, 95%, 96%, 97%, 98%, or 99% identical to wild type human transthyretin (TTR) amino acid sequence. In some embodiments, the transthyretin is modified to remove the N118 N-glycosylation site. In some embodiments, the half-life modulator is a monomeric form of TTR.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation (see WO/2008/155134). In some embodiments, the half-life modulator comprises at least 100 consecutive amino acids that are about 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation. Polypeptide stretches of proline, alanine, and/or serine form semi-structured three-dimensional domains with large hydrodynamic radius, thereby reducing clearance of fusion proteins. In some embodiments, the PASylation amino acid sequence is about 200, 300, 400, 500 or 600 amino acids long. For example, the PASylation is a 20 times repeat of the amino acid sequence ASPAAPAPASPAAPAPSAPA (SEQ ID NO: 21).

In some embodiments, the half-life modulator comprises the attachment of polyethylene glycol (PEG) chain or chains to the fusion proteins through chemical attachment either to the N- and/or C-terminus and/or to an amino acid side chain (e.g., PEG-maleimide attachment to cysteines). PEG chains form semi-structured three-dimensional domains with large hydrodynamic radius, thereby reducing clearance of fusion proteins.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to an albumin-binding domain human antibody (albudAb) amino acid sequence (SEQ ID NO: 22). In some embodiments, the half-life modulator comprises at least 100 consecutive amino acids that are about 70%, 80%, 85%, 90%, 91%, 92%, 93%, 94%, 95%, 96%, 97%, 98%, or 99% identical to an albumin-binding domain human antibody (albudAb) amino acid sequence. Albumin-binding domain antibodies can increase the fusion protein half-life by binding non-covalently to serum albumin (see WO2008/096158 which is incorporated herein by reference in its entirety). In some embodiments, the albumin-binding domain human antibody is engineered to remove the C-terminal arginine to remove the Lys-Arg Kex2 protease site.

Representative such half-life modulators include those recited in any one of SEQ ID NOs: 12-15, 17-22.

In some embodiments, the half-life modulators can be modified to substitute the cysteine residues to serine or alanine residues to reduce the ability to form disulfide bonds.

In some embodiments, the targeting domain and activator domain can be joined via a half-life modulator. Accordingly, the half-life modulator can have two termini, an N-terminus and a C-terminus. In some embodiments, the half-life modulator is joined at one terminus via a peptide bond to the targeting polypeptide domain and is joined at the other terminus via a peptide bond to the activator domain. In certain embodiments, the half-life modulator is joined at the N-terminus to the C-terminus of the targeting polypeptide domain and at the C-terminus to the N-terminus of the activator domain. In other embodiments, the half-life modulator is joined at the C-terminus to the targeting polypeptide domain and at the N-terminus to the activator domain. Yet, in other embodiments, the half-life modulator is joined at one of the termini of the bi-specific protein. For example, in some embodiments, the half-life modulator is joined at the C-terminus to the N-terminus of the activator domain. In other embodiments, the half-life modulator is joined at the N-terminus to the C-terminus of the targeting domain. In other embodiments, the half-life modulator can be joined at the N-terminus to the C-terminus of the activator domain. Yet in other embodiments, the half-life modulator can be joined at the N-terminus to the C-terminus of the targeting domain.

### Peptide Linkers

In some embodiments, the activator domain, half-life modulator, and targeting domain are linked by peptide linker (e.g., from 2 to 40, 2-50, 2-100 amino acid residues) such that upon target recognition and engagement by the targeting domain, the presentation of the activator domain is optimized for binding to and activation of extracellular receptors on the surface of cells that present the target at a given surface density (e.g. −5×10² molecules/1,000 Å2)

Targeted delivery of the activator domain for example IGF-1 for the activation of receptors on cells or tissues displaying a specific target requires appropriate presentation of both the activator domain and the targeting domain. In some embodiments, the flexibility of the linker is optimized for proper geometry of the engaged chimeric protein. Some of the principal determinants of the geometric constraints are the distances from the cell surface for the target and the receptor.

Additional optimization can be driven by the relative number of receptors and target molecules. At high ratios of Receptor: Target molecule, the engagement of both domains is reaction-limited. When the target molecule is more abundant than the receptor, the occupancy of both domains is diffusion-limited. Under the reaction-limit, optimal delivery of the activator domain is attained via short and rigid linkers. Under the diffusion limit, long and flexible linkers allow the activator domain to access a larger surface area. For cells with complex shapes (i.e.-bodies and neuronal processes) and receptor distributions, appropriate design of linker flexibility can enable precise targeting to sub-cellular regions.

In some embodiments, the peptide linker is present at the N-terminus, at the C-terminus or at both the N-terminus and the C-terminus of the half-life modulator at one or both ends. Suitable short connector polypeptides for use at the N-terminal end of the linker include, for example, dipeptides such as -Gly-Ser-(GS), -Gly-Ala-(GA) and -Ala-Ser-(AS). Suitable peptide linkers for use at the C-terminal end of the linker include, for example, dipeptides such as-Leu-Gln-(LQ) and -Thr-Gly-(TG). In some embodiments, the peptide linkers are longer than 2 amino acids. For example, the peptide linkers are 5, 6, 7, 8, 9, 10, 11, 12, 13, 14, 15, 16, 17, 18, 19 or 20 amino acids long or longer. In some embodiments, the peptide linkers are 20 or more 30 or more, 40 or more, 50 or more, 60 or more, 70 or more, 80 or more, 90 or more, 100 or more amino acids long. Preferably, such peptide linkers are flexible (for example glycine-rich) or structured (e.g., alpha-helix rich). In some embodiments, the linker comprises or consist of amino acids—Gly-Ser-Gly-Gly-Gly-Ser-Gly (SEQ ID NO: 23).

It will be apparent that elements in addition to those described above may optionally be included in the proteins provided herein. Such elements may be present for a variety of purposes, including to facilitate expression, preparation or purification of the bi-specific fusion protein, or to perform targeting functions.

### Representative Chimeric Protein

In some embodiments, a representative bi-specific fusion proteins comprise (from N-terminal to C-terminal):
(a) a targeting polypeptide domain comprising or consisting of a non-internalizing human annexin V variant (e.g., comprising or consisting of amino acids 2-320 of wt human Annexin 5 and a substitution at C316, R63, K40, K101, E138, D139, N160);

(b) a linker peptide
(e.g., -Gly-Ser-Gly-Gly-Gly-Ser-Gly);

(c) a half-life modulator (e.g., HSA variant comprising or consisting of amino acids 26-609 of wt human HSA and comprising substitution at C58 and N527);

(d) linker peptide
(e.g., -Gly-Ser-Gly-Gly-Gly-Ser-Gly);

(e) an activator domain comprising or consisting of an IGF-1 variant (e.g., comprising a substitution at E3 and Y31.

In some embodiments, the chimeric protein comprises or consists of IGF1(E3R/Y31A)_lk7_HSA26-609(C58S/N527Q)_lk7_AnxV2-320(R63A/K70A/K101A/E138A/D139G/N160A/C316A) (also referred herein as scp776). In some embodiments, the chimeric protein has and amino acid sequence as set forth in SEQ ID NO: 24.

In some embodiments, the chimeric protein comprises or consists of IGF1(E3R/Y31A) lk7 HSA26-609(C58S/N527Q)_lk7_AnxV2-320(R63A/K70A/K101A/E138A/D139G/N160A/C316S). In some embodiments, the chimeric protein has an amino acid sequence as set forth in SEQ ID NO: 25.

### Nucleic Acids

Provided herein are polynucleotides encoding the chimeric proteins that may be in the form of RNA or in the form of DNA, which DNA includes cDNA and synthetic DNA. The DNA may be double-stranded or single-stranded. The coding sequences that encode the variants of the present disclosure may vary as a result of the redundancy or degeneracy of the genetic code.

### Pharmaceutical Compositions-Methods of Treatment

Pharmaceutical compositions comprising a therapeutically effective amount of at least one chimeric protein as described herein, together with at least one physiologically acceptable carrier, are provided. Such compositions may be used for treating patients who are suffering from, or at risk for, tissue damage, in order to prevent tissue damage, or to repair or regenerate damaged tissue.

In some embodiments, the subject in need thereof has acute CNS injury, acute cardiovascular injury (e.g., STEMI, Cardiac Arrest), acute radiation sickness (e.g., acute radiation syndromes (ARS) of the gastrointestinal (GI-ARS) and hematopoietic (H-ARS) systems), chemical inhalation injury (e.g., sulfur mustards exposure), dermatologic injuries (e.g., wound healing), traumatic injuries (e.g., burn, crush, laceration, contusion, avulsion, concussion, fracture, amputation), or chronic neurodegenerative diseases (e.g., synucleinopathies, amyloidoses). In some embodiments, the subject in need thereof is undergoing a procedure with risk of iatrogenic injury (e.g., transcatheter aortic valve replacement, percutaneous coronary intervention, coronary artery bypass grafting). In some embodiments, the subject in need thereof is undergoing an organ transplantation procedure (e.g., skin, kidney, liver, heart, lung). In some embodiments, the subject is undergoing cosmetic dermatologic treatments (e.g., laser resurfacing).

In some embodiments, the pharmaceutical composition is a liquid formulation formulated for intravenous (IV) injection. In some embodiments, the composition is formulated for IV bolus injection.

In some embodiments, the pH of the pharmaceutical composition is from about 7 to about 8, for example about 7.5. In some embodiments, the pharmaceutical composition comprises in an alkalinizing agent, such as tromethamine or dibasic sodium phosphate. In some embodiments, the alkalinizing can be at a concentration of about 10 to about 50 mM, for example about 20 mM. In some embodiments, the pharmaceutical composition comprises a surfactant. In some embodiments, the surfactant is a non-ionic surfactant such as polysorbate 80 or polysorbate 20 may be present in a concentration of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% (w/v) or any concentration therebetween. In some embodiments, the pharmaceutical composition comprises sucrose. In some embodiments, the sucrose may be present in a concentration of about 1%, 2%, 3%, 4%, 5%, 6%, 7%, 8%, 9%, 10% (w/v) or any concentration therebetween.

In some embodiments, the pharmaceutical composition comprises from about 1 to about 100 g/l chimeric protein, from about 10 mM to about 50 mM tromethamine, from about 2 to about 15% (w/v) sucrose, from about 0.001% to about 0.04% (w/v) Polysorbate 80, at pH 7.5.

In some embodiments, the chimeric protein (from about 1 to about 100 g/1) is formulated in about 20 mM tromethamine, about 7.5% sucrose, and about 0.02% polysorbate 80 at pH 7.5 (via addition of HCl). In some embodiments, about 20 mg/ml of the chimeric protein is formulated in about 20 mM tromethamine, about 7.5% sucrose, and about 0.02% polysorbate 80 at pH 7.5 (via addition of HCl).

In some embodiments, the pH of the pharmaceutical composition is from about 4.5 to about 6.5, from about 4.5 to about 5, from about 5 to about 5.5, from about 5.5 to about 6, from about 6 to about 6.5, about 4.5, about 5, about 5.5, about 6, about 6.6. In some embodiments, the pharmaceutical composition comprises acetate, phosphate, citrate, histidine, for example at a concentration of 10 to 50 mM. In some embodiments, the pharmaceutical composition comprises acetate. In some embodiments, the citrate can be at a concentration of about 10 to about 50 mM, for example about 20 mM. In some embodiments, the pharmaceutical composition comprises sucrose. In some embodiments, the sucrose may be present in a concentration of about 1%, 2%, 3%, 4%, 5%, 6%, 7%, 8%, 9%, 10% or any concentration therebetween (w/v).

In some embodiments, the pharmaceutical composition comprising the chimeric protein comprises citrate, sucrose or a combination thereof.

In some embodiments, the pharmaceutical composition comprises less than 2% (w/v) sucrose, less than 1.5% (w/v) sucrose, less than 1% (w/v) sucrose, less 0.5% (w/v) sucrose, less than 0.1% (w/v) sucrose, less than 0.01% (w/v) sucrose. In some embodiments, the pharmaceutical composition is free of sucrose.

In some embodiments, the pharmaceutical composition comprises from about 1 to about 25 g/l chimeric protein or any value therebetween.

In some embodiments, the pharmaceutical composition has a pH of from about 5 to about 7, for example about 5, about 5.5, about 6, about 6.5, about 7.

In some embodiments, the pharmaceutical composition comprises from about 100 mM to about 150 mM sodium chloride and sterile water. In some embodiments, the pharmaceutical composition is a preservative-free formulation. In some embodiments, the pharmaceutical composition is sterile.

In some embodiments, the pharmaceutical composition comprises excipients that are such as, but not limited, to histidine, histidine monohydrochloride, histidine hydrochloride, methionine, succinic acid, trehalose dihydrate, polysorbate 20, and polysorbate 80.

In some embodiments, the pharmaceutical composition has an osmolality of about 45-140 mOsmol/kg, or any value therebetween, for example 45-75 mOsmol/kg, 74-110 mOsmol/kg or 110-140 mOsmol/kg.

In some embodiments, the pharmaceutical composition comprises a therapeutically effective amount of the chimeric protein, about 20 mM sodium citrate, about 122 mM sodium chloride and has a pH of about 6.5. In some embodiments, the pharmaceutical composition consists essentially of a therapeutically effective amount of the chimeric protein, about 20 mM sodium citrate, about 122 mM sodium chloride and has a pH of about 6.5. In some embodiments, the pharmaceutical composition consists of a therapeutically effective amount of the chimeric protein, about 20 mM sodium citrate, about 122 mM sodium chloride and has a pH of about 6.5.

In some embodiments, the pharmaceutical composition comprises from about 1 to about 25 g/l chimeric protein, from about 2 to about 15% (w/v) sucrose, at pH 5.

In some embodiments, the chimeric protein (from about 1 to about 10 g/l) is formulated in about 20 mM citrate, about 7.5% sucrose, at pH 5. In some embodiments, about 5 mg/ml of the chimeric protein is formulated in about 20 mM citrate, about 7.5% sucrose, at pH 5.

In some embodiments, the pharmaceutical composition comprising the chimeric protein comprises alkalinizing agent, a surfactant, sucrose or a combination thereof.

The pharmaceutical compositions of the disclosure may contain pharmaceutically acceptable auxiliary substances as required to approximate physiological conditions, such as pH adjusting and buffering agents, tonicity adjusting agents, wetting agents, detergents, preservatives and the like. In some embodiments, the liquid composition is sterilized by conventional sterilization techniques, or sterile filtered. In some embodiments, the liquid composition is in a vial.

In some embodiments, the pharmaceutical composition is administered intravenously or intraarterially to a subject in need thereof via bolus injection. A bolus injection comprises, e.g., fast intravenous injection, for example less than 10 seconds (or less than 20, 30, 40, 50, 60 second), or intravenous infusion over less than approximately 3 minutes, 4 minutes, 5 minutes, 6 minutes, 7 minutes, 8 minutes, 9 minutes or 10 minutes. In some embodiments, the pharmaceutical composition is administered intravenously to a subject in need thereof. In some embodiments, the composition is administered as an I.V. push. In some embodiments, the administration is over 5 min, or less, 4 min, or less, 3 min. of less, 2 min. or less, 1 min or less (for example 50 s, 40 s, 30 s, 20 s or any administration time therebetween). In other embodiments, the composition is administered as a slow I.V. injection.

In some embodiments, the pharmaceutical composition is administered intravenously to a subject using a syringe injection pump at a rate of 0.5-25 mL/min. For example, the pharmaceutical composition is administered intravenously to a subject using a syringe injection pump at a rate of 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5 or 25 mL/min. In some embodiments, the pharmaceutical composition is administered intravenously to a subject using a syringe injection pump at a rate of 0.5-1, 1-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.5-8, 8-8.5, 8.5-9, 9-9.5, 9.5-10, 10-10.5, 10.5-11, 11-11.5, 11.5-12, 12-12.5, 12.5-13, 13-13.5, 13.5-14, 14-14.5, 14.5-15, 15-15.5, 15.5-16, 16-16.5, 16.5-17, 17-17.5, 17.5-18, 18-18.5, 18.5-19, 19-19.5, 19.5-20, 20-20.5, 20.5-21, 21-21.5, 21.5-22, 22-22.5, 22.5-23, 23-23.5, 23.5-24, 24-24.5 or 24.5-25 mL/min

In some embodiments, the pharmaceutical composition is administered intraarterially to a subject using a syringe injection pump at a rate of 0.05-10 mL/min. For example, the pharmaceutical composition is administered intraarterially to a subject using a syringe injection pump at a rate of 0.05, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or 10 mL/min. In some embodiments, the pharmaceutical composition is administered intraarterially to a subject using a syringe injection pump at a rate of 0.05-0.1, 0.1-0.2, 0.2-0.3, 0.3-0.4, 0.4-0.5, 0.5-0.6, 0.6-0.7, 0.7-0.8, 0.8-0.9, 0.9-1, 1-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.5-8, 8-8.5, 8.5-9, 9-9.5, 9.5-10 mL/min.

In some embodiments, the pharmaceutical composition is administered intrathecally to a subject using techniques known in the art.

In some embodiments, a therapeutically effective amount generally is in the range of about 0.01 mg/kg to about 100.0 mg/kg per dose. In some embodiments, a therapeutically effective amount of a protein disclosed herein ranges from about 0.01 mg/kg to about 0.05 mg/kg per dose, about 0.01 mg/kg to about 0.1 mg/kg per dose, 0.01 mg/kg to about 1 mg/kg per dose, 0.01 mg/kg to about 10 mg/kg per dose, 0.01 mg/kg to about 100.0 mg/kg per dose, 0.1 mg/kg to about 0.5 mg/kg per dose, 0.1 mg/kg to about 1 mg/kg per dose, 0.1 mg/kg to about 5 mg/kg per dose, 0.1 mg/kg to about 10 mg/kg per dose, 0.1 mg/kg to about 20 mg/kg per dose, 0.1 mg/kg to about 30 mg/kg per dose, 0.1 mg/kg to about 40 mg/kg per dose, 0.1 mg/kg to about 50 mg/kg per dose, 0.1 mg/kg to about 60 mg/kg per dose, 0.1 mg/kg to about 70 mg/kg per dose, 0.1 mg/kg to about 80 mg/kg per dose, 0.1 mg/kg to about 90 mg/kg per dose, 0.1 mg/kg to about 100 mg/kg per dose, 1 mg/kg to about 5 mg/kg per dose, 1 mg/kg to about 10 mg/kg per dose, 1 mg/kg to about 20 mg/kg per dose, 1 mg/kg to about 30 mg/kg per dose, 1 mg/kg to about 40 mg/kg per dose, 1 mg/kg to about 50 mg/kg per dose, 1 mg/kg to about 60 mg/kg per dose, 1 mg/kg to about 70 mg/kg per dose, 1 mg/kg to about 80 mg/kg per dose, 1 mg/kg to about 90 mg/kg per dose, 1 mg/kg to about 100 mg/kg per dose, 10 mg/kg to about 20 mg/kg per dose, 10 mg/kg to about 30 mg/kg per dose, 10 mg/kg to about 40 mg/kg per dose, 10 mg/kg to about 50 mg/kg per dose, 10 mg/kg to about 60 mg/kg per dose, 10 mg/kg to about 70 mg/kg per dose, 10 mg/kg to about 80 mg/kg per dose, 10 mg/kg to about 90 mg/kg per dose, or 10 mg/kg to about 100 mg/kg per dose.

In some embodiments, a therapeutically effective amount generally is in the range of about 0.01 mg/kg to about 20.0 mg/kg per dose. In some embodiments, a therapeutically effective amount ranges from about 0.01 mg/kg to about 10.0 mg/kg per dose.

In some embodiments, a therapeutically effective amount of a protein disclosed herein ranges from about 0.01 mg/kg to about 0.02 mg/kg per dose, about 0.01 mg/kg to about 0.03 mg/kg per dose, about 0.01 mg/kg to about 0.04 mg/kg per dose, about 0.01 mg/kg to about 0.05 mg/kg per dose, about 0.01 mg/kg to about 0.06 mg/kg per dose, about 0.01 mg/kg to about 0.07 mg/kg per dose, about 0.01 mg/kg to about 0.08 mg/kg per dose, about 0.01 mg/kg to about 0.09 mg/kg per dose, about 0.01 mg/kg to about 0.1 mg/kg per dose, about 0.01 mg/kg to about 0.2 mg/kg per dose, about 0.01 mg/kg to about 0.3 mg/kg per dose, about 0.01 mg/kg to about 0.4 mg/kg per dose, about 0.01 mg/kg to about 0.5 mg/kg per dose, about 0.01 mg/kg to about 0.6 mg/kg per dose, about 0.01 mg/kg to about 0.7 mg/kg per dose, about 0.01 mg/kg to about 0.8 mg/kg per dose, about 0.01 mg/kg to about 0.9 mg/kg per dose, about 0.01 mg/kg to about 1 mg/kg per dose, about 0.01 mg/kg to about 2 mg/kg per dose, about 0.01 mg/kg to about 3 mg/kg per dose, about 0.01 mg/kg to about 4 mg/kg per dose, about 0.01 mg/kg to about 5 mg/kg per dose, about 0.01 mg/kg to about 6 mg/kg per dose, about 0.01 mg/kg to about 7 mg/kg per dose, about 0.01 mg/kg to about 8 mg/kg per dose, about 0.01 mg/kg to about 9 mg/kg per dose, about 0.01 mg/kg to about 10 mg/kg per dose, about 0.01 mg/kg to about 11 mg/kg per dose, about 0.01 mg/kg to about 12 mg/kg per dose, about 0.01 mg/kg to about 13 mg/kg per dose, about 0.01 mg/kg to about 14 mg/kg per dose, about 0.01 mg/kg to about 15 mg/kg per dose, about 0.01 mg/kg to about 16 mg/kg per dose, about 0.01 mg/kg to about 17 mg/kg per dose, about 0.01 mg/kg to about 18 mg/kg per dose, about 0.01 mg/kg to about 19 mg/kg per dose, about 0.01 mg/kg to about 20 mg/kg per dose.

In some embodiments, a therapeutically effective amount of a protein disclosed herein ranges from about 0.01 mg/kg to about 0.05 mg/kg per dose, about 0.05 mg/kg to about 0.1 mg/kg per dose, about 0.1 mg/kg to about 0.5 mg/kg per dose, about 0.05 mg/kg to about 1 mg/kg per dose, about 1 mg/kg to about 2 mg/kg per dose, about 2 mg/kg to about 3 mg/kg per dose, about 3 mg/kg to about 4 mg/kg per dose, about 4 mg/kg to about 5 mg/kg per dose, about 5 mg/kg to about 6 mg/kg per dose, about 6 mg/kg to about 7 mg/kg per dose, about 7 mg/kg to about 8 mg/kg per dose, about 8 mg/kg to about 9 mg/kg per dose, about 9 mg/kg to about 10 mg/kg per dose, about 10 mg/kg to about 11 mg/kg per dose, about 11 mg/kg to about 12 mg/kg per dose, about 12 mg/kg to about 13 mg/kg per dose, about 13 mg/kg to about 14 mg/kg per dose, about 14 mg/kg to about 15 mg/kg per dose, about 15 mg/kg to about 16 mg/kg per dose, about 16 mg/kg to about 17 mg/kg per dose, 17 mg/kg to about 18 mg/kg per dose, about 18 mg/kg to about 19 mg/kg per dose, about 19 mg/kg to about 20 mg/kg per dose.

In some embodiments, a therapeutically effective amount of a protein disclosed herein is about 0.01 mg/kg per dose, about 0.02 mg/kg per dose, about 0.03 mg/kg per dose, about 0.04 mg/kg per dose, about 0.05 mg/kg per dose, about 0.06 mg/kg per dose, about 0.07 mg/kg per dose, about 0.08 mg/kg per dose, about 0.09 mg/kg per dose, about 0.1 mg/kg per dose, about 0.2 mg/kg per dose, about 0.3 mg/kg per dose, about 0.4 mg/kg per dose, about 0.5 mg/kg per dose, about 0.6 mg/kg per dose, about 0.7 mg/kg per dose, about 0.8 mg/kg per dose, about 0.9 mg/kg per dose, about 1 mg/kg per dose, about 1.1 mg/kg per dose, about 1.2 mg/kg per dose, about 1.3 mg/kg per dose, about 1.4 mg/kg per dose, about 1.5 mg/kg per dose, about 1.6 mg/kg per dose, about 1.7 mg/kg per dose, about 1.8 mg/kg per dose, about 1.9 mg/kg per dose, about 2 mg/kg per dose, about 2.1 mg/kg per dose, about 2.2 mg/kg per dose, about 2.3 mg/kg per dose, about 2.4 mg/kg per dose, about 2.5 mg/kg per dose, about 2.6 mg/kg per dose, about 2.7 mg/kg per dose, about 2.8 mg/kg per dose, about 2.9 mg/kg per dose, about 3 mg/kg per dose, about 3.1 mg/kg per dose, about 3.2 mg/kg per dose, about 3.3 mg/kg per dose, about 3.4 mg/kg per dose, about 3.5 mg/kg per dose, about 3.6 mg/kg per dose, about 3.7 mg/kg per dose, about 3.8 mg/kg per dose, about 3.9 mg/kg per dose, about 4 mg/kg per dose, about 4.1 mg/kg per dose, about 4.2 mg/kg per dose, about 4.3 mg/kg per dose, about 4.4 mg/kg per dose, about 4.5 mg/kg per dose, about 4.6 mg/kg per dose, about 4.7 mg/kg per dose, about 4.8 mg/kg per dose, about 4.9 mg/kg per dose, about 5 mg/kg per dose, about 5.1 mg/kg per dose, about 5.2 mg/kg per dose, about 5.3 mg/kg per dose, about 5.4 mg/kg per dose, about 5.5 mg/kg per dose, about 5.6 mg/kg per dose, about 5.7 mg/kg per dose, about 5.8 mg/kg per dose, about 5.9 mg/kg per dose, about 6 mg/kg per dose, about 6.1 mg/kg per dose, about 6.2 mg/kg per dose, about 6.3 mg/kg per dose, about 6.4 mg/kg per dose, about 6.5 mg/kg per dose, about 6.6 mg/kg per dose, about 6.7 mg/kg per dose, about 6.8 mg/kg per dose, about 6.9 mg/kg per dose, about 7 mg/kg per dose, about 7.1 mg/kg per dose, about 7.2 mg/kg per dose, about 7.3 mg/kg per dose, about 7.4 mg/kg per dose, about 7.5 mg/kg per dose, about 7.6 mg/kg per dose, about 7.7 mg/kg per dose, about 7.8 mg/kg per dose, about 7.9 mg/kg per dose, about 8 mg/kg per dose, about 8.1 mg/kg per dose, about 8.2 mg/kg per dose, about 8.3 mg/kg per dose, about 8.4 mg/kg per dose, about 8.5 mg/kg per dose, about 8.6 mg/kg per dose, about 8.7 mg/kg per dose, about 8.8 mg/kg per dose, about 8.9 mg/kg per dose, about 9 mg/kg per dose, about 9.1 mg/kg per dose, about 9.2 mg/kg per dose, about 9.3 mg/kg per dose, about 9.4 mg/kg per dose, about 9.5 mg/kg per dose, about 9.6 mg/kg per dose, about 9.7 mg/kg per dose, about 9.8 mg/kg per dose, about 9.9 mg/kg per dose, about 10 mg/kg per dose, about 11 mg/kg per dose, about 12 mg/kg per dose, about 13 mg/kg per dose, about 14 mg/kg per dose, about 15 mg/kg per dose, about 16 mg/kg per dose, about 17 mg/kg per dose, about 18 mg/kg per dose, about 19 mg/kg per dose, about 20 mg/kg per dose.

In some embodiments, a therapeutically effective amount generally is in the range of about 1 mg/kg to about 10.0 mg/kg per dose. In some embodiments, an effective amount of a protein disclosed herein can be, e.g., about 1 mg/kg to about 10 mg/kg per dose, 1 mg/kg to about 9 mg/kg per dose, 1 mg/kg to about 8 mg/kg per dose, 1 mg/kg to about 7 mg/kg per dose, 1 mg/kg to about 6 mg/kg per dose, 1 mg/kg to about 5 mg/kg per dose, 1 mg/kg to about 4 mg/kg per dose, 1 mg/kg to about 3 mg/kg per dose or 1 mg/kg to about 2 mg/kg per dose.

Dosing can be single dosage or cumulative (serial dosing), and can be readily determined by one skilled in the art. For instance, treatment of a nervous system disorder may comprise a one-time administration of an effective dose of the pharmaceutical composition disclosed herein. As a non-limiting example, an effective dose of the composition disclosed herein can be administered once to a patient, e.g., as a single injection or bolus. Alternatively, treatment of a nervous system disorder may comprise multiple administrations of an effective dose of the pharmaceutical composition disclosed herein carried out over a range of time periods, such as, e.g., four times daily, three times daily, twice daily, daily, once every few days, weekly, monthly or yearly. As a non-limiting example, a combination disclosed herein can be administered once or twice weekly to a patient. The timing of administration can upon such factors as the severity of the patient's symptoms. For example, an effective dose of the composition disclosed herein can be administered to a patient once a month for an indefinite period of time, or until the mammal no longer requires therapy.

In some embodiments, a therapeutically effective amount generally is in the range of about 0.01 mg/kg to about 200.0 mg/kg per day. In some embodiments, an effective amount of a protein disclosed herein ranges from about 0.01 mg/kg to about 0.1 mg/kg per day, 0.01 mg/kg to about 1 mg/kg per day, 0.01 mg/kg to about 10 mg/kg per day, 0.01 mg/kg to about 100.0 mg/kg per day, 0.01 mg/kg to about 200.0 mg/kg per day, 0.1 mg/kg to about 1 mg/kg per day, 0.1 mg/kg to about 10 mg/kg per day, 0.1 mg/kg to about 100 mg/kg per day, 0.1 mg/kg to about 200 mg/kg per day, 1 mg/kg to about 100 mg/kg per day, 1 mg/kg to about 200 mg/kg per day, 10 mg/kg to about 100 mg/kg per day or 10 mg/kg to about 100 mg/kg per day.

In some embodiments, a therapeutically effective amount generally is in the range of about 0.01 mg/kg to about 20.0 mg/kg per day. In some embodiments, a therapeutically effective amount generally is in the range of about 0.01 mg/kg to about 10.0 mg/kg per day. In some embodiments, a therapeutically effective amount generally is in the range of about 1 mg/kg to about 20.0 mg/kg per day. In some embodiments, a therapeutically effective amount generally is in the range of about 1 mg/kg to about 10.0 mg/kg per day. In some embodiments, an effective amount of a protein disclosed herein can be, e.g., about 1 mg/kg to about 10 mg/kg per day, 1 mg/kg to about 9 mg/kg per day, 1 mg/kg to about 8 mg/kg per day, 1 mg/kg to about 7 mg/kg per day, 1 mg/kg to about 6 mg/kg per day, 1 mg/kg to about 5 mg/kg per day, 1 mg/kg to about 4 mg/kg per day, 1 mg/kg to about 3 mg/kg per day or 1 mg/kg to about 2 mg/kg per day.

In some embodiments, a therapeutically effective amount of a chimeric protein disclosed herein ranges from about 0.01 mg/kg to about 0.02 mg/kg per day, about 0.01 mg/kg to about 0.03 mg/kg per day, about 0.01 mg/kg to about 0.04 mg/kg per day, about 0.01 mg/kg to about 0.05 mg/kg per day, about 0.01 mg/kg to about 0.06 mg/kg per day, about 0.01 mg/kg to about 0.07 mg/kg per day, about 0.01 mg/kg to about 0.08 mg/kg per day, about 0.01 mg/kg to about 0.09 mg/kg per day, about 0.01 mg/kg to about 0.1 mg/kg per day, about 0.01 mg/kg to about 0.2 mg/kg per day, about 0.01 mg/kg to about 0.3 mg/kg per day, about 0.01 mg/kg to about 0.4 mg/kg per day, about 0.01 mg/kg to about 0.5 mg/kg per day, about 0.01 mg/kg to about 0.6 mg/kg per day, about 0.01 mg/kg to about 0.7 mg/kg per day, about 0.01 mg/kg to about 0.8 mg/kg per day, about 0.01 mg/kg to about 0.9 mg/kg per day, about 0.01 mg/kg to about 1 mg/kg per day, about 0.01 mg/kg to about 2 mg/kg per day, about 0.01 mg/kg to about 3 mg/kg per day, about 0.01 mg/kg to about 4 mg/kg per day, about 0.01 mg/kg to about 5 mg/kg per day, about 0.01 mg/kg to about 6 mg/kg per day, about 0.01 mg/kg to about 7 mg/kg per day, about 0.01 mg/kg to about 8 mg/kg per day, about 0.01 mg/kg to about 9 mg/kg per day, about 0.01 mg/kg to about 10 mg/kg per day, about 0.01 mg/kg to about 11 mg/kg per day, about 0.01 mg/kg to about 12 mg/kg per day, about 0.01 mg/kg to about 13 mg/kg per day, about 0.01 mg/kg to about 14 mg/kg per day, about 0.01 mg/kg to about 15 mg/kg per day, about 0.01 mg/kg to about 16 mg/kg per day, about 0.01 mg/kg to about 17 mg/kg per day, about 0.01 mg/kg to about 18 mg/kg per day, about 0.01 mg/kg to about 19 mg/kg per day, or about 0.01 mg/kg to about 20 mg/kg per day.

In some embodiments, a therapeutically effective amount of a chimeric protein disclosed herein ranges from about 0.01 mg/kg to about 0.05 mg/kg per day, about 0.05 mg/kg to about 0.1 mg/kg per day, about 0.1 mg/kg to about 0.5 mg/kg per day, about 0.05 mg/kg to about 1 mg/kg per day, about 1 mg/kg to about 2 mg/kg per day, about 2 mg/kg to about 3 mg/kg per day, about 3 mg/kg to about 4 mg/kg per day, about 4 mg/kg to about 5 mg/kg per day, about 5 mg/kg to about 6 mg/kg per day, about 6 mg/kg to about 7 mg/kg per day, about 7 mg/kg to about 8 mg/kg per day, about 8 mg/kg to about 9 mg/kg per day, about 9 mg/kg to about 10 mg/kg per day, about 10 mg/kg to about 11 mg/kg per day, about 11 mg/kg to about 12 mg/kg per day, about 12 mg/kg to about 13 mg/kg per day, about 13 mg/kg to about 14 mg/kg per day, about 14 mg/kg to about 15 mg/kg per day, about 15 mg/kg to about 16 mg/kg per day, about 16 mg/kg to about 17 mg/kg per day, 17 mg/kg to about 18 mg/kg per day, about 18 mg/kg to about 19 mg/kg per day, or about 19 mg/kg to about 20 mg/kg per day.

In some embodiments, a therapeutically effective amount of a chimeric protein disclosed herein ranges about 0.01 mg/kg per day, about 0.02 mg/kg per day, about 0.03 mg/kg per day, about 0.04 mg/kg per day, about 0.05 mg/kg per day, about 0.06 mg/kg per day, about 0.07 mg/kg per day, about 0.08 mg/kg per day, about 0.09 mg/kg per day, about 0.1 mg/kg per day, about 0.2 mg/kg per day, about 0.3 mg/kg per day, about 0.4 mg/kg per day, about 0.5 mg/kg per day, about 0.6 mg/kg per day, about 0.7 mg/kg per day, about 0.8 mg/kg per day, about 0.9 mg/kg per day, about 1 mg/kg per day, about 1.1 mg/kg per day, about 1.2 mg/kg per day, about 1.3 mg/kg per day, about 1.4 mg/kg per day, about 1.5 mg/kg per day, about 1.6 mg/kg per day, about 1.7 mg/kg per day, about 1.8 mg/kg per day, about 1.9 mg/kg per day, about 2 mg/kg per day, about 2.1 mg/kg per day, about 2.2 mg/kg per day, about 2.3 mg/kg per day, about 2.4 mg/kg per day, about 2.5 mg/kg per day, about 2.6 mg/kg per day, about 2.7 mg/kg per day, about 2.8 mg/kg per day, about 2.9 mg/kg per day, about 3 mg/kg per day, about 3.1 mg/kg per day, about 3.2 mg/kg per day, about 3.3 mg/kg per day, about 3.4 mg/kg per day, about 3.5 mg/kg per day, about 3.6 mg/kg per day, about 3.7 mg/kg per day, about 3.8 mg/kg per day, about 3.9 mg/kg per day, about 4 mg/kg per day, about 4.1 mg/kg per day, about 4.2 mg/kg per day, about 4.3 mg/kg per day, about 4.4 mg/kg per day, about 4.5 mg/kg per day, about 4.6 mg/kg per day, about 4.7 mg/kg per day, about 4.8 mg/kg per day, about 4.9 mg/kg per day, about 5 mg/kg per day, about 5.1 mg/kg per day, about 5.2 mg/kg per day, about 5.3 mg/kg per day, about 5.4 mg/kg per day, about 5.5 mg/kg per day, about 5.6 mg/kg per day, about 5.7 mg/kg per day, about 5.8 mg/kg per day, about 5.9 mg/kg per day, about 6 mg/kg per day, about 6.1 mg/kg per day, about 6.2 mg/kg per day, about 6.3 mg/kg per day, about 6.4 mg/kg per day, about 6.5 mg/kg per day, about 6.6 mg/kg per day, about 6.7 mg/kg per day, about 6.8 mg/kg per day, about 6.9 mg/kg per day, about 7 mg/kg per day, about 7.1 mg/kg per day, about 7.2 mg/kg per day, about 7.3 mg/kg per day, about 7.4 mg/kg per day, about 7.5 mg/kg per day, about 7.6 mg/kg per day, about 7.7 mg/kg per day, about 7.8 mg/kg per day, about 7.9 mg/kg per day, about 8 mg/kg per day, about 8.1 mg/kg per day, about 8.2 mg/kg per day, about 8.3 mg/kg per day, about 8.4 mg/kg per day, about 8.5 mg/kg per day, about 8.6 mg/kg per day, about 8.7 mg/kg per day, about 8.8 mg/kg per day, about 8.9 mg/kg per day, about 9 mg/kg per day, about 9.1 mg/kg per day, about 9.2 mg/kg per day, about 9.3 mg/kg per day, about 9.4 mg/kg per day, about 9.5 mg/kg per day, about 9.6 mg/kg per day, about 9.7 mg/kg per day, about 9.8 mg/kg per day, about 9.9 mg/kg per day, about 10 mg/kg per day, about 11 mg/kg per day, about 12 mg/kg per day, about 13 mg/kg per day, about 14 mg/kg per day, about 15 mg/kg per day, about 16 mg/kg per day, about 17 mg/kg per day, about 18 mg/kg per day, about 19 mg/kg per day, or about 20 mg/kg per day.

In some embodiments, the effective dose is administered daily to the subject in need thereof over a period of 2 days, 3 days, 4 days, 5 days, 6 days, 7 days, 8 days, 9 days, 10 days, 11 days, 12 days, 13 days, 14 days or more. In some embodiments, the effective dose is administered daily to the subject having an acute CNS injury over a period of a minimum 2 days to a period of 14 days, for example 4, 5, 6, 7 days. In some embodiments, the effective dose is administered daily to the subject having an acute cardiovascular injury (e.g., STEMI, Cardiac Arrest) over a period of a minimum 2 days to a period of 14 days, for example 4, 5, 6, 7 days. In some embodiments, the effective dose is administered daily to the subject having an acute kidney injury over a period of a minimum 2 days to a period of 14 days, for example 4, 5, 6, 7 days. In some embodiments, the effective dose is administered daily to the subject experiencing acute radiation sickness (e.g., GI-ARS, H-ARS) over a period of a minimum 2 days to a period of 14 days, for example 4, 5, 6, 7 days. In some embodiments, the effective dose is administered daily to the subject experiencing chemical inhalation injury (e.g., sulfur mustards exposure) over a period of a minimum 2 days to a period of 14 days, for example 4, 5, 6, 7 days. In some embodiments, the effective dose is administered daily to the subject undergoing a procedure with risk of iatrogenic injury (e.g., transcatheter aortic valve replacement, percutaneous coronary intervention, coronary artery bypass grafting) over a period of a minimum 1 day to a period of 14 days, for example 4, 5, 6, 7 days. In some embodiments, the effective dose is administered to the subject undergoing an organ transplantation procedure (e.g., skin, kidney, liver, heart, lung) over a period of a minimum 1 day to a period of 14 days, for example 4, 5, 6, 7 days. In some embodiments, the effective dose is administered to the subject undergoing cosmetic dermatologic treatments (e.g., laser resurfacing) over a period of a minimum 1 day to a period of 14 days, for example 4, 5, 6, 7 days. In some embodiments, the effective dose is administered to the subject being treated for dermatologic injuries (e.g., wound healing) over a period of a minimum 1 day to a period of 14 days, for example 4, 5, 6, 7 days. In some embodiments, the effective dose is administered to the subject being treated for traumatic injuries (e.g., burn, crush, laceration, contusion, avulsion, concussion, fracture, amputation) over a period of a minimum 1 day to a period of 14 days, for example 4, 5, 6, 7 days. In some embodiments, the effective dose is administered to the subject experiencing chronic neurodegenerative diseases (e.g., synucleinopathies, amyloidoses) in an intermittent repeat dosing regimen as a life-prolonging treatment.

In some embodiments, the effective dose is a descending regimen that is sequentially administered daily to the subject in need thereof over a period of 2 days, 3 days, 4 days, 5 days, 6 days, 7 days, 8 days, 9 days, 10 days, 11 days, 12 days, 13 days, 14 days or more. In some embodiments, descending regimen decreases the requirement for the supplemental dextrose infusion.

In some embodiments, the effective dose is administered daily to the subject having neurovegetative disease over a period of a minimum 2 days to at least 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 11 years, 12, years, 13 years, 14 years, 15 years, 16, years, 17 years, 18 years, 19 years or 20 years.

In some embodiments, the effective amount is administered once a day (or every 24 hours) to the subject in need thereof. In some embodiments, the effective amount is administered once a day to the subject in need thereof over a period of 2 days, 3 days, 4 days, 5 days, 6 days, 7 days, 8 days, 9 days, 10 days, 11 days, 12 days, 13 days, 14 days or more. In some embodiments, the effective amount is administered once a day to the subject in need thereof over a period of up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days. In some embodiments, the effective amount is administered once a day to the subject in need thereof over a period of 7 days. In some embodiments, the effective amount is administered two, three or more times a day to the subject in need thereof. In some embodiments, the effective amount is administered two, three or more times a day to the subject in need thereof over a period of 2 days, 3 days, 4 days, 5 days, 6 days, 7 days, 8 days, 9 days, 10 days, 11 days, 12 days, 13 days, 14 days or more. In some embodiments, the effective amount is administered two, three or more times a day to the subject in need thereof over a period of up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days. In some embodiments, the effective amount is administered two, three or more times a day to the subject in need thereof over a period of 7 days.

In some embodiments, a total dose of about 5 to 100 mg/kg of the chimeric proteins described herein is administered over a period of 4 days, 5 days, 6 days, or 7 days. In some embodiments, a total dose of about 5 to 100 mg/kg of the chimeric proteins described herein is administered over a period of 7 days. In some embodiments, a total dose of about 5 to 90 mg/kg of the chimeric proteins described herein is administered over a period of 4 days, 5 days, 6 days, or 7 days. In some embodiments, a total dose of about 5 to 90 mg/kg of the chimeric proteins described herein is administered over a period of 7 days. In some embodiments, a total dose of about 5 to 80 mg/kg of the chimeric proteins described herein is administered over a period of 4 days, 5 days, 6 days, or 7 days. In some embodiments, a total dose of about 5 to 80 mg/kg of the chimeric proteins described herein is administered over a period of 7 days. In some embodiments, a total dose of about 5 to 70 mg/kg of the chimeric proteins described herein is administered over a period of 4 days, 5 days, 6 days, or 7 days. In some embodiments, a total dose of about 5 to 70 mg/kg of the chimeric proteins described herein is administered over a period of 7 days. In some embodiments, a total dose of about 5 to 60 mg/kg of the chimeric proteins described herein is administered over a period of 4 days, 5 days, 6 days, or 7 days. In some embodiments, a total dose of about 5 to 60 mg/kg of the chimeric proteins described herein is administered over a period of 7 days. In some embodiments, a total dose of about 5 to 50 mg/kg of the chimeric proteins described herein is administered over a period of 4 days, 5 days, 6 days, or 7 days. In some embodiments a total dose of about 5 to 50 mg/kg of the chimeric proteins described herein is administered over a period of 7 days. In some embodiments, a total dose of about 5 to 40 mg/kg of the chimeric proteins described herein is administered over a period of 4 days, 5 days, 6 days, or 7 days. In some embodiments, a total dose of about 5 to 40 mg/kg of the chimeric proteins described herein is administered over a period of 7 days. In some embodiments, a total dose of about 5 to 30 mg/kg of the chimeric proteins described herein is administered over a period of 4 days, 5 days, 6 days, or 7 days. In some embodiments, a total dose of about 5 to 30 mg/kg of the chimeric proteins described herein is administered over a period of 7 days. In some embodiments, a total dose of about 5 to 20 mg/kg of the chimeric proteins described herein is administered over a period of 4 days, 5 days, 6 days, or 7 days. In some embodiments, a total dose of about 5 to 20 mg/kg of the chimeric proteins described herein is administered over a period of 7 days. In some embodiments, a total dose of about 5 to 20 mg/kg of the chimeric proteins described herein is administered over a period of 4 days.

In some embodiments, a total dose of about 100 to 500 mg/kg of the chimeric proteins described herein is administered over a period of 4 days, 5 days, 6 days, or 7 days. In some embodiments, a total dose of about 100 to 500 mg/kg of the chimeric proteins described herein is administered over a period of 7 days. In some embodiments, a total dose of about 100 to 400 mg/kg of the chimeric proteins described herein is administered over a period of 4 days, 5 days, 6 days, or 7 days. In some embodiments a total dose of about 100 to 400 mg/kg of the chimeric proteins described herein is administered over a period of 7 days. In some embodiments, a total dose of about 100 to 300 mg/kg of the chimeric proteins described herein is administered over a period of 4 days, 5 days, 6 days, or 7 days. In some embodiments, a total dose of about 100 to 300 mg/kg of the chimeric proteins described herein is administered over a period of 7 days. In some embodiments, a total dose of about 100 to 200 mg/kg of the chimeric proteins described herein is administered over a period of 4 days, 5 days, 6 days, or 7 days. In some embodiments, a total dose of about 100 to 200 mg/kg of the chimeric proteins described herein is administered over a period of 7 days. In some embodiments, a total dose of about 100 to 150 mg/kg of the chimeric proteins described herein is administered over a period of 4 days, 5 days, 6 days, or 7 days. In some embodiments, a total dose of about 100 to 150 mg/kg of the chimeric proteins described herein is administered over a period of 7 days. In some embodiments, a total dose of about 140 mg/kg of the chimeric proteins described herein is administered over a period of 4 days, 5 days, 6 days, or 7 days. In some embodiments, a total dose of about 140 mg/kg of the chimeric proteins described herein is administered over a period of 7 days.

In some embodiments, the treatment regimen comprises a descending dosage regimen. In descending dosage regimen, the amount of the dose of the chimeric protein of the disclosure administered in the subject is reduced over the course of the duration of the treatment. In some embodiments, the dose administered on the second day is lower than the dose administered the second day. In other embodiments, the dose administered on the second day is the same than the dose administered the second day, and the dose administered the third day is lower than the dose administered the second day. In some embodiments, the treatment regimen provides for a total dose of about 2 mg/kg to about 200 mg/kg, of about 2 mg/kg to about 20 mg/kg, of about 2 mg/kg to about 10 mg/kg, of about 100 mg/kg to about 200 mg/kg, of about 100 mg/kg to about 150 mg/kg over a predetermined period of time (e.g. 4, 5, 6, or 7 days). In some embodiments, the treatment regimen provides for a total dose of about 5 mg/kg, of about 10 mg/kg, or of about 20 mg/kg over a predetermined period of time (e.g. 4, 5, 6, or 7 days).

In some embodiments, a first daily dose is administered the first day, a second daily dose that comprises from about 85% to about 95% of the amount of the chimeric protein present in the first dose is administered on day 2, a third daily dose that comprises from about 65% to about 85% of the amount of the chimeric protein present in the second dose and an amount that is lower than the second dose is administered on day 3, a fourth dose that comprises from about 45% to about 65% of the amount of the chimeric protein present in the first dose and an amount that lower than the third dose is administered on day 4, a fifth dose that comprises from about 35% to about 45% of the amount of the chimeric protein present in the first dose and an amount that lower than the fourth dose is administered on day 5, a sixth dose that comprises from about 25% to about 35% of the amount of the chimeric protein present in the first dose and an amount that lower than the fifth dose is administered on day 6, and a seventh dose that comprises from about 15% to about 25% of the amount of the chimeric protein present in the first dose and an amount that lower than the sixth dose is administered on day 7. In some embodiments, the descending treatment regimen comprises administering a first daily dose on day 1, a second daily dose corresponding to about 90% of the first daily dose on day 2, a third daily dose corresponding to about 70% of the first daily dose on day 3, a fourth daily dose corresponding to about 50% of the first daily dose on day 4, a fifth daily dose corresponding to about 40% of the first daily dose on day 5, a sixth daily dose corresponding to about 30% of the first daily dose on day 6 and a seventh daily dose corresponding to about 20% of the first daily dose on day 7.

In some embodiments, a first daily dose is administered the first day, a second daily dose that comprises from about 85% to about 95% (e.g. about 90%) of the amount of the chimeric protein present in the first dose is administered on day 2, a third daily dose that comprises from about 65% to about 85% (e.g. about 70%) of the amount of the chimeric protein present in the second dose and an amount that is lower than the second dose is administered on day 3, a fourth dose that comprises from about 45% to about 65% (e.g. about 50%) of the amount of the chimeric protein present in the first dose and an amount that lower than the third dose is administered on day 4.

In some embodiments, a first daily dose administered the first day and the second daily dose administered on the second day are the same, a third daily dose comprising from about 65% to about 90% (e.g. about 75%) of the amount of the chimeric protein present in the first dose is administered on day 3, a fourth daily dose that comprises from about 45% to about 65% (e.g. about 50%) of the amount of the chimeric protein present in the second dose and an amount that is lower than the second dose is administered on day 4.

In some embodiments, the treatment regimen or descending treatment regimen provides a total dose of from about 2 mg/kg to about 20 mg/kg over a period of 7 days. In some embodiments, the treatment regimen provides a total dose of from about 2 mg/kg to about 20 mg/kg over a period of 4 days. In some embodiments, the treatment regimen or descending treatment regimen provides a total dose of from about 2 mg/kg to about 10 mg/kg over a period of 7 days. In some embodiments, the treatment regimen or descending treatment regimen provides a total dose of from about 2 mg/kg to about 10 mg/kg over a period of 4 days. In some embodiments, the treatment regimen or descending treatment regimen provides a total dose of about 5 mg/kg, about 10 mg/kg, or about 20 mg/kg over a period of 7 days. In some embodiments, the treatment regimen or descending treatment regimen provides a total dose of about 5 mg/kg, about 10 mg/kg, or about 20 mg/kg over a period of 4 days.

In some embodiments, the treatment regimen comprises a 5 day course of intravenous administration of the chimeric protein, such as scp776. In some embodiments, the treatment regimen comprises a 7 day course of intravenous administration of the chimeric protein, such as scp776. In some embodiments, the treatment regimen comprises a 4 day course of intravenous administration of the chimeric protein, such as scp776. In some embodiments, the treatment regimen comprises a 3 day course of intravenous administration of the chimeric protein, such as scp776. In some embodiments, the treatment regimen comprises a 2 day course of intravenous administration of the chimeric protein, such as scp776. In some embodiments, the treatment regimen comprises administering intravenously a first dose at from about 2 mg/kg to about 6 mg/kg on day 1, and one dose of about 1 mg/kg to about 2 mg/kg one each of the following days. In some embodiments, the first and second dose are the same (e.g. 2 mg/kg), and the third dose is lower than the first dose. For example, the first dose can be 2 mg/kg, the second dose can be 2 mg/kg, the third dose can be 1.5 mg/kg and the fourth dose can be 1 mg/kg, in a 4 day treatment course. In some embodiments, the second dose is lower than the first dose and the third dose is lower than the second dose etc., For example, the first dose can be 2 mg/kg, the second dose can be 1.8 mg/kg, the third dose can be 1.4 mg/kg and the fourth dose can be 1 mg/kg, in a 4 days treatment course. In some embodiments, the second dose is lower than the first dose and the third dose is the same as the second dose, etc., For example, the course can comprise a first dose of about 5.2 mg/kg on day 1, and one dose of about 1.3 mg/kg on day 2, days 3, day 4 and day 5, if the course is a 5 day course.

In some embodiments, the course of treatment comprises administration of the effective amount over 2 days, 3 days, 4 days, 5 days, 6 days, 7 days, 8 days, 9 days, 10 days, 11 days, 12 days, 13 days, 14 days or more. In some embodiments, the course of treatment comprises administration of the effective amount over 7 days. In some embodiments, the course of treatment comprises administration of the effective amount over 4 days. In some embodiments, the course of treatment comprises administration of the effective amount over consecutive days. In some embodiments, the course of treatment comprises administration of the effective amount every day, every 2nd day, every 3rd day or every 4th day.

In some embodiments, the human dose regimen can be calculated based on allometric scaling of the Non-Human Primate to Human dose regimen.

An estimate of the human equivalent dose (HED) to the efficacious dosing regimen can be obtained via allometric scaling (USDHHS, FDA, CDER, Guidance for Industry, 2005). Allometric scaling treats the problem of differential metabolic rates between species by applying a correction factor based on body surface area to the dose of interest in a given species. Allometric scaling is most frequently used in estimating safe starting doses for first-in-human studies, but it is also commonly applied in translating effective doses from animals to man. The FDA-recommended correction factor for converting dosages in 3 kg rhesus monkeys to humans is 3.1 (USDHHS, FDA, CDER, Guidance for Industry, 2005). In some embodiments, the estimated HED of the efficacious dose regimen comprises an initial dose of 5.2 mg/kg, followed by additional doses at 24 hour intervals of 1.3 mg/kg.

In some embodiments, the pharmaceutical compositions described herein can further include one or more additional bioactive or therapeutic agents or components to aid in the treatment of damaged tissue or cells and/or facilitate the tissue regenerative process.

In some embodiments, the method comprises administering two or more (e.g. two, there or more) pharmaceutical compositions. The different pharmaceutical compositions may be administered to the subject in any order and in any suitable interval. For example, in some embodiments, the one or more some embodiments compositions are administered simultaneously or near simultaneously. In some embodiments, the method comprises a staggered administration of the two or more some embodiments compositions, where a first some embodiments composition is administered and a second some embodiments composition administered at some later time point. Any suitable interval of administration which produces the desired therapeutic effect may be used.

In certain embodiments, the method has an additive effect, wherein the overall effect of the administering a combination of therapeutic agents or procedures is approximately equal to the sum of the effects of administering each therapeutic agent or procedure alone. In other embodiments, the method has a synergistic effect, wherein the overall effect of administering a combination of therapeutic agents or procedures is greater than the sum of the effects of administering each therapeutic agent or procedure alone.

In some embodiments, a therapeutically effective amount of the pharmaceutical composition comprising the chimeric protein reduces at least one symptom associated with a disorder by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein reduces at least one symptom associated with a nervous system disorder by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein disclosed herein reduces at least one symptom associated with a nervous system disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In some embodiments, a therapeutically effective amount of the chimeric protein reduces at least one symptom associated with a disorder for, e.g., at least one week, at least one month, at least two months, at least three months, at least four months, at least five months, at least six months, at least seven months, at least eight months, at least nine months, at least ten months, at least eleven months, or at least twelve months.

### Dextrose Supplementation

Administration of IGF-1 or IGF-1 chimeric proteins (also referred herein as chimeric protein or fusion proteins or IGF-1 containing fusion proteins) has the off-target effect of decreasing blood glucose levels. Aspects of the disclosure relate to the administration of variable rate intravenous infusion of dextrose solutions in normal saline (i.e., 0.9%), half normal saline (i.e., 0.45%), Ringer's Lactate or Hartmann's Solution (i.e., 130-131 mM NaCl, 4-5 mM KCl, 2-3 mM CaCl₂), 28-29 mM C₃H₅NaO₃), 0.2% Saline, or water to buffer against the blood glucose lowering effect. In some embodiments, euglycemic maintenance is achieved by continuous intravenous dextrose infusion following administration of IGF-1 or IGF-1 containing fusion proteins. In some embodiments, at low rates of dextrose infusion, normal blood sugar levels can be maintained following therapeutic doses of IGF-1 or IGF-1 containing fusion proteins. In some embodiments, in the event that blood glucose levels decrease following therapeutic doses of IGF-1 containing fusion proteins, transient hypoglycemia can be reversed by increasing the rate of the supplemental dextrose infusion. For subjects that receive multi-day doses of IGF-1 or IGF-1 containing fusion proteins, the rate of supplemental dextrose infusion can be slowly decreased and discontinued in the 24 to 48 hours following the last therapeutic dose of IGF-1 or IGF-1 containing fusion proteins. The euglycemic buffering potential of supplemental dextrose following administration of IGF-1 containing fusion proteins does not depend on the exact amino acid sequence of the IGF-1 variant contained within the IGF-1 containing fusion protein or the composition of other domains in the fusion protein (as the blood glucose lowering potential is attributable to the IGF-1 domain).

Provided herein are method for dextrose supplementation to support euglycemia and/or prevent prolonged hypoglycemic excursions.

In some embodiments, the method comprises administering to a subject in need thereof a dextrose solution prior to, during, and/or after administering the IGF-1 or IGF-1 chimeric protein (or the or pharmaceutical composition comprising the IGF-1 or IGF-1 chimeric protein) or any combination of the foregoing to sustain euglycemia. In some embodiments, the administration of dextrose solution mitigates the adverse hypoglycemic effect of the administration of IGF-1 or IGF-1 chimeric protein. In some embodiments, the administering of the dextrose solution is by intravenous infusion. In other embodiments, the administering of the dextrose solution is by intravenous bolus injection.

In some embodiments, the dextrose solution is administered about 30 min, about 25 min, about 20 min, about 15 min, about 10 min, about 5 min prior to administering the IGF-1 or IGF-1 chimeric protein or the pharmaceutical composition comprising the IGF-1 or IGF-1 chimeric protein. In some embodiments, the dextrose solution is administered from about 25 min to about 30 min, about 20 min to about 25 min, about 15 min to about 20 min, about 10 min to about 15 min, about 5 min to about 10 min, prior to administering the IGF-1 or IGF-1 chimeric protein or the pharmaceutical composition comprising the IGF-1 or IGF-1 chimeric protein. In some embodiments, the subject in need thereof treated with the IGF-1 or IGF-1 chimeric protein or pharmaceutical compositions described herein is continuously infused with a dextrose solution to support euglycemia. In some embodiments, the subject in need thereof is continuously infused with a dextrose solution over a period of about 24 hours, about 48 hours, about 72 hours, about 96 hours, about 120 hours. In some embodiments, the subject in need thereof is continuously infused with a dextrose solution over a period of from about 24 hours to about 120 hours, from about 24 hours to about 96 hours, from about 24 hours to about 72 hours, from about 24 hours to about 48 hours, from about 48 hours to about 120 hours, from about 48 hours to 96 hours, from about 48 hours to 72 hours, from about 72 hours to 120 hours, from about 72 hours to about 96 hours, or from about 96 hours to about 120 hours. In some embodiments, the subject in need thereof is infused with a dextrose solution about 30 min, about 25 min, about 20 min, about 15 min, about 10 min, about 5 min prior to administering the IGF-1 or IGF-1 chimeric protein and is continuously infused with a dextrose over a period of about 24 hours, about 48 hours, about 72 hours, from about 24 hours to 72 hours, from about 24 hours to 48 hours, from about 48 hours to 72 hours. In some embodiments, infusion is over a period of about 48 hours.

In some embodiments, the subject is infused with the dextrose solution for about 48 hours. In some embodiments, the subject is infused with the dextrose solution up to the end of the treatment with the IGF-1 or IGF-1 chimeric protein. In some embodiments, the subject is infused with the dextrose solution up to about 24 hours after the end of the treatment with the IGF-1 or IGF-1 chimeric protein.

For example, the subject can be treated for 4 days with the pharmaceutical composition comprising the IGF-1 or IGF-1 chimeric protein and the subject can be infused for up to 4 or 5 days with the dextrose solution. Alternatively, the subject can be treated for 1, 2, or 3 days with the pharmaceutical composition comprising the IGF-1 or IGF-1 chimeric protein and the subject can be infused for up to 2, 3, or 4 days with the dextrose solution. Alternatively, the subject can be treated daily for up to 5 days with the pharmaceutical composition comprising the IGF-1 or IGF-1 chimeric protein and the subject can be infused with the dextrose solution for up to 1 or 2 days following the last treatment of the pharmaceutical composition comprising the IGF-1 or IGF-1 chimeric protein.

In some embodiments, the subject in need thereof is administered the IGF-1 or IGF-1 chimeric protein or pharmaceutical composition comprising the IGF-1 or IGF-1 chimeric protein via bolus injection and is administered a dextrose solution by continuous intravenous infusion. In some embodiments, the subject in need thereof is administered the IGF-1 or IGF-1 chimeric protein or pharmaceutical composition comprising the IGF-1 or IGF-1 chimeric protein intrathecally and is administered a dextrose solution by continuous intravenous infusion. In some embodiments, the administering of the dextrose solution by continuous infusion is about 1 min to about 30 min prior to administering the IGF-1 or IGF-1 chimeric protein. In some embodiments, the administering of the dextrose solution by continuous infusion is about 30 min, about 25 min, about 20 min, about 15 min, about 10 min, about 5 min, about 4 min, about 3 min, about 2 min, about 1 min prior to administering the IGF-1 or IGF-1 chimeric protein. In some embodiments, the administering of the dextrose solution by continuous infusion is during the administration of the IGF-1 or IGF-1 chimeric protein or the pharmaceutical composition comprising the IGF-1 or IGF-1 chimeric protein. In some embodiments, the administering of the dextrose solution by continuous infusion is during and after the administration of the IGF-1 or IGF-1 chimeric protein or the pharmaceutical composition comprising the IGF-1 or IGF-1 chimeric protein. In some embodiments, the pharmaceutical composition comprising the IGF-1 or IGF-1 chimeric protein does not contain dextrose.

In some embodiments, the method comprises starting the dextrose infusion is started with a slow rate of infusion (i.e., 0.1 mL/kg/h) shortly before administration (t≤30 minutes) of the IGF-1 or IGF-1 containing fusion protein. In some embodiments, the method further comprises adjusting the infusions rate to support euglycemia.

In some embodiments, the infusion can be at a rate ranging from 0.01 mL/kg/hr to 5 mL/kg/hr. For example, the infusion can be at a rate of about 0.1 mL/kg/h. In some embodiments, the infusion can start at a rate of about 0.1 mL/kg/h and then be adjusted at an incremental rate of +0.05 mL/kg/hr to +2 mL/kg/hr (for example +0.05 mL/kg/hr, +0.1 mL/kg/hr, +0.2 mL/kg/hr, +0.3 mL/kg/hr, +0.4 mL/kg/hr, +0.5 mL/kg/hr, +0.6 mL/kg/hr, +0.7 mL/kg/hr, +0.8 mL/kg/hr, +0.9 mL/kg/hr, +1 mL/kg/hr, +1.1 mL/kg/hr, +1.2 mL/kg/hr, +1.3 mL/kg/hr, +1.34 mL/kg/hr, +1.5 mL/kg/hr, +1.6 mL/kg/hr, +1.7 mL/kg/hr, +1.8 mL/kg/hr, +1.9 mL/kg/hr, +2 mL/kg/hr). In some embodiments, the infusion can be at a rate of about 2 mL/kg/hr and then be adjusted at a decremental rate of-0.05 mL/kg/hr to-1 mL/kg/hr (for example −0.05 mL/kg/hr, −0.1 mL/kg/hr, −0.2 mL/kg/hr, −0.3 mL/kg/hr, −0.4 mL/kg/hr, −0.5 mL/kg/hr, −0.6 mL/kg/hr, −0.7 mL/kg/hr, −0.8 mL/kg/hr, −0.9 mL/kg/hr, −1 mL/kg/hr).

In some embodiments, the dextrose solution is in sterile water or normal saline solution. In some embodiments, the dextrose solution comprises from about 5% (w/v) dextrose to about 50% (w/v) dextrose to support euglycemia. In some embodiments, the dextrose solution is in sterile water or normal saline. In some embodiments, the subject in need thereof can be administered from 5% dextrose to 40% dextrose (w/v). In some embodiments, the subject in need thereof can be administered from 5% dextrose to 35% dextrose (w/v). In some embodiments, the subject in need thereof can be administered from 5% dextrose to 30% dextrose (w/v). In some embodiments, the subject in need thereof can be administered from 5% dextrose to 25% dextrose (w/v). In some embodiments, the subject in need thereof can be administered from 5% dextrose to 20% dextrose (w/v). In some embodiments, the subject in need thereof can be administered from 5% dextrose to 15% dextrose (w/v). In some embodiments, the subject in need thereof can be administered from 5% dextrose to 10% dextrose (w/v).

In some embodiments, the dextrose solution comprises about 5% (w/v) dextrose, about 6% (w/v) dextrose, about 7% (w/v) dextrose, about 8% (w/v) dextrose, about 9% (w/v) dextrose, about 10% (w/v) dextrose, about 11% (w/v) dextrose, about 12% (w/v) dextrose, about 13% (w/v) dextrose, about 14% (w/v) dextrose, about 15% (w/v) dextrose, about 16% (w/v) dextrose, about 17% (w/v) dextrose, about 19% (w/v) dextrose, about 20% (w/v) dextrose.

In some embodiments, the dextrose solution comprises from about 5% (w/v) dextrose to about 6% (w/v) dextrose, from about 6% (w/v) dextrose to about 7% (w/v) dextrose, from about 7% (w/v) dextrose to about 8% (w/v) dextrose, from about 8% (w/v) dextrose to about 9% (w/v) dextrose, from about 9% (w/v) dextrose to about 10% (w/v) dextrose, from about 10% (w/v) dextrose to about 11% (w/v) dextrose, from about 11% (w/v) dextrose to about 12% (w/v) dextrose, from about 12% (w/v) dextrose to about 13% (w/v) dextrose, from about 13% (w/v) dextrose to about 14% (w/v) dextrose, from about 14% (w/v) dextrose to about 15% (w/v) dextrose, from about 15% (w/v) dextrose to about 16% (w/v) dextrose, from about 16% (w/v) dextrose to about 17% (w/v) dextrose, from about 17% (w/v) dextrose to about 18% (w/v) dextrose, from about 18% (w/v) dextrose to about 19% (w/v) dextrose or from about 19% (w/v) dextrose to about 20% (w/v) dextrose.

In some embodiments, the subject in need thereof is continuously supplemented with from about 5% dextrose to about 20% dextrose (w/v), about 5% dextrose to about 15% dextrose (w/v), about 5% dextrose to about 10% dextrose (w/v), about 5% (w/v), about 10% dextrose (w/v).

In some embodiments, for rapid adjustment, a 50% (w/v) solution of dextrose can be administered as IV bolus.

In some embodiments, blood glucose is monitored during the administration of the dextrose solution. In some embodiments, blood glucose is monitored before the administration of the dextrose solution. In some embodiments, blood glucose is monitored after the administration of the dextrose solution. In some embodiments, the blood glucose is monitored every 60 min+/−20 min during the administration of the dextrose solution. In some embodiments, the blood glucose is monitored every 60 min+/−20 min during the first 24 hours of the dextrose supplementation. In some embodiments, the blood glucose is monitored every 120 min+/−20 min from 24 hours to 48 hours or more after the start of the dextrose supplementation. In some embodiments, the blood glucose is monitored according to the standard of care from 48 hours after the start of the dextrose supplementation. In some embodiments, the rate of dextrose supplementation is lowered or discontinued if a subject's blood glucose measures ≥150 mg/dL. In other embodiments, the rate of dextrose supplementation is lowered or discontinued if a subject's blood glucose measures ≥180 mg/dL.

In some embodiments, the Blood Glucose Management Plan (BGMP) is in accordance with current American Heart Association (AHA)/American Stroke Association (ASA) guidance for blood glucose management in acute ischemic stroke (Powers et al, Guidelines for the Early Management of Patients With Acute Ischemic Stroke: 2019 Update to the 2018 Guidelines for the Early Management of Acute Ischemic Stroke: A Guideline for Healthcare Professionals From the American Heart Association/American Stroke Association. Stroke. 2019).

In some embodiments, the subject in need thereof is monitored for blood glucose levels. In some embodiments, blood glucose (BG) monitoring is for a minimum of 48 hours under the following guidelines:

BG levels are utilized to determine the rate of dextrose supplementation based on Table below.

| | | BG Measurement | |
|---|---|---|---|
| BG Levels | Infusion Adjustment | Frequency Adjustment | Clinical Action |
| <60 mg/dL or | Increase D10 infusion | Recheck BG every | Administer |
| symptomatic | by 1 mL/kg/h | 15 minutes until | 1 amp of 50% |
| | | confirmed >80 mg/dL | dextrose |
| | | | injection |
| | | | intravenously |
| 60-69 mg/dL | Increase D10 infusion | Recheck BG every | May administer |
| | by 0.5 mL/kg/h | 15 minutes until | ½ amp of 50% |
| | | confirmed >80 mg/dL | dextrose |
| | | | injection |
| | | | intravenously at |
| | | | the discretion of |
| | | | the treating |
| | | | team |
| 70-79 mg/dL | Increase D10 infusion | Recheck BG every | |
| | by 0.2 mL/kg/h | 15 minutes until | |
| | | confirmed >80 mg/dL | |
| 80-150 mg/dL | Continue current D10 | Continue BGMP per | |
| | infusion rate | protocol | |
| >100 mg/dL for 4 | Continue current D10 | If later than 8 hours | |
| consecutive hours | infusion rate | postdose, glucose checks | |
| without increased | | may be modified to every | |
| dextrose administration | | 2 hours. | |
| >150 mg/dL on 2 | At investigator | Continue BGMP per | |
| consecutive | discretion, decrease | protocol | |
| readings separated | rate of or discontinue | | |
| by ≥30 minutes | D10 infusion and | | |
| | consider insulin drip | | |
| | or sliding scale per | | |
| | hospital protocol for | | |
| | hyperglycemia | | |

Abbreviations:
BG = blood glucose;
BGMP = Blood Glucose Management Plan;
D10 = 10% dextrose in water.

Following each administration of the IGF-1 or IGF-1 chimeric protein, the blood glucose level of the subject is checked at 30 minutes (±15 minutes), and hourly thereafter (±20 minutes) through 8 hours postdose. After 8 hours, if blood glucose levels are stable, glucose checks may be modified to every 2 hours (±20 minutes). Stable blood glucose levels are defined as blood glucose greater than 100 mg/dL for 4 consecutive hours without requiring increase to the rate of dextrose infusion.

Any change in clinical status suggesting a decrease in blood glucose levels (e.g., altered mental status post-dose) warrants an immediate glucose check, and if no longer stable, a return to hourly checks and dextrose rate adjustments until stabilized. If blood glucose levels were stabilized but are no longer stable:
If BG<80 mg/dL, recheck BG every 15 minutes and adjust/administer dextrose per Table 5 until BG confirmed >80 mg/dL.
When BG confirmed >80 mg/, resume hourly glucose checks if within 8 hours postdose.
If later than 8 hours postdose, resume hourly glucose checks again until BG is stabilized (BG>100 mg/dL for 4 consecutive hours without increased dextrose administration), then glucose checks may again be modified to every 2 hours.

In some embodiments, the BG level is measured by a central laboratory test, bedside test (e.g., i-STAT® system, arterial blood gas), or by fingerstick.

In some embodiments, before administration of IGF-1 or IGF-1 chimeric protein, an IV infusion of dextrose solution (e.g. D10) is started at a rate of 0.1 mL/kg/h.

In some embodiments, administration of the dextrose solution begins no more than 30 minutes before injection of the IGF-1 or IGF-1 chimeric protein.

In some embodiments, administration of the dextrose solution continues through at least Hour 48, unless BG levels are greater than 150 mg/dL on 2 consecutive readings separated by 30 minutes or more, then the dextrose administration is discontinued or the rate of administration of dextrose is decreased.

In some embodiments, if BG levels are decreasing or less than 80 mg/dL, the rate of administration of dextrose solution is increased.

In some embodiments, the dextrose solution can be 10% dextrose in water. In some embodiments, the dextrose solution can be switched to 5% dextrose in normal saline if there are clinical concerns for excess free water administration at the discretion of the site investigation team.

In some embodiments, if 50% dextrose injection is given for any reason, BG testing (fingerstick or laboratory glucose) should be conducted every 15 minutes until the BG level is >80 mg/dL.

### Additional Embodiments

The following additional embodiments are provided for illustrative purposes.

Embodiment 1: A method of treating a subject in need thereof with an IGF-1 chimeric protein, the method comprising:
administering a solution comprising an effective amount of dextrose prior to administration of an IGF-1 chimeric protein, wherein the administering is over a period of about 48 hours; and
administering a pharmaceutical composition comprising an effective amount of the IGF-1 chimeric protein,
wherein the effective amount of dextrose supports euglycemia.

Embodiment 2: The method of Embodiment 1, wherein the solution comprising the effective amount of dextrose is administered by infusion to the subject in need thereof.

Embodiment 3: The method of Embodiment 2, comprising administering the solution comprising the effective amount of dextrose at a starting infusion rate of about 0.1 mL/kg/h.

Embodiment 4: The method of Embodiment 3, further comprising adjusting the infusion rate at an incremental rate of +0.05 mL/kg/hr to +2 mL/kg/hr.

Embodiment 5: The method of any one of the preceding Embodiments, wherein the solution comprising the effective amount of dextrose comprises from about 5% to about 10% dextrose (w/v) in water or saline solution.

Embodiment 6: The method of any one of the preceding Embodiments, wherein the solution comprising the effective amount of dextrose is administered from about 1 min to about 30 min before the administration of the IGF-1 chimeric protein.

Embodiment 7: The method of any one of the preceding Embodiments, wherein the IGF-1 chimeric protein comprises a targeting domain comprising human annexin 5 (AnxV) or variant thereof, and an activator domain comprising insulin-like growth factor (IGF-1) or variant thereof.

Embodiment 8: The method of any one of the preceding Embodiments, wherein the IGF-1 chimeric protein further comprising a peptide, wherein the peptide extends the half-life of the IGF-1 chimeric protein.

Embodiment 9: The method of Embodiment 7 or 8, wherein the targeting domain is a non-internalizing variant of annexin 5.

Embodiment 10: The method of any one of the preceding Embodiments, wherein the IGF-1 chimeric protein is substantially not internalized by cells.

Embodiment 11: The method of any one of the preceding Embodiments, wherein the IGF-1 chimeric protein comprises a non-internalizing variant of annexin 5, wherein the non-internalizing variant of annexin 5 comprises one or more mutations, wherein the one or more mutations comprises a substitution at a position corresponding to C316 and optionally at one or more positions corresponding to R63, K70, K101, E138, D139, N160, and combinations thereof.

Embodiment 12: The method of any one of Embodiments 7-11, wherein the activator domain of IGF-1 chimeric protein is a variant of human insulin-like growth factor IGF-1 comprising one or more mutations, wherein the one or more mutations consist of a substitution at one or more positions corresponding to E3, Y24, Y31, Y60, and combinations thereof.

Embodiment 13: The method of any one of the preceding Embodiments, wherein the IGF-1 chimeric protein further comprises a half-life modulator comprising a variant of human serum albumin (HSA) comprising one or more mutations, wherein the one or more mutations consist of a substitution at one or more positions corresponding to C58 and N527, and combinations thereof.

Embodiment 14: The method of any one of the preceding Embodiments, wherein the IGF-1 chimeric protein comprises or consists of IGF1(E3R/Y31A)_lk7_HSA26-609(C58S/N527Q)_lk7_AnxV2-320(R63A/K70A/K101A/E138A/D139G/N160A/C316A).

Embodiment 15: The method of any one of the preceding Embodiments, wherein the IGF-1 chimeric protein comprises or consists of an amino acid sequence as set forth in SEQ ID NO: 24.

Embodiment 16: The method of any one of the preceding Embodiments, comprising administering descending effective amounts of the IGF-1 chimeric protein over a period of 2 days to 14 days or more.

Embodiment 17: The method of any one of the preceding Embodiments, wherein the administering of the effective amount of the IGF-1 chimeric protein inhibits apoptosis.

Embodiment 18: The method of any one of the preceding Embodiments, wherein the pharmaceutical composition further comprises at least one physiologically acceptable carrier.

Embodiment 19: The method of any one of the preceding Embodiments, wherein the subject in need thereof is a human.

Embodiment 20: The method of any one of the preceding Embodiments, wherein the subject in need thereof has acute CNS injury or acute cardiovascular injury (e.g., STEMI, Cardiac Arrest).

Embodiment 21: The method of any one of the preceding Embodiments, wherein the subject in need thereof has acute radiation sickness (e.g., GI-ARS, H-ARS).

Embodiment 22: The method of any one of the preceding Embodiments, wherein the subject in need thereof has chemical inhalation injury (e.g., sulfur mustards exposure).

Embodiment 23: The method of any one of the preceding Embodiments, wherein the subject in need thereof has dermatologic injuries (e.g., wound healing).

Embodiment 24: The method of any one of the preceding Embodiments, wherein the subject in need thereof has traumatic injuries (e.g., burn, crush, laceration, contusion, avulsion, concussion, fracture, amputation).

Embodiment 25: The method of any one of the preceding Embodiments, wherein the subject in need thereof has chronic neurodegenerative diseases (e.g., synucleinopathies, amyloidoses).

Embodiment 26: The method of any one of the preceding Embodiments, wherein the subject in need thereof is undergoing a procedure with risk of iatrogenic injury (e.g., transcatheter aortic valve replacement, percutaneous coronary intervention, coronary artery bypass grafting).

Embodiment 27: The method of any one of the preceding Embodiments, wherein the subject in need thereof is undergoing an organ transplantation procedure (e.g., skin, kidney, liver, heart, lung).

Embodiment 28: The method of any one of the preceding Embodiments, wherein the subject in need thereof is undergoing cosmetic dermatologic treatments (e.g., laser resurfacing).

Embodiment 29: An IGF-1 chimeric protein for use in a method of treating a subject in need thereof, the method comprising:
administering a solution comprising an effective amount of dextrose prior to administration of the IGF-1 chimeric protein, wherein the administering is over a period of about 48 hours; and administering a pharmaceutical composition comprising an effective amount of the IGF-1 chimeric protein,
   wherein the effective amount of dextrose supports euglycemia.

Embodiment 30: Dextrose for use in a method of treating a subject in need thereof with an IGF-1 chimeric protein, the method comprising:
administering a solution comprising an effective amount of said dextrose prior to administration of the IGF-1 chimeric protein, wherein the administering is over a period of about 48 hours; and administering a pharmaceutical composition comprising an effective amount of the IGF-1 chimeric protein,
   wherein the effective amount of dextrose supports euglycemia.

Embodiment 31: A combination of an IGF-1 chimeric protein and dextrose for use in a method of treating a subject in need thereof, the method comprising:
administering a solution comprising an effective amount of dextrose prior to administration of the IGF-1 chimeric protein, wherein the administering is over a period of about 48 hours; and administering a pharmaceutical composition comprising an effective amount of the IGF-1 chimeric protein,
   wherein the effective amount of dextrose supports euglycemia.

Embodiment 32: The IGF-1 chimeric protein, the dextrose or the combination of IGF-1 chimeric protein and dextrose of Embodiments 29-31, wherein the solution comprising the effective amount of dextrose is administered by infusion to the subject in need thereof.

Embodiment 33: The IGF-1 chimeric protein, the dextrose or the combination of IGF-1 chimeric protein and dextrose of Embodiment 32, comprising administering the solution comprising the effective amount of dextrose at a starting infusion rate of about 0.1 mL/kg/h.

Embodiment 34: The IGF-1 chimeric protein, the dextrose or the combination of IGF-1 chimeric protein and dextrose of Embodiment 33, further comprising adjusting the infusion rate at an incremental rate of +0.05 mL/kg/hr to +2 mL/kg/hr.

Embodiment 35: The IGF-1 chimeric protein, the dextrose or the combination of IGF-1 chimeric protein and dextrose of Embodiments 29-34, wherein the solution comprising the effective amount of dextrose comprises from about 5% to about 10% dextrose (w/v) in water or saline solution.

Embodiment 36: The IGF-1 chimeric protein, the dextrose or the combination of IGF-1 chimeric protein and dextrose of Embodiments 29-35, wherein the solution comprising the effective amount of dextrose is administered from about 1 min to about 30 min before the administration of the IGF-1 chimeric protein.

Embodiment 37: The IGF-1 chimeric protein, the dextrose or the combination of IGF-1 chimeric protein and dextrose of Embodiments 29-36, wherein the IGF-1 chimeric protein comprises a targeting domain comprising human annexin 5 (AnxV) or variant thereof, and an activator domain comprising insulin-like growth factor (IGF-1) or variant thereof.

Embodiment 38: The IGF-1 chimeric protein, the dextrose or the combination of IGF-1 chimeric protein and dextrose of Embodiments 29-37, wherein the IGF-1 chimeric protein further comprising a peptide, wherein the peptide extends the half-life of the IGF-1 chimeric protein.

Embodiment 39: The IGF-1 chimeric protein, the dextrose or the combination of IGF-1 chimeric protein and dextrose of Embodiments 37 or 38, wherein the targeting domain is a non-internalizing variant of annexin 5.

Embodiment 40: The IGF-1 chimeric protein, the dextrose or the combination of IGF-1 chimeric protein and dextrose of Embodiments 29-39, wherein the IGF-1 chimeric protein is substantially not internalized by cells.

Embodiment 41: The IGF-1 chimeric protein, the dextrose or the combination of IGF-1 chimeric protein and dextrose of Embodiments 29-40, wherein the IGF-1 chimeric protein comprises a non-internalizing variant of annexin 5, wherein the non-internalizing variant of annexin 5 comprises one or more mutations, wherein the one or more mutations comprises a substitution at a position corresponding to C316 and optionally at one or more positions corresponding to R63, K70, K101, E138, D139, N160, and combinations thereof.

Embodiment 42: The IGF-1 chimeric protein, the dextrose or the combination of IGF-1 chimeric protein and dextrose of Embodiments 37-41, wherein the activator domain of IGF-1 chimeric protein is a variant of human insulin-like growth factor IGF-1 comprising one or more mutations, wherein the one or more mutations consist of a substitution at one or more positions corresponding to E3, Y24, Y31, Y60, and combinations thereof.

Embodiment 43: The IGF-1 chimeric protein, the dextrose or the combination of IGF-1 chimeric protein and dextrose of Embodiments 29-42, wherein the IGF-1 chimeric protein further comprises a half-life modulator comprising a variant of human serum albumin (HSA) comprising one or more mutations, wherein the one or more mutations consist of a substitution at one or more positions corresponding to C58 and N527, and combinations thereof.

Embodiment 44: The IGF-1 chimeric protein, the dextrose or the combination of IGF-1 chimeric protein and dextrose of Embodiments 29-43, wherein the IGF-1 chimeric protein comprises or consists of IGF1(E3R/Y31A)_lk7_HSA26-609(C58S/N527Q)_lk7_AnxV2-320(R63A/K70A/K101A/E138A/D139G/N160A/C316A).

Embodiment 45: The IGF-1 chimeric protein, the dextrose or the combination of IGF-1 chimeric protein and dextrose of Embodiments 29-44, wherein the IGF-1 chimeric protein comprises or consists of an amino acid sequence as set forth in SEQ ID NO: 24.

Embodiment 46: The IGF-1 chimeric protein, the dextrose or the combination of IGF-1 chimeric protein and dextrose of Embodiments 29-45, comprising administering descending effective amounts of the IGF-1 chimeric protein over a period of 2 days to 14 days or more.

Embodiment 47: The IGF-1 chimeric protein, the dextrose or the combination of IGF-1 chimeric protein and dextrose of Embodiments 29-46, wherein the administering of the effective amount of the IGF-1 chimeric protein inhibits apoptosis.

Embodiment 48: The IGF-1 chimeric protein, the dextrose or the combination of IGF-1 chimeric protein and dextrose of Embodiments 29-47, wherein the pharmaceutical composition further comprises at least one physiologically acceptable carrier.

Embodiment 49: The IGF-1 chimeric protein, the dextrose or the combination of IGF-1 chimeric protein and dextrose of Embodiments 29-48, wherein the subject in need thereof is a human.

Embodiment 50: The IGF-1 chimeric protein, the dextrose or the combination of IGF-1 chimeric protein and dextrose of Embodiments 29-49, wherein the subject in need thereof has acute CNS injury or acute cardiovascular injury (e.g., STEMI, Cardiac Arrest).

Embodiment 51: The IGF-1 chimeric protein, the dextrose or the combination of IGF-1 chimeric protein and dextrose of Embodiments 29-50, wherein the subject in need thereof has acute radiation sickness (e.g., GI-ARS, H-ARS).

Embodiment 52: The IGF-1 chimeric protein, the dextrose or the combination of IGF-1 chimeric protein and dextrose of Embodiments 29-51, wherein the subject in need thereof has chemical inhalation injury (e.g., sulfur mustards exposure).

Embodiment 53: The IGF-1 chimeric protein, the dextrose or the combination of IGF-1 chimeric protein and dextrose of Embodiments 29-52, wherein the subject in need thereof has dermatologic injuries (e.g., wound healing).

Embodiment 54: The IGF-1 chimeric protein, the dextrose or the combination of IGF-1 chimeric protein and dextrose of Embodiments 29-53, wherein the subject in need thereof has traumatic injuries (e.g., burn, crush, laceration, contusion, avulsion, concussion, fracture, amputation).

Embodiment 55: The IGF-1 chimeric protein, the dextrose or the combination of IGF-1 chimeric protein and dextrose of Embodiments 29-54, wherein the subject in need thereof has chronic neurodegenerative diseases (e.g., synucleinopathies, amyloidoses).

Embodiment 56: The IGF-1 chimeric protein, the dextrose or the combination of IGF-1 chimeric protein and dextrose of Embodiments 29-55, wherein the subject in need thereof is undergoing a procedure with risk of iatrogenic injury (e.g., transcatheter aortic valve replacement, percutaneous coronary intervention, coronary artery bypass grafting).

Embodiment 57: The IGF-1 chimeric protein, the dextrose or the combination of IGF-1 chimeric protein and dextrose of Embodiments 29-56, wherein the subject in need thereof is undergoing an organ transplantation procedure (e.g., skin, kidney, liver, heart, lung).

Embodiment 58: The IGF-1 chimeric protein, the dextrose or the combination of IGF-1 chimeric protein and dextrose of Embodiments 29-57, wherein the subject in need thereof is undergoing cosmetic dermatologic treatments (e.g., laser resurfacing).

Embodiment 59: Use of an IGF-1 chimeric protein in the manufacture of a medicament for treating a subject in need thereof, wherein a solution comprising an effective amount of dextrose is administered prior to administration of a pharmaceutical composition comprising an effective amount of the IGF-1 chimeric protein IGF-1 chimeric protein, wherein administration of the solution is over a period of about 48, wherein the effective amount of dextrose supports euglycemia.

Embodiment 60: Use of dextrose with an IGF-1 chimeric protein in the manufacture of a medicament for treating a subject in need thereof, wherein a solution comprising an effective amount of the dextrose is administered prior to administration of a pharmaceutical composition comprising an effective amount of the IGF-1 chimeric protein IGF-1 chimeric protein, wherein administration of the solution comprising the effective amount of the dextrose is over a period of about 48, wherein the effective amount of dextrose supports euglycemia.

Embodiment 61: Use of a combination of IGF-1 chimeric protein and dextrose in the manufacture of a medicament for treating a subject in need thereof, wherein a solution comprising an effective amount of the dextrose is administered prior to administration of a pharmaceutical composition comprising an effective amount of the IGF-1 chimeric protein IGF-1 chimeric protein, wherein administration of the solution comprising the effective amount of the dextrose is over a period of about 48, wherein the effective amount of dextrose supports euglycemia.

Embodiment 63: The IGF-1 chimeric protein, the dextrose or the combination of IGF-1 chimeric protein and dextrose of Embodiments 59-61, wherein the solution comprising the effective amount of dextrose is administered by infusion to the subject in need thereof.

Embodiment 63: The IGF-1 chimeric protein, the dextrose or the combination of IGF-1 chimeric protein and dextrose of Embodiment 62, comprising administering the solution comprising the effective amount of dextrose at a starting infusion rate of about 0.1 mL/kg/h.

Embodiment 64: The IGF-1 chimeric protein, the dextrose or the combination of IGF-1 chimeric protein and dextrose of Embodiment 63, further comprising adjusting the infusion rate at an incremental rate of +0.05 mL/kg/hr to +2 mL/kg/hr.

Embodiment 65: The IGF-1 chimeric protein, the dextrose or the combination of IGF-1 chimeric protein and dextrose of Embodiments 59-64, wherein the solution comprising the effective amount of dextrose comprises from about 5% to about 10% dextrose (w/v) in water or saline solution.

Embodiment 66: The IGF-1 chimeric protein, the dextrose or the combination of IGF-1 chimeric protein and dextrose of Embodiments 59-65, wherein the solution comprising the effective amount of dextrose is administered from about 1 min to about 30 min before the administration of the IGF-1 chimeric protein.

Embodiment 67: The IGF-1 chimeric protein, the dextrose or the combination of IGF-1 chimeric protein and dextrose of Embodiments 59-66, wherein the IGF-1 chimeric protein comprises a targeting domain comprising human annexin 5 (AnxV) or variant thereof, and an activator domain comprising insulin-like growth factor (IGF-1) or variant thereof.

Embodiment 68: The IGF-1 chimeric protein, the dextrose or the combination of IGF-1 chimeric protein and dextrose of Embodiments 59-67, wherein the IGF-1 chimeric protein further comprising a peptide, wherein the peptide extends the half-life of the IGF-1 chimeric protein.

Embodiment 69: The IGF-1 chimeric protein, the dextrose or the combination of IGF-1 chimeric protein and dextrose of Embodiments 67 or 68, wherein the targeting domain is a non-internalizing variant of annexin 5.

Embodiment 70: The IGF-1 chimeric protein, the dextrose or the combination of IGF-1 chimeric protein and dextrose of Embodiments 59-69, wherein the IGF-1 chimeric protein is substantially not internalized by cells.

Embodiment 71: The IGF-1 chimeric protein, the dextrose or the combination of IGF-1 chimeric protein and dextrose of Embodiments 59-70, wherein the IGF-1 chimeric protein comprises a non-internalizing variant of annexin 5, wherein the non-internalizing variant of annexin 5 comprises one or more mutations, wherein the one or more mutations comprises a substitution at a position corresponding to C316 and optionally at one or more positions corresponding to R63, K70, K101, E138, D139, N160, and combinations thereof.

Embodiment 72: The IGF-1 chimeric protein, the dextrose or the combination of IGF-1 chimeric protein and dextrose of Embodiments 59-71, wherein the activator domain of IGF-1 chimeric protein is a variant of human insulin-like growth factor IGF-1 comprising one or more mutations, wherein the one or more mutations consist of a substitution at one or more positions corresponding to E3, Y24, Y31, Y60, and combinations thereof.

Embodiment 73: The IGF-1 chimeric protein, the dextrose or the combination of IGF-1 chimeric protein and dextrose of Embodiments 59-71, wherein the IGF-1 chimeric protein further comprises a half-life modulator comprising a variant of human serum albumin (HSA) comprising one or more mutations, wherein the one or more mutations consist of a substitution at one or more positions corresponding to C58 and N527, and combinations thereof.

Embodiment 74: The IGF-1 chimeric protein, the dextrose or the combination of IGF-1 chimeric protein and dextrose of Embodiments 59-74, wherein the IGF-1 chimeric protein comprises or consists of IGF1(E3R/Y31A)_lk7_HSA26-609(C58S/N527Q)_lk7_AnxV2-320(R63A/K70A/K101A/E138A/D139G/N160A/C316A).

Embodiment 75: The IGF-1 chimeric protein, the dextrose or the combination of IGF-1 chimeric protein and dextrose of Embodiments 59-74, wherein the IGF-1 chimeric protein comprises or consists of an amino acid sequence as set forth in SEQ ID NO: 24.

Embodiment 76: The IGF-1 chimeric protein, the dextrose or the combination of IGF-1 chimeric protein and dextrose of Embodiments 59-75, comprising administering descending effective amounts of the IGF-1 chimeric protein over a period of 2 days to 14 days or more.

Embodiment 77: The IGF-1 chimeric protein, the dextrose or the combination of IGF-1 chimeric protein and dextrose of Embodiments 59-76, wherein the administering of the effective amount of the IGF-1 chimeric protein inhibits apoptosis.

Embodiment 78: The IGF-1 chimeric protein, the dextrose or the combination of IGF-1 chimeric protein and dextrose of Embodiments 59-77, wherein the pharmaceutical composition further comprises at least one physiologically acceptable carrier.

Embodiment 79: The IGF-1 chimeric protein, the dextrose or the combination of IGF-1 chimeric protein and dextrose of Embodiments 59-78, wherein the subject in need thereof is a human.

Embodiment 80: The IGF-1 chimeric protein, the dextrose or the combination of IGF-1 chimeric protein and dextrose of Embodiments 59-79, wherein the subject in need thereof has acute CNS injury or acute cardiovascular injury (e.g., STEMI, Cardiac Arrest).

Embodiment 81: The IGF-1 chimeric protein, the dextrose or the combination of IGF-1 chimeric protein and dextrose of Embodiments 59-80, wherein the subject in need thereof has acute radiation sickness (e.g., GI-ARS, H-ARS).

Embodiment 82: The IGF-1 chimeric protein, the dextrose or the combination of IGF-1 chimeric protein and dextrose of Embodiments 59-81, wherein the subject in need thereof has chemical inhalation injury (e.g., sulfur mustards exposure).

Embodiment 83: The IGF-1 chimeric protein, the dextrose or the combination of IGF-1 chimeric protein and dextrose of Embodiments 59-82, wherein the subject in need thereof has dermatologic injuries (e.g., wound healing).

Embodiment 84: The IGF-1 chimeric protein, the dextrose or the combination of IGF-1 chimeric protein and dextrose of Embodiments 59-83, wherein the subject in need thereof has traumatic injuries (e.g., burn, crush, laceration, contusion, avulsion, concussion, fracture, amputation).

Embodiment 85: The IGF-1 chimeric protein, the dextrose or the combination of IGF-1 chimeric protein and dextrose of Embodiments 59-84, wherein the subject in need thereof has chronic neurodegenerative diseases (e.g., synucleinopathies, amyloidoses).

Embodiment 86: The IGF-1 chimeric protein, the dextrose or the combination of IGF-1 chimeric protein and dextrose of Embodiments 59-85, wherein the subject in need thereof is undergoing a procedure with risk of iatrogenic injury (e.g., transcatheter aortic valve replacement, percutaneous coronary intervention, coronary artery bypass grafting).

### EXAMPLES

The following Examples are offered by way of illustration and not by way of limitation. Unless otherwise specified, all reagents and solvents are of standard commercial grade and are used without further purification. Using routine modifications, the procedures provided in the following Examples may be varied by those of ordinary skill in the art to make and use other bi-specific fusion proteins and pharmaceutical compositions within the scope of the present disclosure.

### Example 1: Effect of Intravenous Scp776 (60 mg/Kg) on Blood Glucose in Healthy C57BL/6 Mice

The purpose of this study was to characterize the blood glucose profile following intended high dose of intravenous (i.v.) scp776 in the C57BL/6 mouse. These profiles serve as the baseline response to high dose scp776 administration and will be used for comparison as blood glucose supplementation strategies are developed. If deemed appropriate, blood glucose supplementation will be implemented in efficacy studies.

### Overview:

Scp776 (mg/mL in 20 mM Tris, 7.5% Sucrose, 0.02% polysorbate80, pH 7.5) was administered i.v. via tail vein injection in four C57BL/6 mice (age 64-65 days). For scp776 administration, mice were slightly anesthetized with isoflurane. Blood glucose was measured at baseline and subsequently monitored out to 8 hours post administration of scp776. At all time points, blood glucose measurements were made without anesthetization using tail bleed specimens and a handheld strip glucometer. During the procedure, mice had ad libitum access to food and water.

The procedure was then repeated with the same group of four C57BL/6 mice (age 86 days) 3 weeks later (t2).

TABLE 1
Summary of Animals

| | |
|---|---|
| Species: | C57BL/6 |
| Number: | 4 mice × 2 procedures |
| Age: | 75.2 ± 11.5 Days (15.3%) |
| Body Weight: | 25.6 ± 1.3 grams (5.0%) |
| Dose: | 60 mg/kg (12 mL/kg of 5 mg/mL |

### Results:

Scp776 administered i.v. at 60 mg/kg was well tolerated in all mice with no abnormal observations, despite the frequent handling. At this dose level, scp776 induced significant (but variable in magnitude) decreases in blood glucose level. In the second execution of the procedure, similar decreases in blood glucose were measured.

TABLE 2
Baseline Glucose

| | |
|---|---|
| Baseline Blood Glucose (n = 4) | 147.2 ± 13.2 mg/dL (9.0%) |
| Baseline Blood Glucose (n = 4) (t2) | 151.8 ± 17.9 mg/dL (11.8%) |
| Baseline Blood Glucose (combined): | 149.5 ± 14.8 mg/dL (9.9%) |
| (n = 8) | |

TABLE 3
Blood Glucose values

| Time Point | |
|---|---|
| (Dose at t = 0) | Blood Glucose μ ± σ (cv %) |
| Baseline | 149.5 ± 14.8 mg/dL (9.9%) |

| | | |
|---|---|---|
| 0.5 | hr | 91.5 ± 15.4 mg/dL (16.9%) |
| 1 | hr | 76.0 ± 19.5 mg/dL (25.7%) |
| 2 | hr | 69.8 ± 14.3 mg/dL (20.6%) |
| 3 | hr | 62.6 ± 15.3 mg/dL (24.4%) |
| 4 | hr | 68.1 ± 23.8 mg/dL (34.9%) |
| 5 | hr | 78.5 ± 18.0 mg/dL (22.9%) |
| 6 | hr | 85.8 ± 30.9 mg/dL (36.1%) |
| 7 | hr | 87.1 ± 38.7 mg/dL (44.4%) |
| 8 | hr | 100.6 ± 32.1 mg/dL (31.9%) |

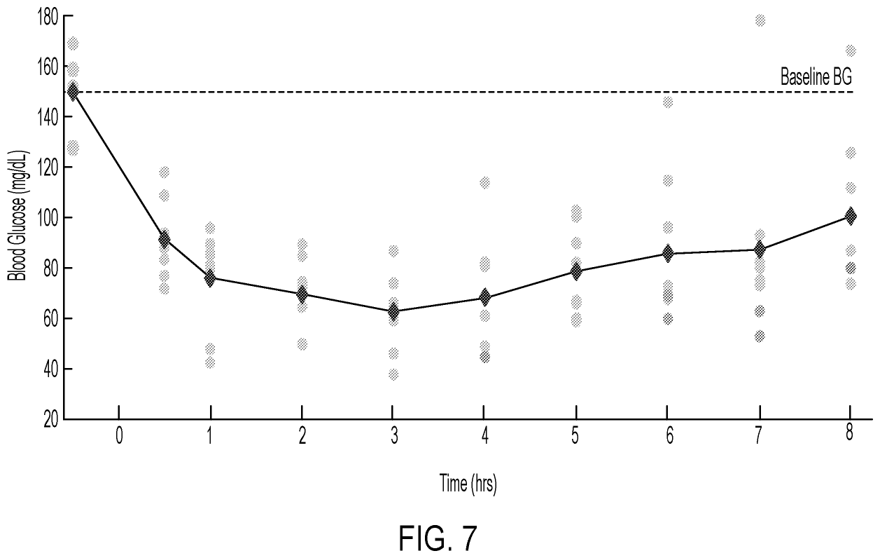

FIG. 7 shows the blood glucose trajectory. Scp776 administered i.v. at a dose of 60 mg/kg significantly decreases blood glucose in the C57BL/6 mouse. While the results are variable, the nadir of the averaged response occurs at three hours post scp776 administration. The duration of the response is surprising given the rate of clearance in rodents (t½<1.5 hrs). Contemplated glucose supplementation strategies would target maintaining a blood glucose level greater than 100 mg/dL through the first 8 hours.

### Example 2: Cerebroprotective Effect and Safety of Scp776, an IGF-1 Fusion Protein Targeted to Damaged Tissues, in Large Vessel Ischemic Stroke Patients Undergoing Thrombectomy without Thrombolysis

Scp776 is a targeted insulin-like growth factor 1 (IGF-1) fusion protein. In some embodiments, the targeted insulin-like growth factor 1 (IGF-1) fusion protein is used to inhibit apoptosis and limit cell death from hypoxia and reperfusion injury for acute ischemic stroke (AIS).

### Objective:

ARPEGGIO is evaluating the cerebroprotective potential of scp776 in subjects undergoing endovascular thrombectomy (EVT) for AIS.

### Design:

ARPEGGIO is a 2-part, Phase 2 randomized, double-blind, placebo-controlled trial of subjects with AIS, enrolling approximately 40 subjects in dose escalation (Part A) and 40 subjects for dose expansion (Part B).

### Population:

Key inclusion criteria are AIS within 16 hours, confirmed symptomatic intracranial occlusion in intracranial carotid artery and/or MI middle cerebral artery, and NIHSS≥6, while key exclusion criteria are acute intra-cerebral hemorrhage, poor/no collateral circulation, ASPECT score of 0-4, and current IV thrombolytic therapy.

In Part A, subjects are assigned 1:1:2 overall to Cohort 1 (1.9 mg/kg scp776), Cohort 2 (3.8 mg/kg scp776), or saline placebo. Doses of scp776 or saline placebo are IV administered once daily at 24 hour intervals. Subjects will receive glucose monitoring and dextrose supplementation for at least 48 hours. Safety data collected in Part A of ARPEGGIO will inform the dose selected for expansion in Part B.

In some embodiments, the IGF-1 activator domain in scp776 inhibits apoptosis and promotes cerebroprotection; the central human serum albumin scaffold extends the half-life of the chimeric protein and the Annexin V targeting domain binds phosphatidylserine on the surface of apoptotic cells.

In a non-human primate model of AIS, treatment with scp776 resulted in reduced lesion size, improved neurologic function, and a significant survival benefit. Scp776 was safe and well tolerated in two Phase 1 clinical studies.

Preclinical efficacy in non-human primate model of AIS:
Improved BBB Integrity during acute phase
Lesion size reduced by >30% at 72 hrs
Improved neurologic function
Mortality decreased five-fold in scp776 treatment group

In some embodiments, the chimeric proteins provided herein (e.g. scp776) do not bind to IGF binding proteins (IGFBPs) and are targeted to the injured cells at the site of the injury. In some embodiments, effectively targeting the injured cells result in limited side effects such as hypoglycemia, enlarged tonsils, and allergic reactions. In some embodiments, the chimeric proteins provided herein are efficiently delivered to mitigate damage as paracrine pro-survival factors. In some embodiments, the chimeric proteins provided herein (e.g. scp776) are engineered to drive-pro-survival signaling to injured cells.

In some embodiments, the chimeric proteins provided herein (e.g. scp776) are administered intravenously and have a circulating half-life of about 10 hours in healthy subjects.

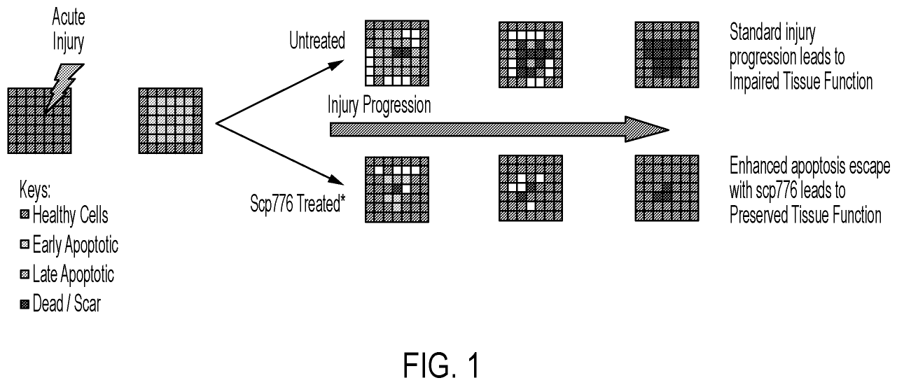

In some embodiments, targeted intervention during acute injury can change the destiny of injured cells and preserve tissue function. Following acute injury, cells at the injured core enter apoptosis, which expands to surrounding cells. Without intervention, the apoptotic core expands into the periphery, further impairing tissue function. Scp776 limits damage from an acute injury by promoting apoptosis escape in viable tissue, preventing the expansion of the injured core. See FIG. 1 depicting injury progression at the tissue level. Following the injury, the afflicted cell and surrounding areas enter the apoptotic pathway. In the absence of treatment, this spreads, culminating in a mass of dead cells that are paved over with scar tissue. The size of that scar determines the functional impact on the tissue. Scarring often results in irreparable impaired function. Scp776 is able to slow the spread of the injury, convert damaged cells to healthy, and ultimately reduce the size of the resulting scar-thereby preserving tissue function.

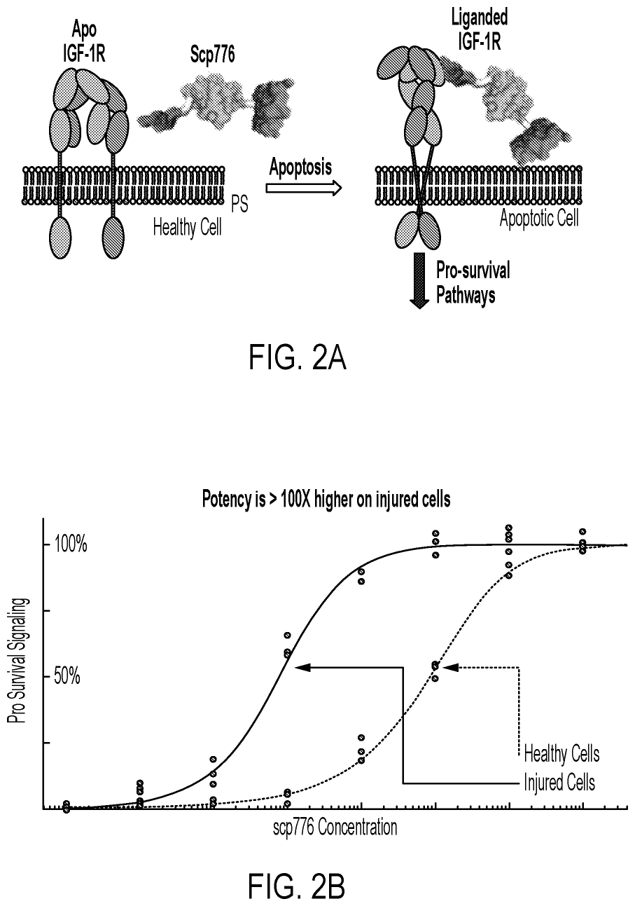

FIG. 2A-2B shows that scp776 inhibits the apoptotic pathway and promotes escape from apoptosis with greater potency than natural growth factors. Scp776 interacts strongly with phosphatidylserine (PS) shuffled to the cell exterior during apoptosis, activating pro-survival IGF-1R signaling at >100× lower concentrations in injured cells.

### Phase 1A

A single ascending dose, first in human Phase 1A trial, of scp776 was conducted in healthy volunteers to evaluate its safety, tolerability, and pharmacokinetic (PK) profile in humans. The study enrolled 24 healthy adult male subjects in 3 cohorts (6 active:2 placebo per cohort).

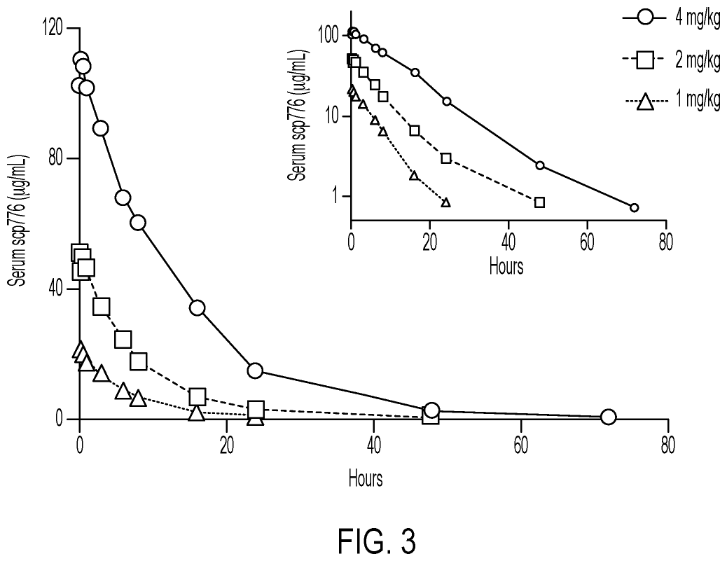

Serum concentration curves are shown for the three dose levels investigated in this study. In healthy human participants, the circulating half-life of scp776 is 10.6±1.4 hours. See FIG. 3. Results shows that scp776 is safe and well tolerated in healthy human subjects.

### Phase 1B

The Phase 1B study was a randomized, double-blind, placebo-controlled study of the safety and PK of single ascending and multiple doses of IV scp776 in healthy adults receiving a continuous infusion of dextrose. The study enrolled 44 healthy adult male and female subjects in 6 cohorts.

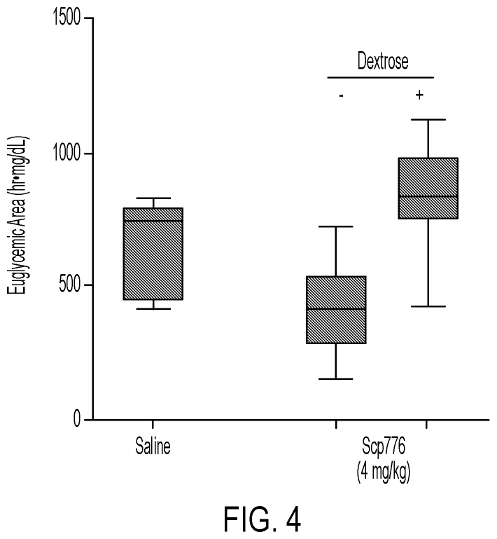

The supplemental dextrose infusion was implemented to limit the known blood glucose (BG) lowering effect of IGF-1. Comparison of the areas above euglycemia (70 mg/dL) in the Phase 1A and Phase 1B is shown. Supplemental dextrose in the Phase 1B significantly increased the euglycemic area, maintaining BG levels post administration of scp776. See FIG. 4. Bedside BG measurements were taken in the 24 hours post scp776 administration. Euglycemic area is calculated as the area under the BG curve above 70 mg/dL. The mean euglycemic area with dextrose supplementation is significantly greater (p=0.0041).

Additional results indicate that multidose regimens of scp776 were safe and well tolerated when administered with a continuous infusion of supplemental dextrose.

### Preclinical Efficacy of Scp776 is an NHP Model of AIS

Scp776 was studied in a double-blind non-human primate (Macaca fascicularis) transient middle cerebral artery occlusion model of AIS. Two clips were installed for four hours ischemia time creating a large infarct, small penumbra injury.

Scp776 (n=10) or placebo (n=12) was administered 30-minutes prior to reperfusion. All animals received continuously supplied supplemental dextrose via intravenous infusion pump. Dextrose in water (D10W) was infused at 0.5 mL/kg/hr. Infusion started 30-minutes before intravenous administration of the first dose of scp776 or placebo. The supplemental dextrose infusion continued at this rate for 120 hours following the first dose.

Endpoints included MRI imaging, neurologic function, and survival.

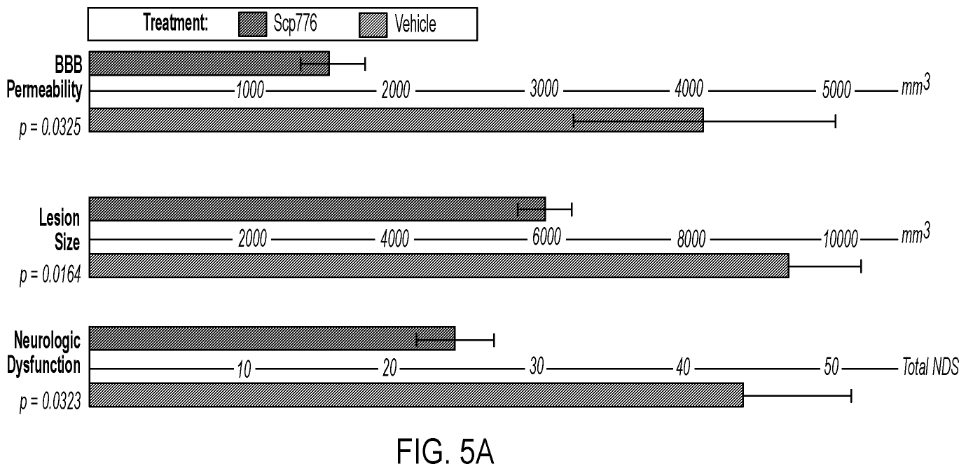

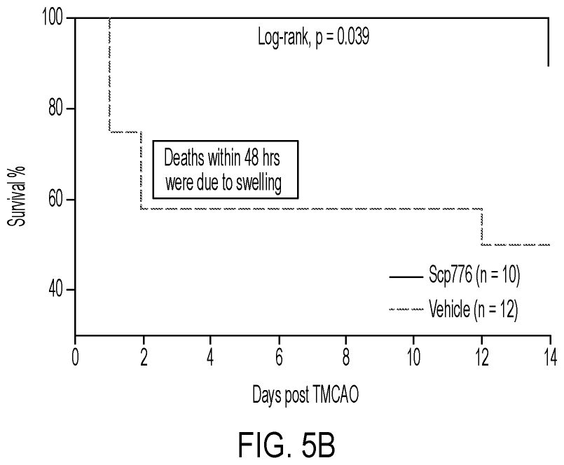

FIG. 5A and FIG. 5B show the preclinical efficacy of scp776 in an NHP model of AIS. FIG. 5A: Study PR0362-1 compared three groups: Placebo (n=12), Low Dose (24 mg/kg scp776 total; n=10), and High Dose (32 mg/kg scp776 total; n=10). Dose dependence was noted for all measured parameters. For simplicity, only the high dose is shown. Blood-brain barrier integrity, lesion size, and neurological deficit score are depicted for the high dose and placebo groups as group means+SEM. Multiple comparison of means (ANOVA with least significant difference) was performed with all three groups to determine significant effects. Values for monkeys that perished prior to a scheduled assessment were imputed to the maximum observed value. FIG. 5B: Survival curves for the high dose scp776 (n=10) and placebo (n=12) groups from Study PR0362-1 are plotted.

### Phase 2

Study design: ARPEGGIO is a Phase 2 randomized, placebo-controlled, double-blind study that will be conducted in 2 parts: sequential dose escalation in Part A, followed by dose expansion in Part B.

In Part A, approximately 40 evaluable subjects are assigned 1:1:2 overall to Cohort 1, Cohort 2, or placebo. Doses of scp776 are tested sequentially in 2 cohorts, each in parallel with a volume-matched placebo, randomized as 1:1 scp776: placebo within each cohort, to maintain the overall 1:1:2 ratio. Subjects receive 2 doses of either normal saline (placebo) or scp776 approximately 24 hours apart. In Part B, approximately 40 subjects are assigned 1:1 to the best therapeutic dose from Part A or placebo.

On Day 1, subjects who meet the eligibility criteria begin administration of study drug at a stroke center with an endovascular suite on-site. The qualifying brain imaging scan and Alberta stroke program early CT (ASPECT) score must be performed within 4 hours before the planned EVT. Study drug is intended to be administered within 1 hour of the baseline/qualifying scan, if possible, but no more than 4 hours after the qualifying scan. Study drug should be administered before the first pass with the EVT device, if possible, but before restoration of vessel flow. Preferably, at least 5 minutes should elapse after the end of the study drug administration before the opening of the vessel during the EVT procedure. The study drug is administered via slow IV injection over two minutes. The second dose of study drug is administered 24 hours after the first dose. Subjects remain hospitalized for at least 48 hours; data collected through Day 7 if still hospitalized. Subjects return for follow up visits on Days 30 and 90.

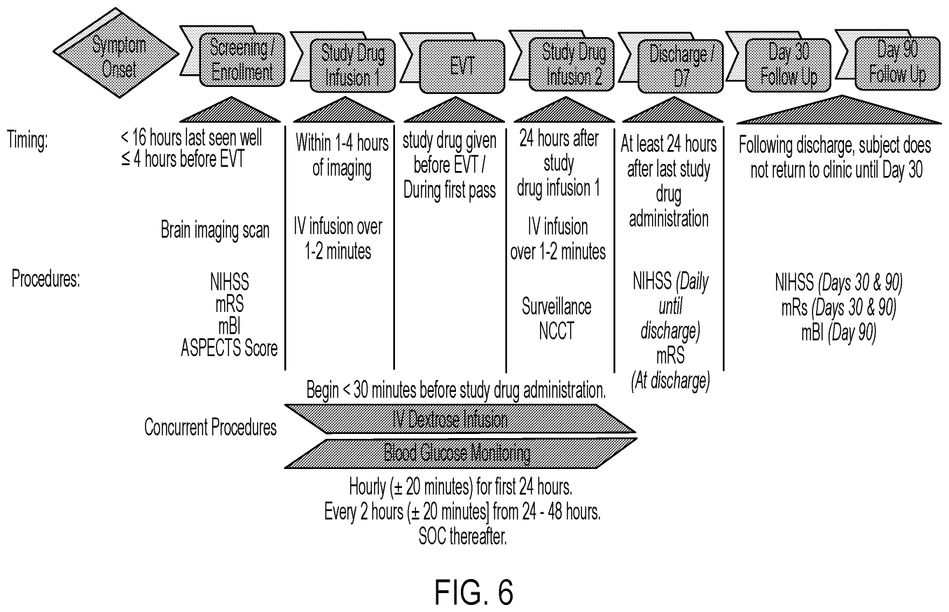

Because of the modified insulin-like growth factor signaling arm, scp776 can decrease blood glucose levels. Before administration of study drug (e.g. by 1 to 120 minutes), all subjects begin an IV infusion of 10% dextrose in water (D10) started at a rate of between 0.1 to 2.0 mL/kg/h. Dextrose administration can continue through at least 48 hours (for example, from about 48 hours to about 120 hours, from about 48 hours to about 72 hour, from 48 about hours to 96 about hours, from about 48 hours to about 120 hours, about 48 hours, about 72 hours, about 96 hours, about 120 hours) and managed per the protocol blood glucose management plan (BGMP). See FIG. 6.

### Inclusion Criteria:

Aged 18 years or older.
Acute ischemic stroke (AIS) intended for immediate endovascular treatment.
Disabling stroke defined as a baseline National Institutes of Health Stroke Score (NIHSS) greater than or equal to 6 at the time of randomization.
Confirmed symptomatic intracranial occlusion, based on qualifying imaging, at one or more of the following locations: Intracranial carotid artery and/or M1 middle cerebral artery (MCA). Onset AIS (last-seen-well) time to randomization time <16 hours.
Pre-AIS (24 hours prior to stroke onset) independent functional status in activities of daily living with modified Rankin Score of 0 or 1. Subject must be living in their own home, apartment, or seniors lodge where no nursing care is required.

### Exclusion Criteria:

Evidence of acute intra-cerebral hemorrhage on qualifying imaging, per radiology lab manual.
Poor/no collateral circulation (e.g. collateral score of 0 or 1).
ASPECT score of 0-4.
Current AIS is being treated with IV thrombolytic therapy or the subject has received thrombolytic therapy within the previous 24 hours.
Intent to use any endovascular thrombectomy device that is not FDA-approved.
Planned use of intra-arterial thrombolytic therapy.
Clinical history, past imaging or clinical judgment suggests that the intracranial occlusion is chronic or there is suspected intracranial dissection such that there is a predicted lack of success with endovascular intervention.

TABLE 4
Objectives and associated Endpoints

| Objectives | Associated Endpoints |
|---|---|
| Primary | |
| To measure the neuroprotective | Change in NIHSS from baseline to Day |
| capacity of scp776 | 7/discharge |
| Secondary | |
| To evaluate scp776 safety and | Frequency of TEAEs, SAEs, and drug- |
| tolerability | related AEs |
| To evaluate the effect on other | Proportion of subjects experiencing each of |
| assessments of efficacy | the following AESIs: |
| | Hypoglycemia |
| | Tachycardia |
| | Bleeding Events |
| | Change in NIHSS from baseline to Day 3 |
| | Proportion of subjects with Modified |
| | Rankin Scale score 02 at Day 7/discharge, |
| | and of 01 and 02 at Day 90 |
| | Change in Modified Rankin Scale score at |
| | Days 30 and 90 from baseline |
| | Day 30 survival |
| Pharmacokinetic (PK) | |
| To evaluate the pharmacokinetic | PK values and parameter estimates |
| profile of scp776 | calculated as by noncompartmental |
| | analysis as data permits |
| Exploratory | |
| To evaluate the exploratory | Change in NIHSS from baseline to Days 1 and 2 |
| efficacy and pharmacodynamics | Proportion of subjects with Barthel Index |
| of scp776 | between 90-100 on Day 90 |
| | Change in Barthel Index score from baseline to |
| | Day 90 |
| | Day 90 Survival |
| | Total dextrose infusion |
| | Change in lesion size from baseline to Day 4 |
| | Change in cerebral blood flow, cerebral blood |
| | volume, mean transit time, time to maximum |
| | perfusion, and time-to-peak flow, from baseline |
| | to Day 4 |

### Example 3: Effect of Starting Dextrose Infusion Rate on Total Dextrose Required to Support Euglycemia in Healthy Human Subjects Receiving Intravenous Infusions of Scp776

The purpose of this study was to identify dextrose supplementation parameters that promoted sustained euglycemia with minimal total dextrose infusion volumes.

Scp776 is a three-part fusion protein, which includes a modified IGF-1 signaling arm that is targeted to injured cells to enhance apoptosis escape and preserve healthy tissue. By targeting injured cells, scp776 can be administered at higher doses than IGF-1; however, glucose lowering effects of scp776 have been observed both in preclinical toxicity studies and in the clinical first-in-human single ascending dose study. A strategy of supplemental dextrose infusion and responsive glucose monitoring following scp776 dosing was developed in a multiple ascending dose study in healthy male and female participants. The blood glucose management strategy described herein can be deployed in acute ischemic stroke patients undergoing thrombectomy.

IGF-1 Based Therapies Can Decrease Blood Glucose: Insulin-like Growth Factor-1 (IGF-1) is structurally related to insulin and has widespread receptor binding throughout the body. IGF-1 is a hormone of clinical interest due to a broad range of important physiologic effects, including roles in neonatal growth and development, with primary postnatal targeting to skeletal muscle, as well as bone development, inhibition of apoptosis, and glucose regulation. IGF-1 has been evaluated for therapeutic lowering of glucose levels in diabetic patients; however, its therapeutic potential has been limited by the incidence of undesirable side effects in this population including jaw pain, arthralgias and tachycardia while its profound effects on glucose metabolism and potential for inducing hypoglycemia have limited its clinical development for other indications.

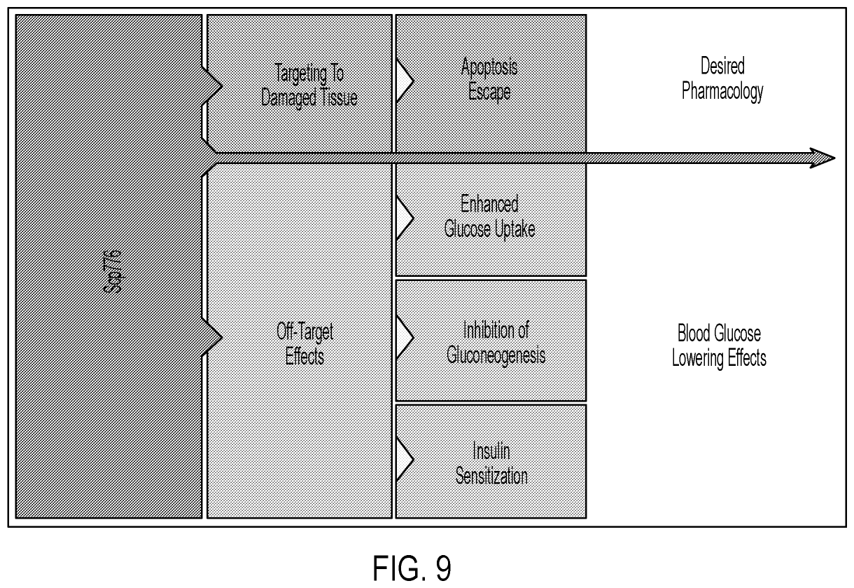

FIG. 9 shows that the inherent pharmacology and systemic effects of the IGF-1 component of scp776 can be addressed by dextrose supplementation.

Glycemic Management in Stroke: Hyperglycemia is common in the setting of acute ischemic stroke (AIS), due to both underlying diabetes and as a stress response to the stroke even in patients without diabetes. Conversely, in cases with prolonged time since last known well (LKW), stroke patients may be hypoglycemic due to prolonged fasting. Although hyperglycemia is a concern, studies which have used intensive glucose management via tight insulin control have not demonstrated improved functional outcomes or mortality, instead showing increased morbidity with hypoglycemic events. New therapies for the treatment of acute stroke must consider the comorbidity of dysglycemia in these patients and the potential confounding effects of these compounds, while following the AHA guidance on glucose management in AIS.

### Phase 1: Dextrose Infusion Strategy

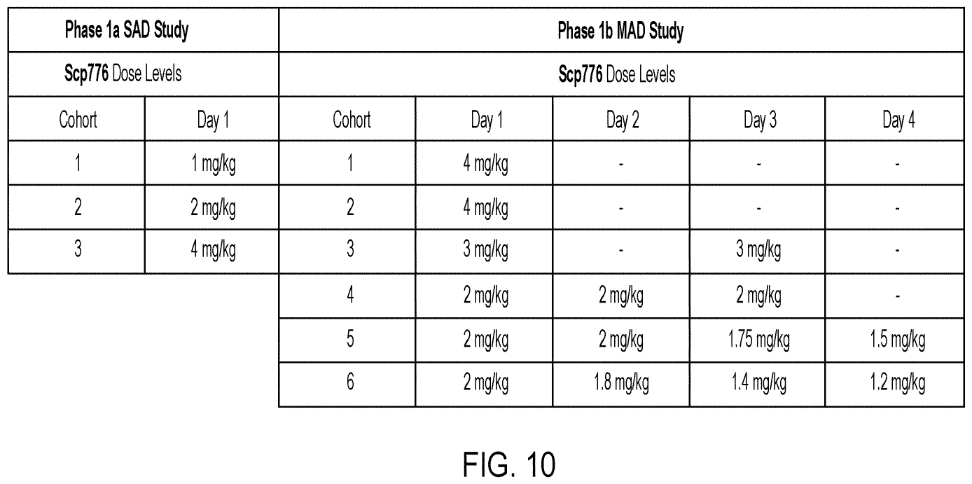

FIG. 10 shows Phase 1a single ascending dose (SAD) study and Phase 1b Multiple ascending dose (MAD) study. The Phase 1a SAD study enrolled only males. The Phase 1b MAD study enrolled males and females. Blood glucose levels were monitored at 30 minute intervals. Scheduled meals included: Breakfast (8 am), Lunch (12 μm), Dinner (5 μm), Snack (8 μm).

In the Phase 1a study, decreases in blood glucose were treated with oral glucose (e.g., juice).

In the Phase 1b study, IV dextrose was supplemented and changes in blood glucose were treated by modulation of the infusion rate.

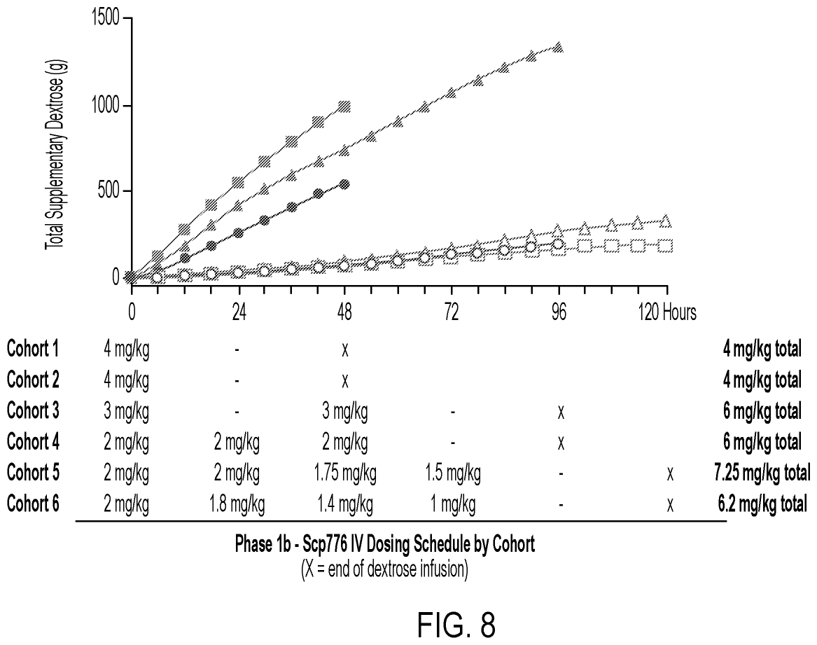

In the Phase 1B study SCP-CL-0002, healthy male and female subjects received supplemental IV dextrose prior to dosing. The blood glucose levels of individual subjects were responsive within standard monitoring intervals (i.e., 0.5 hour) to changes in the rates of dextrose infusion. While some subjects responded more rapidly than others, IV dextrose supplementation was able to successfully reverse episodes of hypoglycemia. During conduct, it was noted that high rates of infusion (i.e., 6 mL/kg/hr) could perpetuate the requirement for supplementation. It is noteworthy that the decrease in blood glucose appeared shortly after scp776 Tmax (~0.5 hour) and stabilized as serum scp776 concentrations decreased and dextrose supplementation was optimized. The tapering of doses combined with low initial dextrose infusion rates and incremental rate adjustments minimized total infusion volumes while providing glycemic control. These dose levels and dextrose supplementation strategies are feasible in a standard clinical setting wherein scp776 will be dosed in patients. See FIG. 8

In Cohorts 1-3, dextrose infusion rates were started high and rate adjustments were large. In Cohorts 4-6, the dextrose supplementation strategy was adjusted to initiate at low rates shortly before scp776 and make smaller, incremental rate adjustments in response to glucose monitoring. As evidenced, significantly less supplemental dextrose was required to maintain euglycemia in Cohorts 4-6.

In Cohorts 1, 2, and 3, subjects were administered total scp776 doses of 4 mg/kg, 4 mg/kg, and 6 mg/kg, respectively. These subjects started at 5% dextrose in normal saline (D5NS) or 10% dextrose (D10W) infusion rates of 1 mL/kg/h to 2 mL/kg/h up to two hours before administration of scp776. In these subjects, dextrose infusion rates were adjusted in large increments (i.e., from +1 mL/kg/hr to +2 mL/kg/hr) in response to measured blood glucose decreases of ≥5 mg/dL.

In Cohorts 4, 5, and 6, subjects were administered total scp776 doses of 6 mg/kg, 7.5 mg/kg, and 6.2 mg/kg, respectively. These subjects were started at D10W infusion rates within 30 minutes before administration of scp776. In these subjects, dextrose infusion rates were adjusted in small increments (i.e., from +0.1 mL/kg/hr to +0.5 mL/kg/hr) in response to measured blood glucose decreases of ≥5 mg/dL.

### Supplemental Dextrose Decreases Frequency of Hypoglycemic Events

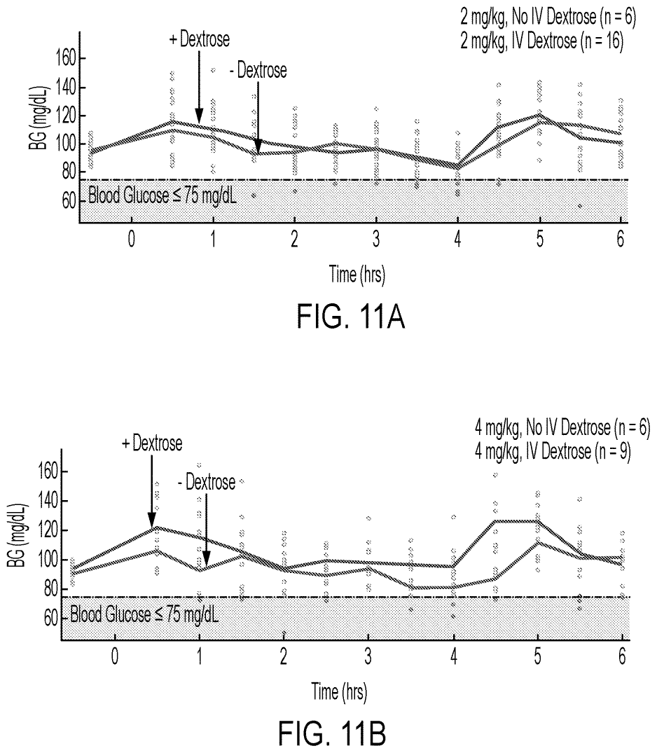

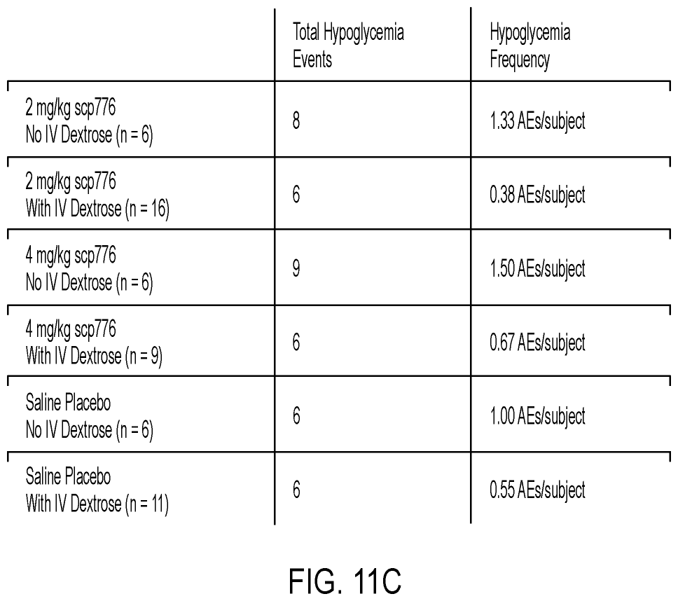

In the first-in-human study SCP-CL-0001, subjects received 2 or 4 mg/kg scp776 without constant dextrose supplementation. At matching dose levels in SCP-CL-0002 with IV dextrose supplementation, subjects experienced fewer hypoglycemia events and better maintained normal blood glucose levels in the 6-hour period following dosing. For reference, hypoglycemia events in the Saline Placebo group are also presented. (Note: Meals were scheduled at t=0 & 4 hours). See FIGS. 11A-11C

For all subjects that received scp776 with supplemental IV dextrose, no grade 2 or higher hypoglycemic events (CTCAE criteria of ≤54 mg/dL) were observed in the period following dosing, indicating that euglycemia was maintained with minimal excursions to grade 1 hypoglycemia (CTCAE criteria of ≤75 mg/dL).

FIG. 8 presents the average total volume of supplemental dextrose solutions infused for subjects in each of the Cohorts1-6.

Cohort 1 was administered a single dose of scp776 at 4 mg/kg. Cohort 2 was administered a single dose of scp776 at 4 mg/kg. Cohort 3 was administered two doses of scp776 at 3 mg/kg on day 1 and day 3. Cohort 4 was administered three doses of scp776 at 2 mg/kg on days 1, 2 and 3. Cohort 5 was administered two doses of scp776 at 2 mg/kg on days 1 and 2, a dose of scp776 at 1.75 mg/kg on day 3 and a dose of scp776 at 1.5 mg/kg on day 4. Cohort 6 was administered a dose of scp776 at 2 mg/kg on day 1, a dose of scp776 at 1.8 mg/kg on day 2, a dose of scp776 at 1.4 mg/kg on day 3, a dose of scp776 at 1 mg/kg on day 4. Subjects in Cohorts 1, 2, or 3 received, on average, approximately 7,000 mL of dextrose supplementing solutions in the first 48 hours. Subjects in Cohorts 4, 5, or 6 received, on average, approximately 1,000 mL of dextrose supplementing solutions in the first 48 hours.

In support of the three dose regimen in Cohort 4, subjects, on average, received approximately 1,600 mL of dextrose supplementing solutions in the 96 hours following the first scp776 administration.

In support of the four dose regimens in Cohorts 5 and 6, subjects, on average, received approximately 2,000 mL of dextrose supplementing solutions in the 120 hours following the first scp776 administration.

The marked difference in the total volumes of dextrose supplementing solutions necessary to support euglycemia in Cohorts 1-3 compared to Cohorts 4-6 is attributed to modifications to the blood glucose management program.

Because hypoglycemia is a known side effect of scp776, all subjects in this trial received supplemental dextrose via controlled IV infusion. The supplemental dextrose infusion was implemented to minimize possible risks associated with sustained decreases in blood glucose level. The supplemental dextrose solutions and the route of administration were selected to reproduce the clinical setting of eventual diseased patients who will receive scp776.

An adaptive approach for the supplemental dextrose infusion was used during the course of the study. In general, the subjects did better when the dextrose infusion was started close to the first dose as opposed to 30 minutes, or more, prior to dosing. Initial dextrose infusion parameters (e.g., starting rate, dextrose solution) were consistent within a given dose group of subjects. As the study progressed, initial parameters were modified based on the blood glucose control in previous cohorts and the adjustments to the scp776 dosing regimen. The initial parameters for dextrose infusion are summarized below by cohort and/or dose group.

In Cohort 1, subjects were to receive a continuous infusion of 5% dextrose in normal saline (DNS) at a rate of 0.5 mL/kg/hr immediately prior to dosing of scp776 or placebo and continuing for the first 48 hours after dosing. However, due to steep downward trends in blood glucose levels requiring increased amounts of dextrose, it was determined that the large volume of D5NS required to mitigate the low blood glucose levels may not be appropriate or manageable by all subjects. For subjects in whom D5NS was inappropriate, a similar dextrose supplementation (e.g., 10% dextrose in sterile water solution (D10W)) strategy was implemented. In light of this, when the Cohort 1 single dose level of 4 mg/kg was repeated in Cohort 2, the initial supplemental dextrose infusion was modified to D10W at a starting rate of 2 mL/kg/hr.

In Cohorts 2 through 4, subjects received a continuous infusion of D10W at an initial rate of less than or equal to 2 mL/kg/hr prior to dosing of scp776 or placebo and continuing for the first 48 hours after the last dose (i.e., 96 hours after first dosing for Cohorts 3 and 4). Additionally, in the first group of subjects dosed in Cohort 2, the dextrose infusions were started approximately 1 hour prior to dosing of scp776 or placebo; however, this appeared to result in a priming effect on endogenous insulin. Thus, in the subsequent dosing group and cohorts the start of the dextrose infusion was reverted back to be immediately prior to dosing of scp776 or placebo. Although the downward trending of blood glucose levels decreased compared to the initial steep downward trends in Cohort 1 (i.e., trending improved), large adjustments in the D10W infusion rate were still required to mitigate low blood glucose levels for most subjects in Cohort 2 (single dose of 4 mg/kg scp776 or placebo) and for the first 4 subjects in Cohort 3 (3 mg/kg scp776 or placebo QOD for 2 doses). For the remaining subjects dosed in Cohort 3, the initial supplemental dextrose (D10W) infusion rate was decreased to 0.5 mL/kg/hr and smaller adjustments in the D10W infusion rates were used in response to decreases in blood glucose levels. This resulted in improved blood glucose control with less supplemental dextrose administrated.

By the start of Cohort 4, the initial supplemental D10W infusion rate was low requiring smaller/finer adjustments in the D10W infusion rate to mitigate low blood glucose levels. In Cohorts 5 and 6, subjects received a continuous infusion of D10W at an initial rate of 0.1 mL/kg/hr immediately prior to dosing of scp776 or placebo. The dextrose infusion can then be adjusted, paused, or discontinued at any time following administration of the first dose for subjects in Cohorts 5 and 6 per protocol Blood Glucose Management Plan (BGMP, Table 5 and Example 4) with minor incremental changes in infusion rate based on bedside glucose measurements.

TABLE 5
Dextrose infusion strategy guidelines for management of euglycemia.

| | | BG Measurement | |
|---|---|---|---|
| BG Levels | Infusion Adjustment | Frequency Adjustment | Clinical Action |
| <60 mg/dL or | Increase D10 infusion | Recheck BG every | Administer |
| symptomatic | by 1 mL/kg/h | 15 minutes until | 1 amp of 50% |
| | | confirmed >80 mg/dL | dextrose |
| | | | injection |
| | | | intravenously |
| 60-69 mg/dL | Increase D10 infusion | Recheck BG every | May administer |
| | by 0.5 mL/kg/h | 15 minutes until | ½ amp of 50% |
| | | confirmed >80 mg/dL | dextrose |
| | | | injection |
| | | | intravenously at |
| | | | the discretion of |
| | | | the treating |
| | | | team |
| 70-79 mg/dL | Increase D10 infusion | Recheck BG every | |
| | by 0.2 mL/kg/h | 15 minutes until | |
| | | confirmed >80 mg/dL | |
| 80-150 mg/dL | Continue current D10 | Continue BGMP per | |
| | infusion rate | protocol | |
| >100 mg/dL for 4 | Continue current D10 | If later than 8 hours | |
| consecutive hours | infusion rate | postdose, glucose checks | |
| without increased | | may be modified to every | |
| dextrose administration | | 2 hours. | |
| >150 mg/dL on 2 | At investigator | Continue BGMP per | |
| consecutive | discretion, decrease | protocol | |
| readings separated | rate of or discontinue | | |
| by ≥30 minutes | D10 infusion and | | |
| | consider insulin drip | | |
| | or sliding scale per | | |
| | hospital protocol for | | |
| | hyperglycemia | | |

Abbreviations:
BG = blood glucose;
BGMP = Blood Glucose Management Plan;
D10 = 10% dextrose in water.

Based on our experience in the first in human study of scp776 (SCP-CL-0001), a stringent Blood Glucose Management Plan (BGMP) was implemented in the Phase 1b MAD study (SCP-CL-0002). Continuous glucose monitoring devices and bedside point-of-care measurements were used to monitor BG levels in all subjects, they received supplemental IV dextrose, with rates of infusion adjusted to maintain euglycemia.

The experiences in both Phase 1 studies have informed the design of the BGMP that is currently deployed in the Phase 2 study of scp776 in acute ischemic stroke patients.
In the stroke setting, there will be variability in the timing of most recent meals and baseline blood glucose levels.
Blood glucose decreases are most likely to occur within the first 6 hours following scp776 administration and in subjects with lower baseline levels.
Supplemental dextrose infusion should be started at a low rate shortly before administration of scp776.
Responsive and incremental adjustments to the dextrose infusion rate are more effective than delayed or large rate adjustments.
Oral feeding of well-balanced meals should be encouraged.

### Example 4: Blood Glucose Management Plan

A subset of subjects who receive the study drug scp776 may develop hypoglycemia, which will be managed with a combination of frequent glucose checks and supplemental dextrose.

At all times, clinical judgment should be used for glucose management, and the BGMP should not interfere with institutional standards of clinical care.

In some embodiments, protocol-specified blood glucose management continues through 48 hours after the first study drug administration, after which patients may continue dextrose supplementation and blood glucose monitoring per standard of care.

### Glucose Monitoring and Management

All subjects administered study drug have blood glucose (BG) monitoring for a minimum of 48 hours under the following guidelines:

BG levels are utilized to determine the rate of dextrose supplementation based on Table 5.

Following each administration of study drug, all subjects have a blood glucose check at 30 minutes (±15 minutes), and hourly thereafter (±20 minutes) through 8 hours postdose. After 8 hours, if blood glucose levels are stable, glucose checks may be modified to every 2 hours (±20 minutes). Stable blood glucose levels are defined as blood glucose >100 mg/dL for 4 consecutive hours without requiring increases to the rate of dextrose infusion.

Any change in clinical status suggesting a decrease in blood glucose (e.g., altered mental status post-dose) warrants an immediate glucose check, and if no longer stable, a return to hourly checks and dextrose rate adjustments until stabilized.

If blood glucose levels were stabilized but are no longer stable:
If BG<80 mg/dL, recheck BG every 15 minutes and adjust/administer dextrose per Table 5 until BG confirmed >80 mg/dL.
When BG confirmed >80 mg/, resume hourly glucose checks if within 8 hours postdose.
If later than 8 hours postdose, resume hourly glucose checks again until BG is stabilized (BG>100 mg/dL for 4 consecutive hours without increased dextrose administration), then glucose checks may again be modified to every 2 hours.

BG can be measured by a central laboratory test, bedside test (e.g., i-STAT® system, arterial blood gas), or by fingerstick.

### Dextrose Infusion

Before administration of study drug, all subjects begin an IV infusion of D10 started at a rate of 0.1 mL/kg/h.

The dextrose infusion should begin no more than 30 minutes before study drug injection.

Dextrose administration continues through at least Hour 48, unless BG levels are >150 mg/dL on 2 consecutive readings separated by ≥30 minutes, then the D10 can be discontinued or the rate can be decreased.

If BG levels are decreasing or <80 mg/dL, the D10 rate is increased.

D10 may be switched to 5% dextrose in normal saline if there are clinical concerns for excess free water administration at the discretion of the site investigation team. All changes in dextrose infusion rate should be noted in the case report form.

The clinical team may deviate from the protocol in Table 5 if the rate of decrease of BG levels is considered to be rapid, and should note the reason for the change from the plan.

If 50% dextrose injection is given for any reason, BG testing (fingerstick or laboratory glucose) should be conducted every 15 minutes until the BG level is >80 mg/dL.

If the rate of D10 infusion exceeds 1 mL/kg/h, the medical monitor for the study should be contacted.

Specific examples of compositions, methods and kits have been described herein for purposes of illustration. These are only examples. The technology provided herein can be applied to systems other than the example systems described above. Many alterations, modifications, additions, omissions, and permutations are possible within the practice of this invention. This disclosure includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

### INCORPORATION BY REFERENCE

All publications, patents and sequence database entries mentioned herein are hereby incorporated by reference in their entirety as if each individual publication or patent was specifically and individually indicated to be incorporated by reference.

1. A method of treating a subject in need thereof with an IGF-1 chimeric protein, the method comprising:
administering a solution comprising an effective amount of dextrose prior to administration of an IGF-1 chimeric protein, wherein the administering is over a period of about 48 hours; and
administering a pharmaceutical composition comprising an effective amount of the IGF-1 chimeric protein,
wherein the effective amount of dextrose supports euglycemia.

2. The method of claim 1, wherein the solution comprising the effective amount of dextrose is administered by infusion to the subject in need thereof.

3. The method of claim 2, comprising administering the solution comprising the effective amount of dextrose at a starting infusion rate of about 0.1 mL/kg/h.

4. The method of claim 3, further comprising adjusting the infusion rate at an incremental rate of +0.05 mL/kg/hr to +2 mL/kg/hr.

5. The method of claim 1 or claim 2, wherein the solution comprising the effective amount of dextrose comprises from about 5% to about 10% dextrose (w/v) in water or saline solution.

6. The method of claim 1 or claim 2, wherein the solution comprising the effective amount of dextrose is administered from about 1 min to about 30 min before the administration of the IGF-1 chimeric protein.

7. The method of claim 1 or claim 2, wherein the IGF-1 chimeric protein comprises a targeting domain comprising human annexin 5 (AnxV) or variant thereof, and an activator domain comprising insulin-like growth factor (IGF-1) or variant thereof.

8. The method of claim 7, wherein the IGF-1 chimeric protein further comprising a peptide, wherein the peptide extends the half-life of the IGF-1 chimeric protein.

9. The method of claim 7, wherein the targeting domain is a non-internalizing variant of annexin 5.

10. The method of claim 7, wherein the IGF-1 chimeric protein is substantially not internalized by cells.

11. The method of claim 7, wherein the IGF-1 chimeric protein comprises a non-internalizing variant of annexin 5, wherein the non-internalizing variant of annexin 5 comprises one or more mutations, wherein the one or more mutations comprises a substitution at a position corresponding to C316 and optionally at one or more positions corresponding to R63, K70, K101, E138, D139, N160, and combinations thereof.

12. The method of claim 7, wherein the activator domain of IGF-1 chimeric protein is a variant of human insulin-like growth factor IGF-1 comprising one or more mutations, wherein the one or more mutations consist of a substitution at one or more positions corresponding to E3, Y24, Y31, Y60, and combinations thereof.

13. The method of claim 7, wherein the IGF-1 chimeric protein further comprises a half-life modulator comprising a variant of human serum albumin (HSA) comprising one or more mutations, wherein the one or more mutations consist of a substitution at one or more positions corresponding to C58 and N527, and combinations thereof.

14. The method of claim 7, wherein the IGF-1 chimeric protein comprises or consists of IGF1(E3R/Y31A)_lk7_HSA26-609(C58S/N527Q)_lk7_AnxV2-320(R63A/K70A/K101A/E138A/D139G/N160A/C316A).

15. The method of claim 7, wherein the IGF-1 chimeric protein comprises or consists of an amino acid sequence as set forth in SEQ ID NO: 24.

16. The method of claim 1 or claim 2, comprising administering descending effective amounts of the IGF-1 chimeric protein over a period of 2 days to 14 days or more.

17. The method of claim 1 or claim 2, wherein the administering of the effective amount of the IGF-1 chimeric protein inhibits apoptosis.

18. The method of claim 1 or claim 2, wherein the pharmaceutical composition further comprises at least one physiologically acceptable carrier.

19. The method of claim 1 or claim 2, wherein the subject in need thereof is a human.

20. The method of claim 19, wherein the subject in need thereof has acute CNS injury.

21. The method of claim 19, wherein the subject in need thereof has acute cardiovascular injury.

22. The method of claim 19, wherein the subject in need thereof has acute radiation sickness.

23. The method of claim 19, wherein the subject in need thereof has chemical inhalation injury.

24. The method of claim 19, wherein the subject in need thereof has dermatologic injuries.

25. The method of claim 19, wherein the subject in need thereof has traumatic injuries.

26. The method of claim 19, wherein the subject in need thereof has chronic neurodegenerative diseases.

27. The method of claim 19, wherein the subject in need thereof is undergoing a procedure with risk of iatrogenic injury.

28. The method of claim 19, wherein the subject in need thereof is undergoing an organ transplantation procedure.

29. The method of claim 19, wherein the subject in need thereof is undergoing cosmetic dermatologic treatments.

30. An IGF-1 chimeric protein for use in a method of treating a subject in need thereof, the method comprising:
administering a solution comprising an effective amount of dextrose prior to administration of the IGF-1 chimeric protein, wherein the administering is over a period of about 48 hours; and administering a pharmaceutical composition comprising an effective amount of the IGF-1 chimeric protein,
wherein the effective amount of dextrose supports euglycemia.

31. Dextrose for use in a method of treating a subject in need thereof with an IGF-1 chimeric protein, the method comprising:
administering a solution comprising an effective amount of said dextrose prior to administration of the IGF-1 chimeric protein, wherein the administering is over a period of about 48 hours; and administering a pharmaceutical composition comprising an effective amount of the IGF-1 chimeric protein,
wherein the effective amount of dextrose supports euglycemia.

32. A combination of an IGF-1 chimeric protein and dextrose for use in a method of treating a subject in need thereof, the method comprising:
administering a solution comprising an effective amount of dextrose prior to administration of the IGF-1 chimeric protein, wherein the administering is over a period of about 48 hours; and administering a pharmaceutical composition comprising an effective amount of the IGF-1 chimeric protein,
wherein the effective amount of dextrose supports euglycemia.

33. Use of an IGF-1 chimeric protein in the manufacture of a medicament for treating a subject in need thereof, wherein a solution comprising an effective amount of dextrose is administered prior to administration of a pharmaceutical composition comprising an effective amount of the IGF-1 chimeric protein IGF-1 chimeric protein, wherein administration of the solution is over a period of about 48, wherein the effective amount of dextrose supports euglycemia.

34. Use of dextrose with an IGF-1 chimeric protein in the manufacture of a medicament for treating a subject in need thereof, wherein a solution comprising an effective amount of the dextrose is administered prior to administration of a pharmaceutical composition comprising an effective amount of the IGF-1 chimeric protein IGF-1 chimeric protein, wherein administration of the solution comprising the effective amount of the dextrose is over a period of about 48, wherein the effective amount of dextrose supports euglycemia.

35. Use of a combination of IGF-1 chimeric protein and dextrose in the manufacture of a medicament for treating a subject in need thereof, wherein a solution comprising an effective amount of the dextrose is administered prior to administration of a pharmaceutical composition comprising an effective amount of the IGF-1 chimeric protein IGF-1 chimeric protein, wherein administration of the solution comprising the effective amount of the dextrose is over a period of about 48, wherein the effective amount of dextrose supports euglycemia.
